(19) United States
(12) Patent Application Publication
(10) Pub. No.: US 2004/0132679 A1
(43) Pub. Date: Jul. 8, 2004

(54) Induction of pancreatic islet formation

(75) Inventors: Lawrence Chan, Houston, TX (US); Hideto Kojima, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX (US)

(21) Appl. No.: 10/654,102

(22) Filed: Sep. 3, 2003

Related U.S. Application Data
(60) Provisional application No. 60/407,743, filed on Sep. 3, 2002.

(51) Int. Cl. A61K 48/00; A61K 38/18

(52) U.S. Cl. 514/44; 514/12

(57) ABSTRACT

The present invention is directed to compositions of an islet cell differentiation transcription factor polypeptide, or any of its homologs or orthologs, as a therapeutic agent for the treatment of diabetes, more specifically insulin-dependent diabetes. The methods and compositions of the present invention provide an increase in glucose tolerance, an increase in insulin, and/or an increase in insulin-producing cells in the host.

The present application claims priority to U.S. Provisional Patent Application Serial No. 60/407,743, filed Sep. 3, 2002, which is incorporated by reference herein in its entirety.

### STATEMENT REGARDING FEDERALLY SPONSORED RESEARCH OR DEVELOPMENT

The work herein was supported by grants from the United States Government, National Institute of Health, grant numbers HL 51586 and HL 16512. Therefore, the Government may have certain rights in the invention.

### FIELD OF THE INVENTION

The present invention is directed to the fields of cellular and molecular biology, gene therapy and medicine, and is directed to compositions of an islet cell differentiation transcription factor polypeptide, or any of its homologs or orthologs, and/or a nucleic acid encoding therefor, as a therapeutic agent for the treatment of diabetes, more specifically insulin-dependent diabetes.

### BACKGROUND OF THE INVENTION

Diabetes mellitus type 1, or insulin-dependent diabetes, results from a genetically conferred vulnerability that causes a primary deficiency of insulin in the body. This deficiency of insulin is believed to be the consequence of destruction of a specialized population of cells that produce insulin in the body, pancreatic beta-cells. An autoimmune process may also contribute to beta-cell damage. The resulting lack of insulin and excess of glucagon augments glucose production, and the efficiency of peripheral glucose use is reduced until a new equilibrium between these processes is reached at a very high plasma glucose level. Because of the high plasma glucose levels, the filtered load of glucose exceeds the renal tubular capacity for reabsorption. Thus, glucose is excreted in the urine in large quantities. This osmotic effect causes increased excretion of water and salts and frequent urination. The goal of insulin treatment is to systemically lower plasma levels of glucose, free fatty acids, and ketoacids to normal levels and to reduce urine nitrogen losses. Conventional methods of achieving a homeostatic level of insulin in a diabetic (type 1) patient target direct actions targeting increasing insulin and, also, diminishing the secretion of glucagon.

Recently, efforts to investigate diabetes at the molecular level have increased. For example, genetic screening for persons at risk for type I diabetes has been described in U.S. Pat. No. 6,326,141 to Kahn et al., which correlated the increased expression of a muscle glycogen phosphorylase gene and a human elongation factor 1-alpha gene for increased risk for developing type I diabetes.

The recent confirmation that alpha and beta-cells are derived from an islet progenitor cell and follow independent lineage pathways rather than arising from a common mutihormonal progenitor cell has been used in strategies to provide a replenishable supply of insulin-secreting cells for the treatment of diabetes mellitus. Thus, islet progenitor cells in adult pancreatic ducts or in isolated islets of Langerhans have been induced to grow in culture, and their endocrine-like properties have been characterized. A proliferating beta-like cell line has been derived from tissue removed from a child with persistent hyperinsulinaemic hypoglycaemia of infancy and been engineered in culture to secrete insulin in response to glucose. Moreover, embryonic stem cells have been shown to adopt islet-like characteristics under defined culture conditions (see review by Docherty, 2001).

Further, investigations of pancreatic development have identified several genes involved in islet cell differentiation, many of which have been found to encode transcription factors, such as NeuroD (neurogenic differentiation factor) (Naya et al., 1995), Pdx-1 (pancreatic and duodenal homebox gene 1) (Offield et al., 1996), Is1-1 (islet factor 1) (Ahlgren et al., 1997), Pax4 (paired-box transcription factor 4) (Sosa-Pineda et al., 1997), Pax6 (paired-box transcription factor 6) (St-Onge et al., 1997), ngn3 (neurogenin3) (Gradwohl et al., 2000), homeobox gene of the NK-2 class, Nkx2.2 (Sussel et al., 1998) and HB9 (Li et al., 1999).

Identification of genes relevant to islet cell differentiation has led to proposed gene therapy mechanisms for the treatment of diabetes. For example, Ferber et al., 2000 describes a first generation (FG) adenoviral (Ad) vector having the PDX-1 gene as a potential composition for the treatment of type I diabetes. Systemic delivery of the composition to streptozotocin (STZ)-treated mice increased hepatic immunoreactive insulin content that was, in part, processed to mature biologically active mouse insulin 1 and 2, thereby ameliorating hyperglycemia in the diabetic mice. However, the immunoreactive insulin in the liver extracts was less than 1% of that in the pancreatic extracts. Further, because gene expression of FGAds is transient, the experiment was terminated after 8 days of treatment. Furthermore, FDAds are also highly hepatotoxic (O'Neal et al., 1998; Lozier et al., 1999).

More specifically, Pdx-1, also referred to as insulin promoter factor-1 (IPF-1), islet/duodenum homeobox-1 (IDX-1), somatostatin transactivating factor-1 (STF-1), insulin upstream factor-1 (IUF-1) and glucose-sensitive factor (GSF), is a transcription factor that is expressed in beta- and delta-cells of the islets of Langerhans and in dispersed endocrine cells of the duodenum. It is involved in regulating the expression of a number of key beta-cell genes as well as somatostatin. It also plays a pivotal part in the development of the pancreas and islet cell ontogeny. PDX-1 is known to be expressed early during development in cells of both exocrine and endocrine origin; later it becomes restricted primarily to beta-cells where it regulates the expression of beta-cell-specific genes and mediates the glucose effect on insulin gene transcription. PDX-1 is also known to be a key regulator of pancreatic morphogenesis and targeted disruption of the PDX-1 gene was described by Dutta et al. to lead to pancreatic agenesis in Pdx-1(−/−) homozygotes (Dutta et al., 2001). These studies involved expression of both wild-type and mutant PDX-1 transgenes and resulted in a corrected glucose intolerance in Pdx-1 heterozygotes mice. U.S. Pat. No. 6,210,960 to Habener et al. teaches treatment of diabetes involving administering to a patient afflicted with diabetes a recombinant IDX-1 polypeptide that transactivates the somatostatin promoter to treat the disease.

NeuroD, also referred to as BETA2/NeuroD, is a basic helix-loop-helix transcription factor and has been shown to play a role in the differentiation of neurons, olfactory cells, and neuroendocrine tissues. Further, NeuroD is known to be expressed in pancreatic endocrine cells during development and to regulate insulin gene expression. A polymorphism in exon 2 of NeuroD (Ala45Thr) has been reported to be associated with adult-onset type I diabetes in the Japanese population and the Danish population (Mochizuki et al., 2002; Hansen et al., 2000). Studies have demonstrated that the endocrine cells of the pancreas of BETA2/NeuroD-deficient mice undergoes massive apoptosis and, consequently, animals die of diabetes shortly after birth (Naya et al., 1997). It has also been demonstrated that BETA2/NeuroD-deficient mice restore the pancreatic beta-cells but not alpha-cell mass to a level comparable to wild-type (Huang et al., 2002). However, these restored beta-cells were found to lack the ability to form mature islets of Langerhans.

Neurogenin3 (ngn3) is also a basic helix-loop-helix (bHLH) transcription factor involved in islet cell differentiation and functions as a pro-endocrine factor in the developing pancreas. Ngn3 is detected along with early islet differentiation transcription factors Nkx6.1 and Nkx2.2, establishing that it is expressed in immature cells in the islet lineage (Schwitzgebel et al., 2000). Because ngn3 expression determines which precursor cells differentiate into islet cells, the signals that regulate ngn3 expression contribute to the mechanism that controls islet cell formation. Lee et al. observed in ngn3(−/−) mice that glucagon secreting A-cells, somatostatin secreting D-cells, and gastrin secreting G-cells are absent from the epithelium of the glandular stomach, whereas the number of serotonin-expressing enterochromaffin (EC) cells is decreased dramatically (Lee et al., 2002). Furthermore, the ngn3(−/−) mice displayed intestinal metaplasia of the gastric epithelium and, thus, the researchers concluded that ngn3 is required for the differentiation of enteroendocrine cells in the stomach and the maintenance of gastric epithelial cell identity. Huang et al. (2000) observed that overexpression of ngn3 induces the ectopic expression of BETA2/NeuroD in Xenopus embryos and stimulate the endogenous RNA of BETA2/NeuroD in endocrine cell lines.

Studies at a genetic level of the human ngn3 gene indicated that the ngn3 promoter drives transcription in all cell lines tested, including fibroblast cell lines and in transgenic animals, the promoter drives expression specifically in regions of ngn3 expression in the developing pancreas and gut with the addition of distal sequences greatly enhancing transgene expression (Lee et al., 2001). Based on their observations, the researchers concluded that ngn3 gene is activated by the coordinated activities of several pancreatic transcription factors and inhibited by HES1, an inhibitory bHLH factor activated by Notch signaling.

Betacellulin (BTC) is a beta-cell stimulating hormone growth factor that was originally isolated and identified from the conditioned medium from a murine pancreatic beta-cell carcinoma cell line (Kojima et al., 2002; U.S. Pat. No. 5,328,986). BTC is proteolytically processed from a larger membrane-anchored precursor and is a potent mitogen for a wide variety of cell types (Shing et al., 1993; Huotari et al., 1998). The peptide was identified as a member of the epidermal growth factor (EGF) family of peptide ligands that are characterized by a six-cysteine consensus motif (EGF-motif), which form three intra-molecular disulfide bonds that are crucial for binding the ErbB receptor family. The EGF signal transduction pathway is an important mediator of several cell functions and is based on the closely related tyrosine kinase receptor family. A variety of in vitro studies have identified BTC as an important factor in the growth and/or differentiation of pancreatic islet cells. The genomic structure of the mouse BTC (mBTC) gene was characterized by Lawson et al., 2002 and determined that the genomic polynucleotide contained six exons and five introns, an EGF-motif sequence encoded in exons 3 and 4, multiple transcription start sites, one poly(A) site, and several cis-acting regulatory elements in the promoter region (2.6 kb of 5′ flanking sequence).

The effect of betacellulin on regeneration of pancreatic beta-cells in 90%-pancreatectomized rats has also been described (Li et al., 2001). Post-pancreatectomy, Li et al. administered Wistar rats daily doses of betacellulin or saline for 10 days and observed in the betacellulin-treated rats a reduced plasma glucose response to i.p. glucose loading, an increase in plasma insulin concentration, beta-cell mass and insulin content. Thus, the researchers report that the administration of betacellulin improves glucose metabolism by promoting beta-cell regeneration in the pancreatectomized rats. Similar observations were described by Yamamoto et al. after a recombinant betacellulin was administered to mice having glucose intolerance induced by selective alloxan perfusion (Yamamoto et al., 2000).

Recent advances in the development of novel forms of insulin and improvements in islet transplantation have raised the bar for gene therapy for diabetes (Halban et al., 2001). A popular experimental approach in diabetes gene therapy is the transfer of a glucose-responsive insulin transgene to the liver of diabetic animals (Yoon et al., 2002). However, insulin production is highly complex and secretion is controlled mostly at the posttranscriptional and posttranslational levels. Insulin transgenes that are regulated at the transcriptional level cannot respond to the minute-to-minute changes in blood glucose during meals and exercise. Insulin gene transduction also fails to induce beta-cell-specific molecules, such as beta-cell-specific glucokinase, SUR1 and Kir6.2, and proinsulin-processing enzymes, that are required for the fine-tuning of insulin production. Furthermore, insulin produced as a result of insulin gene transfer is released from the target cell via the constitutive pathway, a process that is unregulated and unresponsive to the individual's second-to-second metabolic needs (Halban et al., 2001).

WO 02/29010 describes a method for obtaining in vitro mammal islet cells by preparing mammal pancreatic tissues by pancreas removal; dissociating the pancreatic tissues obtained into isolated pancreatic cells; optionally eliminating endocrine cells from the isolated pancreatic cells; inducing dedifferentiation of the isolated pancreatic cells into ductal precursor cells; and inducing redifferentiation of the ductal precursor cells into islet cells. The invention also concerns the use of the resulting islet cells for use in the treatment of pancreatic pathologies, particularly diabetes. However, islet grafts using such cells often are lost due to immune responses thereto.

The present invention is directed to a therapeutic regimen for the treatment of diabetes, more particularly type 1 diabetes, and fulfills a long-sought need in the art to treat diabetes without an adverse effect of heptatoxicity and without the problems experienced in the prior art such as, for example, with insulin gene transduction. To this end, compositions of the present invention provide an islet cell differentiation transcription factor to promote an increase in endogenous insulin levels. Certain embodiments of the present invention further comprise a helper dependent Ad (HDAd) vector that, in contrast to FGAd, provides prolonged transgene expression (Kim et al., 2001; Morral et al., 1998; Oka et al., 2001) and presents no inherent heptatoxicity (Kochanek, S., 1999) to the host. Administration of the compositions of the present invention provides to the diabetic patient an increase in insulin levels, an increase in insulin-producing cells and, thus, an increase in glucose tolerance in the patient.

### BRIEF SUMMARY OF THE INVENTION

The present invention is directed to compositions and methods that provide for the treatment of a diabetic patient. A non-exhaustive summary of the embodiments of the present invention are described as follows.

In one embodiment of the present invention, there is a method of treating a mammal for insulin-dependent diabetes comprising delivering to the mammal a composition comprising an effective amount of an islet cell differentiation transcription factor polypeptide or of a nucleic acid expressing the islet cell differentiation transcription factor polypeptide, wherein the factor promotes normalization of insulin level in the mammal to treat the insulin-dependent diabetes. In a specific embodiment, the delivering of the composition is in vivo. In another specific embodiment, the delivering of the composition to the mammal is further defined as introducing the composition into a somatic mammalian cell ex vivo; and delivering the cell comprising the composition to the individual. In a further specific embodiment, the composition is in a pharmaceutically acceptable diluent, and/or the islet cell differentiation transcription factor polypeptide is NeuroD, ngn3, Pax6, Pax4, Nkx2.2, Nkx6.1, Is1-1, or a combination thereof. In specific embodiments, the islet cell differentiation transcription factor is NeuroD or ngn3.

The methods of the present invention may further comprise administering a betacellulin polypeptide or a nucleic acid expressing the betacellulin polypeptide to the mammal. The betacellulin polypeptide and the islet cell differentiation factor polypeptide may be co-administered to the mammal, they may be in the same pharmaceutically acceptable diluent, the betacellulin polypeptide may be on the same molecule as the islet cell differentiation transcription factor polypeptide, and/or the nucleic acid expressing the betacellulin polypeptide may be on the same molecule as the nucleic acid expressing the islet cell differentation transcription factor polynucleotide.

Methods of the present invention may also further comprise administering a Pdx-1 polypeptide or a nucleic acid expressing the Pdx-1 polypeptide to the mammal. The Pdx-1 polypeptide and the islet cell differentiation factor polypeptide may be co-administered to the mammal. The nucleic acid may comprise an expression vector, such as a non-viral vector or a viral vector. The viral vector may be an adenoviral vector, a retroviral vector, a vaccinia viral vector, an adeno-associated viral vector, a polyoma viral vector, an alphaviral vector, a rhabdoviral vector or a herpes viral vector. In specific embodiments, the viral vector is an adenoviral vector, and the adenoviral vector may be helper dependent. The viral vector may be administered at between about 10¹¹ to about 10¹² viral particles. The viral vector may be administered at between about 1×10¹¹ to about 5×10¹¹ viral particles.

An expression vector of the present invention may further comprise a promoter operable in a eukaryotic cell, such as a tissue-specific promoter. Compositions may be administered systemically by continuous infusion or by intravenous injection. The composition may be injectable, and/or the composition may be administered intraperitoneally or intraportally.

In another embodiment of the present invention, there is a method of increasing an insulin level in a somatic cell comprising delivering to the cell a composition comprising an islet cell differentiation transcription factor polypeptide or a nucleic acid expressing the islet cell differentiation transcription factor polypeptide, wherein the presence of the polypeptide effects an increase in the insulin level in the cell. The delivering of the composition may be in vivo or in vitro. The somatic cell may be a hepatic cell, a pancreatic cell, a skeletal muscle cell, an adipose tissue cell, a stem cell, or a progenitor cell. A progenitor cell may be from skeletal muscle tissue, hepatic tissue, adipose tissue, or pancreatic tissue. The stem cell may be a hematopoietic cell, a pluripotent cell or a totipotent cell. In a specific embodiment, the stem cell is a pluripotent cell.

In specific embodiments of the present invention, the islet cell differentiation transcription factor polypeptide is NeuroD, ngn3, Pax6, Pax4, Nkx2.3, Nkx6.1, Is1-1 or a combination thereof.

In an additional embodiment of the present invention, there is a method of generating an insulin-producing cell comprising delivering to a somatic cell a composition comprising an islet cell differentiation factor polypeptide or a nucleic acid expressing the islet cell differentiation factor polypeptide, wherein the presence of the factor effects the generation of an insulin-producing cell from the somatic cell. A plurality of insulin-producing cells may be generated. In specific embodiments, at least one insulin-producing cell in the plurality is characterized by one or more secretory granules in the cytoplasm. In specific embodiments, at least one of the plurality of secretory granules comprises a diameter of about 300 nm to about 600 nm. In further specific embodiments, at least one of the plurality of secretory granules comprises an insulin polypeptide.

In another specific embodiment of the present invention, there is a therapeutic composition comprising an isolated islet cell differentiation transcription factor polypeptide and/or an isolated nucleic acid expressing the polypeptide. The islet cell differentiation transcription factor may be NeuroD, ngn3, Pax6, Pax4, Nkx2.3, Nkx6.1, Is1-1 or a combination thereof, and/or the composition may be in a pharmaceutically acceptable diluent. In specific embodiments, the nucleic acid is an expression vector. The expression vector may be a non-viral vector or a viral vector. The viral vector may be an adenoviral vector, a retroviral vector, a vaccinia viral vector, an adeno-associated viral vector, a polyoma viral vector, an alphaviral vector, a rhabdoviral vector or a herpes viral vector. The viral vector may be an adenoviral vector, and the adenoviral vector may be helper dependent.

In a specific embodiment, the composition comprises between about 10¹¹ to about 10¹² viral particles. The composition may further comprise an isolated betacellulin polypeptide or an isolated nucleic acid expressing the betacellulin polypeptide. The betacellulin nucleic acid may be an expression vector, such as a non-viral vector or a viral vector. The betacellulin viral vector may be an adenoviral vector, a retroviral vector, a vaccinia viral vector, an adeno-associated viral vector, a polyoma viral vector, an alphaviral vector, a rhabdoviral vector or a herpes viral vector.

An expression vector further comprises a promoter operable in a eukaryotic cell, such as a tissue-specific promoter.

In an additional embodiment of the present invention, there is an insulin-producing cell comprising a vector, the vector comprising nucleic acid sequence encoding an islet cell differentiation transcription factor. The cell may further comprise a vector comprising nucleic acid sequence encoding betacellulin. The cell may be in a pancreatic islet, and the pancreatic islet may be in a liver.

In another embodiment of the present invention, there is an insulin-producing cell generated by the method comprising obtaining a somatic cell; and transfecting said cell with a vector comprising nucleic acid sequence encoding an islet cell differentiation transcription factor, wherein upon said transfecting step said cell produces insulin. The insulin-producing cell may be further defined as a beta cell. The insulin-producing cell may be comprised in a pancreatic islet in vivo. The insulin-producing cell may be in the liver, and the islet may be in the liver.

In an additional embodiment of the present invention, there is a method of generating at least one pancreatic islet, comprising providing at least one somatic cell; and transfecting an effective amount of an islet cell differentiation transcription factor polypeptide or a nucleic acid expressing the islet cell differentiation transcription factor polypeptide into said cell, wherein upon said transfecting step said at least one pancreatic islet is generated. The pancreatic islet may be generated in liver tissue. The pancreatic islet may be generated in vitro or in vivo.

The somatic cell may be a hepatic cell, a pancreatic cell, a skeletal muscle cell, an adipose tissue cell, a stem cell, or a progenitor cell. The islet cell differentiation transcription factor may be NeuroD, ngn3, Pax6, Pax4, Nkx2.2, Nkx6.1, Is1-1, or a combination thereof.

In an additional embodiment of the present invention, there is use of a sequence for the treatment of type 1 or type 2 diabetes, said sequence having a region selected from the group consisting of SEQ ID NO:1 through SEQ ID NO:67, SEQ ID NO:79, and SEQ ID NO:83 through SEQ ID NO:93.

In another embodiment of the present invention, there is a composition comprising NeuroD polypeptide or a polynucleotide expressing a NeuroD polypeptide; and betacellulin polypeptide or a polynucleotide expressing a betacellulin polypeptide. The composition may further comprise a pharmaceutically acceptable diluent.

In an additional embodiment of the present invention, a composition may comprise ngn3 polypeptide or a polynucleotide expressing a ngn3 polypeptide; and further may comprise betacellulin polypeptide or a polynucleotide expressing a betacellulin polypeptide. The composition may further comprise a pharmaceutically acceptable diluent.

In a specific embodiment, there is a method of treating a mammal for insulin-dependent diabetes comprising delivering to the mammal in vivo or ex vivo a composition comprising an effective amount of an islet cell differentiation transcription factor polypeptide or of a nucleic acid expressing the islet cell differentiation transcription factor polypeptide, wherein the factor promotes normalization of insulin level in the mammal to treat the insulin-dependent diabetes

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIGS. 1A-1D illustrates graphically the effect of HDAd-Pdx-1 on the fasting serum glucose level (1A and 1C) and body weight (1B and 1D) of STZ mice in which either the BOS promoter (B-Pdx-1; 1A and 1B) or the PEPCK promoter (P-Pdx-1; 1C and 1D) was employed to control transgene expression;

FIGS. 2A-2C shows results of RT-PCR analysis of liver RNA for islet-specific hormones (2A), recombinant and endogenous Pdx-1 (2B), and other relevant factors (2C); lane 1, normal mouse pancreas RNA; lane 2, saline-treated non-diabetic liver; lane 3, saline-treated STZ mouse; lane 4, STZ mouse liver treated with 3×10¹¹ particles/mouse of B-Pdx-1; lane 5, STZ mouse liver treated with 3×10¹¹ particles/mouse of P-Pdx-1;

FIGS. 3A-3F shows fluorescence immunohistochemistry for insulin, Pdx-1 and trypsin for insulin-producing cells in the liver of HDAd-Pdx-1 treated STZ mice (3D-3F) as compared to STZ control (3A-3C);

FIGS. 4A-4C illustrate graphically the level of liver enzymes (4A and 4B) and bilirubin (4C) detected in HDAd-Pdx-1 treated STZ mice as compared to control mice;

FIGS. 5A-5B illustrates graphically the effect of HDAd gene therapy on the fasting serum glucose level and body weight in STZ mice, the different HDAd vectors delivering NeuroD (ND), BTC, or both, are as indicated, as are the dose in particles injected per mouse;

FIGS. 6A-6B illustrates graphically the effect of HDAd gene therapy on serum glucose (6A) and serum insulin (6B) levels in STZ mice;

FIGS. 7A-7B shows results of RT-PCR analysis of liver RNA taken from STZ mice treated with HDAd gene therapy (lanes 4-6) as compared to control mice (lanes 1-3) and controls; lane 1, normal mouse pancreas; lane 2, saline-treated nondiabetic liver; lane 3, saline-treated STZ diabetic liver; lane 4, BTC-treated STZ diabetic liver; lane 5, NeuroD-treated STZ diabetic liver; lane 6, NeuroD+BTC-treated STZ diabetic liver;

FIGS. 8A-8P shows the results of fluorescence immunohistochemistry of insulin-producing cells in the liver of STZ mice 4 months post-treatment with HDAd gene therapy;

FIGS. 9A-9D shows electron micrographs of the insulin-producing cells in the liver of STZ mice post-treatment with HDAd-NeuroD plus betacellulin (BTC) gene therapy;

FIG. 10 illustrates exemplary helper-dependent adenoviral vectors useful in the present invention;

FIG. 11 illustrates the effect of ngn3 gene therapy on glucose levels in treated mice; and

FIG. 12 shows an intraperitoneal glucose tolerance test in mice treated with ngn3 gene therapy in nondiabetic, STZ diabetic and ngn3 gene therapy-treated STZ diabetic mice.

### DETAILED DESCRIPTION OF THE INVENTION

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can be, and has been, used as a recipient for vectors. A host cell may be “transfected” or “transformed,” which refers to a process by which exogenous nucleic acid is transferred or introduced into the host cell. A transformed cell includes the primary subject cell and its progeny.

The term “delivering” as used herein is defined as bringing to a destination, providing, and includes administering, as for a therapeutic purpose.

The term “delivery vehicle” as used herein is defined as an entity which is associated with transfer of another entity. Said delivery vehicle is selected from the group consisting of an adenoviral vector, a retroviral vector, a lentiviral vector, an adeno-associated vector, a plasmid, a liposome, a protenoid, an emulsion, a colloidal suspension, a nucleic acid, a peptide, a lipid, a carbohydrate, a natural or a synthetic polymer and a combination thereof.

The term “diabetes” as used herein is defined as a disease resulting either from an absolute deficiency of insulin due to a defect in the biosynthesis or production of insulin, or a relative deficiency of insulin in the presence of insulin resistance, i.e., impaired insulin action, in an organism. The term “diabetic patient” as used herein refers to a human who has type 1 diabetes, i.e., absolute insulin deficiency, or type 2 diabetes, i.e., relative insulin deficiency in the presence of insulin resistance. The diabetic patient thus has absolute or relative insulin deficiency, and displays, among other symptoms and signs, eleveated blood glucose concentration, presence of glucose in the urine and excessive discharge of urine.

The term “first phase insulin response” as used herein refers to a rapid and transient burst of insulin secretion in response to an abrupt increase of glucose level that subsides within about 10 minutes in most individuals.

The term “hepatotoxicity” as used herein refers to a) serum liver enzyme elevation, i.e., elevated serum concentrations of alanine aminotransferase (ALT) and/or aspartate aminotransferase AST), b) serum bilirubin concentration elevation; and/or c) inflammatory cell (leukocyte) infiltration in the liver, such as revealed by histology. In preferred embodiments, a vector of the present invention comprising an islet cell differentiation transcription factor has substantially no hepatotoxicity, i.e., in response to the treatment liver enzyme levels do not increase more than about three times the upper limit of normal.

The term “increases” as used herein is defined as adding to, augmenting, multiplying, propagating or to make greater in any respect a desirable result. The increase may be complete or may be partial.

The term “islet cell differentiation transcription factor” as used herein is defined as any molecule, either a polypeptide or a nucleic acid expressing the polypeptide, that is involved in islet cell differentiation by functioning as a transcription factor. The skilled artisan is aware that genes are regulated by transcription factors, which bind to DNA regulatory elements near a coding sequence. It is contemplated that the transcription factor may also participate in additional mechanisms directed to development, metabolism or the like. In specific embodiments, the islet cell differentiation transcription factor includes, but is not limited to, Pdx-1, NeuroD, Pax6, Pax4, Nkx2.2, Nkx6.1, Is1-1, or ngn3. Furthermore, multiple homologs or similar sequences can exist in a mammal, and these can easily be identified by standard means in the art, such as by searching the National Center for Biotechnology Information's GenBank database.

The term “islet cell growth factor” as used herein is defined as a protein, polypeptide, or peptide molecule that functions to induce a specific target cell, e.g., islet cell, to grow and/or differentiate. In specific embodiments, the islet cell growth factor is betacellulin, which is often abbreviated as BTC.

The term “normalization of insulin level” as used herein regards plasma glucose in a treated diabetic mouse being the same before and after glucose challenge and/or feeding as in a non-diabetic mouse.

The term “second phase insulin response” as used herein refers to a slow and progressive increase of insulin secretion, which continues for the duration of the exposure to high glucose concentration up to about several hours.

The term “somatic cell” as used herein refers to a cell of an animal body other than egg or sperm. Where a plurality of somatic cells are contemplated, more than one type of somatic cell is suitable, such as in a mixture of hepatocytes and mature hepatic (liver) cells. A non-limiting example of a plurality of different somatic cells includes a mixture of a hepatic stem cell, a progenitor cell such as a pluripotent stem cell and a mature liver cell. One of ordinary skill in the art is aware of other variations that are within the scope of the present invention.

The term “stem cell” as used herein refers to an undifferentiated, primitive cell with the ability both to multiply and to differentiate into a specific kind or type of cell. Thus, in the present invention the stem cell enables the growth and generation of specialized cells or tissue in vitro, which are used to treat a disease in vivo or by utilizing ex vivo methods. In specific embodiments, the stem cell is a “pluripotent stem cell”, which refers to an undifferentiated cell that is capable of developing or differentiating into multiple cell and/or tissue types of an organism. In other specific embodiments, the stem cell is “totipotent”, which refers to an undifferentiated cell that is capable of developing into a complete organism. Other types of stem cells contemplated include hematopoietic cells, which are the blood-producing cells in the bone marrow, neuronal stem cells, and/or stems cells isolated from the liver, muscle or fat tissue.

The term “STZ mouse” or “STZ mice,” as used herein, refers to a mouse or a plurality of mice, respectively, standard in the art, that have been treated with streptozotocin (STZ) to induce a diabetic state that mimics type 1 diabetes in a human.

The terms “therapeutically effective amount” or interchangeably “effective amount” as used herein refer to that amount sufficient to detectably and repeatedly improve, increase, prevent, treat, effect, promote, enhance, induce or ameliorate a desired result. Further, an effective amount of the pharmaceutical composition, generally, is defined as that amount sufficient to detectably and repeatedly ameliorate, reduce, minimize or limit the extent of the disease or its symptoms. In some embodiments the amount provides elimination, eradication, or cure of disease.

### The Present Invention

The present invention is directed to methods and compositions that induce and promote the production of insulin and/or the development of pancreatic islets in vitro or in vivo, such as but not limited to the liver. In further embodiments, the methods and compositions are useful in the treatment of diabetes mellitus in vivo or by utilizing an ex vivo method by providing a partial or complete reversal of the diabetic state in a mammal. The type of diabetes to be treated in the present invention may be of any type, including Type 1 and Type 2. In some other embodiments, the treated individual has type 1 or type 2 diabetes, i.e., elevated blood glucose, but may or may not exhibit symptoms as of yet.

Applicants' observed that adenoviral-mediated overexpression of islet cell differentiation transcription factors and/or islet cell growth factors led to the rapid induction of insulin production in situ. A concomitant increase in proinsulin, glucagon, somatostatin, and pancreatic polypeptide levels, as well as the presence of pancreatic islet structures in the liver, also resulted from administration in vivo of the inventive compositions. These characteristics indicate that islet cell differentiation transcription factors, such as, for example, ngn3 and NeuroD, either alone or in combination with other islet cell differentiation transcription factors or islet cell growth factors, have broad therapeutic, prognostic and diagnostic potential as a therapeutic agent for the treatment of diabetes mellitus.

Thus, the present invention concerns the triggering of cells in the liver to produce insulin. In some embodiments, the insulin produced is in the form of phenotypically normal insulin granules inside vesicles, as opposed to cytosolic insulin. In one embodiment, a therapeutic gene product comprising an islet cell differentiation transcription factor, which may be delivered in the form of a nucleic acid, is delivered to a diabetic individual. Delivery may be systemic or local in nature. Once in the liver, the therapeutic gene product facilitates the production of beta cells to make insulin and, in some embodiments, islet cells that produce other hormones such as glucagon, somatostatin, pancreatic polypeptide, and/or others, which play a role in regulating insulin production and release, as well as directly regulating glucose metabolism. The cells that develop into beta cells to produce insulin may be of any kind so long as they are at least capable of producing insulin. The new beta cells may exhibit other characteristics related to endogenous beta cells, and these are described elsewhere herein. In specific embodiments, the cells that develop into insulin-producing cells are stem cells, such as liver stem cells, bone marrow stem cells, fat stem cells, muscle stem cells, and so forth.

In an alternative embodiment, for example by ex vivo therapy, cells are removed from an individual, such as a diabetic individual, the therapeutic gene or gene product is delivered to the removed cells, and the resultant insulin-producing beta cells are transferred to a diabetic individual. In preferred embodiments, the cells are removed from the same diabetic individual to be treated. This is a powerful technique given that it does not elicit an immune system response, as in standard cell transplant therapies. That is, the present invention advantageously boosts the body to use its own cells. In the embodiment wherein a diabetic individual's own cells are used for transplantation, it also eliminates the challenge of obtaining a matching cell type from a donor. However, in the embodiments wherein at least one cell from a donor is transplanted into another individual, a skilled artisan recognizes that standard immunosuppressive measures should be taken (see, for example, Shapiro et al., 2000).

In some embodiments of the present invention, the beta cells produced are comprised in complete islets, which are known in the field to be densely packed collections of polypeptide hormone-producing cells, all of which are involved in metabolic regulation. The islet cells may be comprised of the following: 1) beta cells that produce insulin; 2) alpha cells that produce glucagon; 3) delta cells (or D cells) that produce somatostatin; and/or F cells that produce pancreatic polypeptide. The polypeptide hormones (insulin, glucagon, somatostatin and pancreatic polypeptide) inside these cells are stored in secretary vesicles in the form of secretory granules.

Furthermore, a skilled artisan recognizes that exocrine gene expression is undesirable when restoring insulin production in the liver. The liver has no pancreatic ducts, and insulin will simply be secreted into the bloodstream in the liver (similar to the insulin produced by the pancreas, which also is simply secreted into the bloodstream). When trypsin (a digestive enzyme) is produced, in the absence of pancreatic ducts that normally deliver the digestive enzyme into the lumen of the gut, the trypsin would proteolyze (digest) proteins once it is secreted into the bloodstream. Further, if the trypsin is not confined to special vesicles in the liver cells that produce it, it will likely digest and kill the cells that produce the enzyme. As a consequence, Pdx1 delivered by the HDPdx1, described in certain Examples herein, appears to be a suicidal gene. Without desiring to be bound by any theory, this may account for the very short duration of the hypoglycemic response after HDPdx1 treatment: as insulin and trypsin are produced by these cells, the insulin lowers the blood glucose, but almost immediately afterwards, the insulin (and trypsin) producing cells die as they are digested by the trypsin. There is no more insulin production, and blood glucose goes up again. Furthermore, the dead cells induce a severe inflammatory response, causing severe hepatitis, which uniformly accompanies treatment using HDPdx1. Thus, in specific embodiments, insulin production and not trypsin (or any other exocrine or digestive enzyme) production is desirable by the present invention.

I. Islet Cell Differentiation Transcription Factors

In certain embodiments, the present invention is directed to administration of an effective amount of an islet cell differentiation transcription factor polypeptide to treat insulin-dependent diabetes. In certain embodiments, the islet cell differentiation factor is provided as at least one polypeptide molecule. In other embodiments, the islet cell differentiation factor is provided as at least one polynucleotide molecule.

In specific embodiments, the present invention involves administering or delivering an effective amount of a NeuroD polypeptide or protein. A skilled artisan is aware that nucleic acid and/or amino acid sequences are available, such as at the National Center for Biotechnology Information's GenBank database. The NeuroD polypeptide of the present invention comprises an amino acid sequence of SEQ ID NO:1, which corresponds to gene accession no. AAA93480 or its homologs, including, but not limited to (and followed by their corresponding GenBank Accession No.), SEQ ID NO:2 (AAB37576); SEQ ID NO:3 (Q13562); SEQ ID NO:4 (NP_{—}062091); SEQ ID NO:5 (P79765); SEQ ID NO:6 (Q91616); SEQ ID NO:7 (Q64289); SEQ ID NO:8 (Q60867); SEQ ID NO:9 (Q60430); SEQ ID NO:10 (XP_{—}002573); SEQ ID NO:11 (NP_{—}571053); SEQ ID NO:12 (AAB88820); SEQ ID NO:13 (AAB70529); SEQ ID NO:14 (149338); SEQ ID NO:15 (JC4688); SEQ ID NO:16 (151687); SEQ ID NO:17 (AAG09285); SEQ ID NO:18 (NP_{—}002491); SEQ ID NO:19 (BAA77569); SEQ ID NO:20 (BAA76603); SEQ ID NO:21 (BAA87605); SEQ ID NO:22 (BAA81821); SEQ ID NO:23 (AAD23995); SEQ ID NO:24 (BAA11558); SEQ ID NO:25 (AAD19609); SEQ ID NO:26 (AAC79425); SEQ ID NO:27 (AAC59675); SEQ ID NO:28 (AAC52204); SEQ ID NO:29 (AAC52203); SEQ ID NO:30 (AAC51318); SEQ ID NO:31 (AAC26058); SEQ ID NO:32 (CAA70784); SEQ ID NO:33 (AAC12470); SEQ ID NO:34 (AAC12469); SEQ ID NO:35 (AAC12468); SEQ ID NO:36 (AAC12467); SEQ ID NO:37 (AAC12466); SEQ ID NO:38 (AAC12462); SEQ ID NO:39 (AAC12461); SEQ ID NO:40 (BAA11931); SEQ ID NO:41 (AAB38744); SEQ ID NO:42 (AAB37575); SEQ ID NO:43 (2111505A); SEQ ID NO:44 (AAA79702), or SEQ ID NO:79 (AAA79702). Examples of NeuroD polynucleotides useful in the present invention include SEQ ID NO:170 (U50822), SEQ ID NO:171 (U28888), and SEQ ID NO: 192 (U28068).

In other specific embodiments, the present invention involves administering or delivering an effective amount of a ngn3 polypeptide or protein. The ngn3 polypeptide of the present invention comprises an amino acid sequence of SEQ ID NO:45, which corresponds to gene accession no. AAK15022, or its homologs including but not limited to, SEQ ID NO:46 (AAK50058); SEQ ID NO:47 (Q9Y4Z2); SEQ ID NO:48 (XP_{—}122040); SEQ ID NO:49 (XP_{—}167394); SEQ ID NO:50 (AAG09438); SEQ ID NO:51 (NP_{—}066279); SEQ ID NO:52 (CAA70366); SEQ ID NO:53 (CAB45384); or SEQ ID NO:54 (AAC53029). Examples of ngn3 polynucleotides useful in the present invention include SEQ ID NO:172 (AF234829) and SEQ ID NO:173 (AF364300).

In further specific embodiments, the present invention involves administering or delivering an effective amount of a Pdx-1 polypeptide or protein. The Pdx-1 polypeptide or protein is administered or delivered in a different or in the same delivery vehicle as the islet cell differentiation transcription factor selected from the group consisting of NeuroD, ngn3, Pax4, Pax6, Nkx2.2, Nkx6.1 or Is1-1. The Pdx-1 polypeptide of the present invention comprises an amino acid sequence of SEQ ID NO:55, which corresponds to GenBank Accession No. AAA88820, or its homologs including, but not limited to, SEQ ID NO:56 (NP_{—}032840); SEQ ID NO:57 (NP_{—}571518); SEQ ID NO:58 (NP_{—}074043); SEQ ID NO:59 (P70118); SEQ ID NO:60 (P52947); SEQ ID NO:61 (P52946); SEQ ID NO:62 (P52945); SEQ ID NO:63 (XP_{—}124700); SEQ ID NO:64 (BAB32045); SEQ ID NO:65 (NP_{—}032840); SEQ ID NO:66 (AAB88463); or SEQ ID NO:67 (AAB18252). Examples of useful Pdx-1 polynucleotides in the present invention include SEQ ID NO:190 (U35632), SEQ ID NO:191 (NM_{—}008814), or SEQ ID NO:194 (XM_{—}124700).

In other further specific embodiments, the present invention involves administering or delivering an effective amount of a Pax4 polypeptide or protein. The Pax4 polypeptide of the present invention comprises an amino acid sequence of SEQ ID NO:83, which corresponds to GenBank Accession No. AAD02289, or its homologs including, but not limited to, SEQ ID NO:84 (AAF14073). Examples of Pax4 polynucleotides useful in the present invention include SEQ ID NO:176 (AF043978) or SEQ ID NO:177 (AF104231).

In other further specific embodiments, the present invention involves administering or delivering an effective amount of a Pax6 polypeptide or protein. The Pax6 polypeptide of the present invention comprises an amino acid sequence of SEQ ID NO:85, which corresponds to GenBank Accession No. AAK95849, or its homologs including, but not limited to, SEQ ID NO:86 (CAC83748). Examples of Pax6 polynucleotides useful in the present invention include SEQ ID NO:174 (AY047583) or SEQ ID NO:175 (AJ307468).

In other further specific embodiments, the present invention involves administering or delivering an effective amount of a Nkx2.2 polypeptide or protein. The Nkx2.2 polypeptide of the present invention comprises an amino acid sequence of SEQ ID NO:87, which corresponds to gene accession no. AAC83132, or its homologs including, but not limited to, for example, SEQ ID NO:88 (AAK93795). Examples of Nkx2.2 polynucleotides useful in the present invention include SEQ ID NO:178 (AF019414); SEQ ID NO:179 (AF019415); SEQ ID NO:180 (AY044657); and/or SEQ ID NO:181 (AY044658).

In other further specific embodiments, the present invention involves administering or delivering an effective amount of a Nkx6.1 polypeptide or protein. The Nkx6.1 polypeptide of the present invention comprises an amino acid sequence of SEQ ID NO:89, which corresponds to GenBank Accession No. AAD11962, or its homologs including, but not limited to, SEQ ID NO:90 (AAK37567). Examples of Nkx6.1 polynucleotides useful in the present invention include SEQ ID NO:182 (U66797); SEQ ID NO:183 (U66798); SEQ ID NO:184 (U66799); and/or SEQ ID NO:185 (AF357883).

In other further specific embodiments, the present invention involves administering or delivering an effective amount of a Is1-1 polypeptide or protein. The Is1-1 polypeptide of the present invention comprises an amino acid sequence of SEQ ID NO:91, which corresponds to GenBank Accession No. NP_{—}002193, or its homologs including, but not limited to, SEQ ID NO:92 (NP_{—}067434) and/or SEQ ID NO:93 (XP_{—}122631). Examples of Is1-1 polynucleotides useful in the present invention include SEQ ID NO:186 (NM_{—}002202), SEQ ID NO:187 (BC017027), and SEQ ID NO:193 (XM_{—}122631).

In other further specific embodiments, the present invention involves administering or delivering an effective amount of a BTC polypeptide or protein. The BTC polypeptide or protein is administered or delivered in a different or in the same delivery vehicle as the islet cell differentiation transcription factor selected from the group consisting of NeuroD, ngn3, Pax4, Pax6, Nkx2.2, Nkx6.1 or Is1-1, alone or together with the Pdx-1 polypeptide or protein. The BTC polypeptide of the present invention comprises an amino acid sequence of SEQ ID NO:68, which corresponds to GenBank Accession No. XP_{—}172810, or its homologs including, but not limited to, SEQ ID NO:69 (AAA40511); SEQ ID NO:70 (NP_{—}071592); SEQ ID NO:71 (AAM21214); SEQ ID NO:72 (XP_{—}124577); SEQ ID NO:73 (NP_{—}031594); SEQ ID NO:74 (NP_{—}001720); SEQ ID NO:75 (BAA96731); SEQ ID NO:76 (AAF15401); SEQ ID NO:77 (AAB25452); or SEQ ID NO:78 (AAA40511). In other embodiments, the BTC polypeptide is a full-length protein, i.e., preprotein or BTC precursor, that has not been proteolytically cleaved such as in amino acid sequences of SEQ ID NO:80 (AAA40511); SEQ ID NO:81 (Q05928); or SEQ ID NO:82 (P35070). Examples of betacellulin polynucleotides useful in the present invention include SEQ ID NO:188 (XM_{—}172810) or SEQ ID NO:189 (L08394).

The term “homolog” refers to a biologically functional equivalent polypeptide or protein, as defined in the sections titled Variants of Proteinaceous Compositions and Nucleic Acids, and a structurally equivalent polypeptide or protein, in that the amino acid sequences of interest have about 70%, about 75%, about 80%, about 85%, about 90%, about 95%, to about 99% of amino acids that are identical or functionally equivalent (functional equivalence is discussed further in section titled, Functional Aspects).

I. Proteinaceous Compositions

The present invention involves proteins, polypeptides and/or peptides. In specific embodiments, the protein, polypeptide or peptide is an islet cell differentiation transcription factor. In other specific embodiments, the protein, polypeptide or peptide is an islet cell growth factor. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein. The invention includes those lengths of contiguous amino acids of any sequence discussed herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, delivered to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least a part of an antibody, for example, an antibody against a molecule expressed on a cell's surface, to allow an islet cell differentiation transcription factor composition to be targeted to the cell. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated.

A. Functional Aspects

When the present application refers to the function or activity of an islet cell differentiation transcription factor polypeptide, it is meant that the molecule in question functions to bind to a nucleic acid sequence, e.g., DNA, to promote the synthesis of a complementary nucleic acid molecule, e.g., RNA. Determination of which molecules possess this activity may be achieved using assays familiar to those of skill in the art.

When the present application refers to the function or activity of BTC, it is meant that the molecule in question functions as a ligand for an EGF (epidermal growth factor) receptor protein, binds to heparin and participates in the growth and differentiation mechanisms of islet cells in a pancreas. Determination of which molecules possess this activity may be achieved using assays familiar to those of skill in the art.

In terms of functional equivalents, the skilled artisan understands that inherent in the definition of a biologically-functional equivalent protein, polypeptide or peptide, is the concept of a limit to the number of changes that may be made within a defined portion of a molecule that still result in a molecule with an acceptable level of equivalent biological activity. Biologically-functional equivalent proteins, polypeptides or peptides are thus defined herein as those proteins, polypeptides or peptides in which certain, not most or all, of the amino acids may be substituted. In particular, where small proteins, polypeptides or peptides are concerned, less amino acids may be changed. Of course, a plurality of distinct proteins, polypeptides or peptides with different substitutions may easily be made and used in accordance with the invention.

It is also well understood that where certain residues are shown to be particularly important to the biological or structural properties of a protein, polypeptide or peptide, i.e., residues in the active site of an enzyme, or in the DNA binding region, such residues may not generally be exchanged. This is the case in the present invention, where residues shown to be necessary for increasing insulin levels or inducing generation of insulin-producing cells should not generally be changed.

While discussion has focused on functionally equivalent proteins, polypeptides or peptides arising from amino acid changes, it will be appreciated that these changes may be effected by alteration of the encoding DNA, taking into consideration also that the genetic code is degenerate and that two or more codons may encode the same amino acid. A table of amino acids and their codons is presented below for use in such embodiments, as well as for other uses, such as in the design of probes and primers and the like.

B. Variants of Proteinaceous Compositions

Amino acid sequence variants of the polypeptides and peptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the islet cell differentiation transcription factor polypeptide/protein/peptide or the islet cell growth factor polypeptide/protein/peptide provided the biological activity of the protein is maintained. (see Table 1, below for a list of functionally equivalent codons).

TABLE 1
Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (31 0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within +1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of an islet cell differentiation transcription factor molecule, an islet cell growth factor molecule or a linking moiety, but with altered and even improved characteristics.

1. Fusion Proteins

A specialized kind of insertional variant is the fusion protein. This molecule generally has all or a substantial portion of the native molecule, linked at the N- or C-terminus, to all or a portion of a second polypeptide. In the present invention, a fusion may comprise a islet cell differentiation transcription factor sequence and/or the islet cell growth factor sequence together with a linking moiety or a reporter (detectable) molecule. In other examples, fusions employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

2. Synthetic Peptides

The present invention describes islet cell differentiation transcription factor polypeptides and/or islet cell growth factor peptides for use in various embodiments of the present invention. Specific peptides are assayed for their abilities to elicit an immune response. In specific embodiments that the peptides are relatively small in size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. For example, in specific embodiments a BTC polypeptide or peptide is administered or delivered. The BTC polypeptide is preferably in the mature form, e.g. proteolytically processed in vivo or in vitro, which may be achieved by methods well known in the art such as, directly administering or delivering the mature BTC polypeptide to the host organism or cell, or alternatively, administering or delivering the BTC as a nucleic acid expressing the mature BTC gene product.

Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

Alternatively, recombinant DNA technology may be employed wherein a nucleotide sequence which encodes a peptide of the invention is inserted into an expression vector, transformed or transfected into an appropriate host cell and cultivated under conditions suitable for expression.

The compositions of the invention may include a peptide comprising an islet cell differentiation transcription factor polypeptide that has been modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity. Further, the compositions of the present invention may comprise a ligand that is covalently attached to the transcription factor by way of a linking moiety. The ligand is a polypeptide that may also be modified to render it biologically protected.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

3. In Vitro Protein Production

In certain embodiments, the composition of the present invention is administered ex vivo. Such methods involve preparing a culture of a plurality of somatic cells, i.e., progenitor cells, comprising the recombinant islet cell differentiation transcription factor polypeptide; and administering or delivering the cells to host. In specific embodiments involving a nucleic acid expressing the polypeptide, the nucleic acid further comprises an expression vector, such as a viral vector. The somatic cell is transduced with the composition, and following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogene product, and more specifically, an protein having immunogenic activity. Other examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

II. Methods of Use

The intravenous administration of the exemplary HDAds expressing an islet cell differentiation transcription factor and/or an islet cell growth factor produced and sustained normalization of blood glucose in diabetic mammals, indicating the development of glucose-sensing mechanisms, e.g., in the islet structures. The treatment comprised a single islet cell differentiation transcription factor or the transcription factor in combination with at least a second islet cell differentiation transcription factor that is different from the first, and/or with an islet cell growth factor. Further, the treatment provided pancreatic islet structures in the liver of the treated mammals and immunoreactive insulin, proinsulin, glucagon and pancreatic polypeptide detection by histological examination thereof. It is known in the art that the pancreatic polypeptide is an agonist of neuropeptide Y5 receptor (Cabrele, et al., 2000). The proinsulin was processed to insulin in the newly formed or generated islet cells, thereby indicating the presence of the appropriate proinsulin processing enzymes. Thus, the in vivo or ex vivo therapy methods and compositions of the present invention provide a powerful regime for the treatment of diabetes mellitus.

A. Therapeutic Formulations and Routes of Administration

The present invention discloses the compositions and methods involving in increase in insulin levels, an increase in insulin-producing cells and, thus, a treatment of diabetes. Where clinical applications are contemplated, it will be necessary to prepare the compositions of the present invention as pharmaceutical compositions, i.e., in a form appropriate for in vivo and/or ex vivo applications. Generally, this will entail preparing compositions that are essentially free of pyrogens, as well as other impurities that could be harmful to humans or animals.

1. Preparation Methods

The compounds of the present invention include a composition comprising an islet cell differentiation transcription factor molecule and in some embodiments, an islet cell growth factor molecule (i.e. polypeptide, protein or peptide, each used interchangeably herein) or more than one islet cell differentiation transcription factor molecule. The islet cell differentiation transcription factor or a composition thereof may be linked, or operatively attached, to the islet cell growth factor or the additional islet cell differentation transcription factor by either chemical conjugation (e.g., crosslinking) or through recombinant DNA techniques to produce the compound.

Where recombinant DNA techniques are utilized nucleic acid expressing the islet comprises cell growth factor molecule is employed. Further the nucleic acid comprises an expression vector, which is a non-viral vector or a viral vector. In specific embodiments involving the use of viral vector, the viral vector is an adenoviral vector, a retroviral vector, a lentiviral vector, a vaccinia viral vector, an adeno-associated viral vector, a polyoma viral vector, an alphaviral vector, a rhabdoviral vector or a herpes viral vector. It is prefered that the viral vector is an adenoviral vector, and in further embodiments, the adenoviral vector is helper dependent. The effective amount of the viral vector is contemplated at between about 10¹¹ viral particles per kilogram to about 10¹³ viral particles per kilogram body weight, or more specifically, at between about 1×10¹¹ to about 5×10¹³ viral particles per kilogram body weight. These amounts are administered systemically, subcutaneously, intravenously, intraportally, intrahepatic arterially, intraperitoneally, by means of continuous infusion or by direct injection.

2. Formulations and Administrations

One will generally desire to employ appropriate salts and buffers to render delivery vectors and compositions stable and allow for uptake by target cells. Buffers also will be employed when recombinant cells are introduced into a patient. Aqueous compositions of the present invention comprise an effective amount of the viral composition to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” or “pharmaceutically acceptable diluent” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intralesional, intramuscular, intraportal, intra-hepatic arterial, intraperitoneal or intravenous. Such compositions would normally be administered as pharmaceutically acceptable compositions, described supra.

The active compounds may be administered via any suitable route, including parenterally or by direct injection, i.e., into a portal vein of the mammal, or inhalation. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The present invention is administered using a variety of mechanisms including, intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraportally, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference in its entirety).

Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1 to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10 to about 95% of active ingredient, preferably about 25 to about 70%.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5.

In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

In certain embodiments, active compounds may be administered orally. This is contemplated to be useful as many substances contained in tablets designed for oral use are absorbed by mucosal epithelia along the gastrointestinal tract.

Also, if desired, the peptides, polypeptides, proteins, and other agents may be rendered resistant, or partially resistant, to proteolysis by digestive enzymes. Such compounds are contemplated to include chemically designed or modified agents; dextrorotatory peptides; and peptide and liposomal formulations in time release capsules to avoid peptidase and lipase degradation.

For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like.

Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations. In certain embodiments, extensive dialysis is employed to remove undesired small molecular weight molecules and/or lyophilized for more ready formulation into a desired vehicle.

Upon formulation, the compounds will be administered in a manner compatible with the dosage formulation and in such amount as is therapeutically effective. The formulations are easily administered in a variety of dosage forms, as described herein.

Typically, compositions of the present invention are prepared as injectables. Either as liquid solutions or suspensions: solid forms suitable for solution in, or suspension in, liquid prior to injection may also be prepared. The preparation may also be emulsified. The active ingredient (i.e., recombinant molecule or cell) is often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, or adjuvants which enhance the effectiveness of the vaccines.

Direct injection may be conventionally administered parenterally, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1 to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10 to about 95% of active ingredient, preferably about 25 to about 70%.

The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per dose. Suitable regimes for initial administration and subsequent administrations, if necessary, are also variable, but are typified by an initial administration followed by subsequent administrations of the therapeutic composition, if necessary. The course of the therapy may be followed by assays for a level of the transgene expression or for a level of endogenous insulin. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

“Unit dose” is defined as a discrete amount of a therapeutic composition dispersed in a suitable carrier. For example, in accordance with the present methods, viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³, 10¹⁴ or 10¹⁵ pfu or viral particles. Particle doses may be somewhat higher (10 to 100-fold) due to the presence of infection-defective particles.

In this connection, sterile aqueous media which can be employed will be known to those of skill in the art in light of the present disclosure. For example, a unit dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the present invention is directed at the treatment of human diabetes. A variety of different routes of administration are contemplated. For example, a classic and typical therapy will involve systemic, subcutaneous injection of the mammal. The injections may be single or multiple; where multiple, injections are made at about the same or different locations of the mammal. Alternatively, targeting the liver vasculature by direct, local or regional intra-portal injection are contemplated. The lymphatic systems, including regional lymph nodes, present another likely target given the potential for metastasis along this route. Further, systemic injection may be preferred when specifically targeting an enhancement in the proliferation of the islet cells or in the treatment using an islet cell growth factor polypeptide/peptide.

Another method for achieving treatment is via catheterization of the portal vein, thereby permitting continuous perfusion with composition over extended periods. This method is suitable for a patient that has undergone an islet cell graft surgery and is treated with the composition for a post-operative period.

3. Combination Therapy

In many therapies, it will be advantageous to provide more than one functional therapeutic agent. Such “combined” therapies may have particular importance in treating aspects of multidrug resistant (MDR) cancers and in antibiotic resistant bacterial infections. Thus, one aspect of the present invention utilizes a composition comprising an islet cell differentiation transcription factor polypeptide or a nucleic acid expressing the islet cell differentiation transcription factor polypeptide, while a second therapy, either targeted or non-targeted, also is provided.

Alternatively, the present invention utilizes a viral composition comprising a viral vector encoding a islet cell differentiation transcription factor polypeptide to deliver therapeutic compounds for treatment of diseases, while a second therapy, either targeted or non-targeted, also is provided. Such second thereapy contemplated includes the targeted or non-targeted delivery of an islet cell growth factor as the second therapeutic agent.

The non-targeted treatment may precede or follow the targeted agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the targeted agent is “A” and the non-targeted agent is “B”, as exemplified below, however, other combinations are contemplated:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

To generate islet cells, promote cell growth, increase insulin levels, or otherwise reverse the diabetic phenotype of pancreatic cells, using the methods and compositions of the present invention, one would generally contact a “target” cell with a targeting agent/therapeutic agent and at least one other agent; these compositions would be provided in a combined amount effective achieve these goals. Target cells useful according to the invention will include, but not be limited to, pancreatic cells, e.g., non-islet pancreatic cells, pancreatic islet cells, islet cells of the beta-cell type, non-beta-cell islet cells, and pancreatic duct cells. These cell types may be isolated according to methods known in the art for ex vivo manipulation. See, e.g., Githens, 1988, Jour. Pediatr. Gastroenterol. Nutr. 7:486; Warnock et al., 1988, Transplantation 45:957; Griffin et al., 1986, Brit. Jour. Surg. 73:712; Kuhn et al., 1985, Biomed. Biochim. Acta 44:149; Bandisode, 1985, Biochem. Biophys. Res. Comm. 128:396; Gray et al., 1984, Diabetes 33:1055, all of which are hereby incorporated by reference. Also contemplated as target cells are cell mixtures comprising any of a hepatocyte, a mature liver cell, a progenitor cell including a stem cell, a pluripotent stem cell, a totipotent stem cell, a hepatic stem cell, hematopoietic stem cell, a neuronal stem cell, a muscle stem cell, an adipose stem cell, or in various combinations thereof.

This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

4. In Vitro and In Vivo Assays

Other aspects of the present invention involve a composition that provides increased transduction efficiency. Such compositions may be tested both in vitro, for transduction efficiency, and in vivo, for efficacy, insulin-induction, and the like. The various assays for use in determining such changes in function are routine and easily practiced by those of ordinary skill in the art.

In vitro assays involve the use of an isolated composition or cells transfected with the composition. A convenient way to monitor transduction efficiency is by use of a detectable label, and assess the quantity of the label in the cellular population. Alternatively, a functional read out may be preferred, for example, the ability to affect (i.e., promote growth of) a target cell or a host cell.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In vivo assays, such as an MDCK transcytosis system assay, also can be easily conducted (Mostov et al., 1986). In these systems, it again is generally preferred to label the test candidate constructs with a detectable marker and to follow the presence of the marker after administration to the animal, preferably via the route intended in the ultimate therapeutic treatment strategy. As part of this process, one would take samples of body fluids, and one would analyze the samples for the presence of the marker associated with the composition. “Detectable labels” are compounds or elements that can be detected due to their specific functional properties, or chemical characteristics, the use of which allows the peptide or protein to which they are attached to be detected, and further quantified if desired.

Alternatively, the construct is not labeled with a detectable marker an dan insulin level is measured/determined to follow the presence after administeration or delivery.

Many appropriate imaging agents are known in the art, as are methods for their attachment to proteins (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Protein sequences may also be reacted with an enzyme in the presence of a coupling agent such as glutaraldehyde or periodate. Conjugates with fluorescein markers are prepared in the presence of these coupling agents or by reaction with an isothiocyanate. Rhodamine markers can also be prepared.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III). In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵Iodine is often being preferred for use in certain embodiments, and technicium^{99m} and indium¹¹¹ are also often preferred due to their low energy and suitability for long range detection.

III. Protein/Polypeptide Conjugates

In certain embodiments, the composition of the present invention comprises a viral vector having a polynucleotide encoding islet cell differentiation transcription factor molecule conjugated to a targeting moiety. In preferred embodiments, the targeting moiety is a site-directing or targeting compound that improves the compositions ability to be site-specific in the host. The targeting moiety may be operatively linked or attached to the islet cell differentiation transcription factor molecule and/or the islet cell growth factor molecule. In addition to encompassing the delivery of purified compounds, the present invention further contemplates the delivery of nucleic acids that encode cognate compounds such as polypeptides. Therefore, according to the present invention, both purified compounds and nucleic acid sequences encoding that compound, e.g., a cytokine, may be delivered in conjunction with the composition of the present invention.

A. Enzymes

Various enzymes are of interest according to the present invention. Enzymes that could be conjugated to the islet cell differentiation transcription factor molecule, either directly or through a linking moiety, include cytosine deaminase, adenosine deaminase, hypoxanthine-guanine phosphoribosyltransferase, galactose-1-phosphate uridyltransferase, phenylalanine hydroxylase, glucose-6-phosphate dehydrogenase, HSV thymidine kinase, and human thymidine kinase and extracellular proteins such as collagenase and matrix metalloprotease, lysosomal glucosidase (Pompe's disease), muscle phosphorylase (McArdle's syndrome), glucocerebosidase (Gaucher's disease), α-L-iduronidase (Hurler syndrome), L-iduronate sulfatase (Hunter syndrome), sphingomyelinase (Niemann-Pick disease) and hexosaminidase (Tay-Sachs disease).

B. Drugs

According to the present invention, a drug may be operatively linked to a vector, or a linking moiety to deliver the drug to the liver and/or pancreas. It is contemplated that drugs such as antimetabolites (e.g., purine analogs, pyrimidine analogs, folinic acid analogs), enzyme inhibitors, metabolites, or antibiotics (e.g., mitomycin) are useful in the present invention. Small molecules are also included.

C. Antibody Regions

Regions from the various members of the immunoglobulin family are also encompassed by the present invention as suitable targeting moities. Both variable regions from specific antibodies are covered within the present invention, including complementarity determining regions (CDRs), as are antibody neutralizing regions, including those that bind effector molecules such as Fc regions. Antigen specific-encoding regions from antibodies, such as variable regions from IgGs, IgMs, or IgAs, can be employed with the islet cell differentiation transcription factor molecule complexed to the vector of the present invention in combination with an antibody neutralization region or with one of the therapeutic compounds described above.

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

Antibodies to a wide variety of molecules are contemplated, such as oncogenes, cytokines, growth factors, hormones, enzymes, transcription factors or receptors. Also contemplated are secreted antibodies targeted against serum, angiogenic factors (VEGF/VPF; βFGF; αFGF; and others), coagulation factors, and endothelial antigens necessary for angiogenesis (i.e., V3 integrin). Specifically contemplated are growth factors such as transforming growth factor, fibroblast growth factor, islet cell growth factors (i.e., BTC) and platelet derived growth factor (PDGF) and PDGF family members.

The present invention further embodies composition targeting specific pathogens through the use of antigen-specific sequences or targeting specific cell types, such as those expressing cell surface markers to identify the cell. Examples of such cell surface markers would include tumor-associated antigens or cell-type specific markers such as CD4 or CD8.

D. Regions Mediating Protein-Protein or Ligand-Receptor Interaction

The use of a region of a protein that mediates protein-protein interactions, including ligand-receptor interactions, also is contemplated by the present invention. This region could be used as an inhibitor or a competitor of a protein-protein interaction or as a specific targeting motif. Consequently, the invention covers using a polypeptide, such as a polypeptide having a binding domain, to recruit a protein region that mediates a protein-protein interaction to a somatic cell, including a pancreatic cell, a beta-cell, a liver cell, a progenitor cell, a stem cell, a pluripotent stem cell, a totipotent stem cell, a hepatocyte, a hematopoietic stem cell, a neuronal stem cell or a mixture thereof. Once the compositions of the present invention reach the cancer cell, more specific targeting of the composition is contemplated through the use of a region that mediates protein-protein interactions including ligand-receptor interactions.

Protein-protein interactions include interactions between and among proteins such as receptors and ligands; receptors and receptors; polymeric complexes; transcription factors; kinases and downstream targets; enzymes and substrates; etc. For example, a ligand binding domain mediates the protein:protein interaction between a ligand and its cognate receptor. Consequently, this domain could be used either to inhibit or compete with endogenous ligand binding or to target more specifically cell types that express a receptor that recognizes the ligand binding domain operatively attached to the islet cell differentiation transcription factor molecule or the islet cell growth factor molecule.

Examples of ligand binding domains include ligands such as VEGF/VPF; βFGF; αFGF; coagulation factors, and endothelial antigens necessary for angiogenesis (i.e., V3 integrin); growth factors such as transforming growth factor, fibroblast growth factor, colony stimulating factor, Kit ligand (KL), flk-2/flt-3, and platelet derived growth factor (PDGF) and PDGF family members; ligands that bind to cell surface receptors such as MHC molecules, among other.

The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, Nicolau et al. (1987) employed lactosyl-ceramide, a galactose-terminal asialganglioside, incorporated into liposomes and observed an increase in the uptake of the insulin gene by hepatocytes. Also, the human prostate-specific antigen (Watt et al., 1986) may be used as the receptor for mediated delivery to prostate tissue.

E. Growth Factors

In other embodiments of the present invention, growth factors or ligands will be encompassed by the second therapeutic agent or the targeting moiety. Examples include VEGF/VPF, FGF, TGFβ, ligands that bind to a TIE, tumor-associated fibronectin isoforms, scatter factor, hepatocyte growth factor, fibroblast growth factor, platelet factor (PF4), PDGF, KIT ligand (KL), colony stimulating factors (CSFs), LIF, and TIMP. In preferred embodiments, the growth factor is an islet cell growth factor, such as BTC polypeptide.

F. Hormones

Additional embodiments embrace the use of a hormone as a selective agent. For example, the following hormones or steroids can be implemented in the present invention: prednisone, progesterone, estrogen, androgen, gonadotropin, ACTH, CGH, or gastrointestinal hormones such as secretin.

G. Cell Cycle Regulators

Cell cycle regulators provide possible advantages as the second therapeutic agent, when combined with other genes. Such cell cycle regulators include p27, p16, p21, p57, p18, p73, p19, p15, E2F-1, E2F-2, E2F-3, p107, p130, and E2F-4. Other cell cycle regulators include anti-angiogenic proteins, such as soluble Flk1 (dominant negative soluble VEGF receptor), soluble Wnt receptors, soluble Tie2/Tek receptor, soluble hemopexin domain of matrix metalloprotease 2, and soluble receptors of other angiogenic cytokines (e.g., VEGFR1, VEGFR2/KDR, VEGFR3/Flt4, and neutropilin-1 and -2 coreceptors).

H. Linkers/Coupling Agents

If desired, dimers or multimers of the targeting moiety and islet cell differentiation transcription factor molecule and/or the islet cell growth factor molecule may be joined via a biologically-releasable bond, such as a selectively-cleavable linker or amino acid sequence. Alternatively, such constructs are employed in protein purification methods (see section titlted Proteinaceous Compositions). For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

It is also contemplated that a peptide containing multimers of the islet cell differentiation transcription factor molecule and/or the islet cell growth factor molecule may be comprised of heteromeric sequences, in which the binding sequences utilized are not identical to each other, or homomeric sequences, in which a binding domain sequence is repeated at least once. Amino acids such as selectively-cleavable linkers, synthetic linkers, or other amino acid sequences may be used to separate a binding domain from another binding domain. Alternatively, linker sequences may be employed both between at least once set of binding domains, as well as between a binding domain and a selective agent or compound. The term “binding domain” refers to at least one amino acid residue that is employed to link, conjugate, coordinate, or complex another compound or molecule, either directly (i.e., covalent bond) or indirectly (i.e., via a linking moiety).

Additionally, while numerous types of disulfide-bond containing linkers are known which can successfully be employed to conjugate the polypeptide having a therapeutic activity with the targeting moiety and/or linking moiety of the invention, certain linkers will generally be preferred over other linkers, based on differing pharmacologic characteristics and capabilities. For example, linkers that contain a disulfide bond that is sterically “hindered” are preferred, due to their greater stability in vivo, thus preventing release of the toxin moiety prior to binding at the site of action. Furthermore, while certain advantages in accordance with the invention will be realized through the use of any of a number of linking moieties, the inventors have found that the use of salicylhydroxamic acid will provide particular benefits. It is also contemplated that linkers are employed to conjugate the islet cell differentiation transcription factor gene with selective agents to, for example, aid in detection. Alternatively, biochemical cross-linkers are contemplated.

Generally, Cross-linking reagents are used to form molecular bridges that tie together functional groups of two different molecules, e.g., a stablizing and coagulating agent. To link two different proteins in a step-wise manner, hetero-bifunctional cross-linkers can be used that eliminate unwanted homopolymer formation. Non-limiting examples of hetero-bifunctional cross-linkers are listed in Table 3.

TABLE 3
HETERO-BIFUNCTIONAL CROSS-LINKERS

| | | | Spacer Arm |
|---|---|---|---|
| | | | Length\after cross- |
| Linker | Reactive Toward | Advantages and Applications | linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

It can therefore be seen that a targeted peptide composition will generally have, or be derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking. For a general overview of linking technology, one may wish to refer to Ghose & Blair (1987).

Once conjugated, the targeting peptide generally will be purified to separate the conjugate from unconjugated targeting agents or coagulants and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

IV. Nucleic Acids and Polynucleotides

In certain embodiments, the present invention is directed to administering or delivering a nucleic acid expressing an islet cell differentiation transcription factor polypeptide. In other embodiments, the present invention is directed to administering or delivering a nucleic acid expressing an islet cell growth factor polypeptide. The therapy involving administering in vivo and/or ex vivo of the compositions comprising a nucleic acid expressing one or more islet cell differentiation transcription factor polypeptides provide transgene expression of the polypeptide(s) in the liver of the mammal. Further, the administeration of cDNAs encoding the polypeptides individually or in various combinations increased insulin levels, increased the number of insulin-producing cells, and treated the disease in the host.

In certain embodiments, the nucleic acid sequence encodes a mammalian islet cell differentiation transcription factor nucleic acid sequence or is any sequence which is homologous to or has significant sequence similarity to said nucleic acid. As used herein, significant sequence similarity means similarity is greater than 25% and can occur in any region of another sequence.

In certain embodiments, the nucleic acid comprises a polynucleotide encoding a specific islet cell differentiation transcription factor protein and/or islet cell growth factor protein, such as a cDNA. If a cDNA is used in the composition, exemplary islet cell differentiation transcription factors include, but are not limited to a cDNA encoding for any of the following gene products: NeuroD, ngn3, Pax4, Pax6, Nkx2.2, Nkx6.1, Is1-1, or Pdx-1. Further, if a cDNA is used in the composition, exemplary islet cell growth factors include, but are not limited to a cDNA encoding for the BTC gene product.

In certain embodiments of the present invention, the islet cell differentiation transcription factor is provided as a nucleic acid expressing the islet cell differentiation transcription factors polypeptide. The nucleic acid expressing the polypeptide may be operably linked to a promoter. Non-limiting examples of promoters suitable for the present invention include any promoter operable in a eukaryotic cell, including, but not limited to CMV IE, dectin-1, dectin-2, human CD11c, F4/80, SM22, MHC class II promoter, BOS, and PEPCK, however, any other promoter that is useful to drive expression of the islet cell differentiation transcription factor gene or the islet cell growth factor gene of the present invention, such as those set forth herein, is believed to be applicable to the practice of the present invention. It is also contemplated that the promoter is provided by way of a vector, such as an expression vector, which are discussed in more detail below.

Specific promoters that may be useful in the present invention include but are not limited to the following: (1) the BOS promoter, which is the elongation factor 1-alpha promoter (Miszushima and Nagata, 1990); (2) the phosphoenolpyruvate carboxykinase (PEPCK) promoter (Beale et al., 1992);. (3) The CDK9 promoter (Liu and Rice, 2000); and (4) The beta actin promoter (Qin and Gunning, 1997). The PEPCK promoter is a liver-specific promoter that has been used previously by the inventors. The BOS promoter, a ubiquitous promoter that should be active even when cells are transdifferentiated into beta cells or any other cell type, has also been used previously by the inventors. The CDK9 promoter and the beta actin promoter also drive ubiquitous expression of transgenes.

Preferably, the nucleic acid of the present invention is administered by injection. Other embodiments include the administering of the nucleic acid by multiple injections. In certain embodiments, the injection is performed local, regional or distal to a diseased site. In preferred embodiments, the administering of nucleic acid is via systemic delivery, continuous infusion, intratumoral injection, intraperitoneal, or intravenous injection. Preferably the patient is a human. In other embodiments the patient is a diabetic patient.

In preferred specific embodiments, the nucleic acid encodes the amino acid sequence of SEQ ID NO:1, SEQ ID NO:45, SEQ ID NO:55, SEQ ID NO:68, SEQ ID NO:83, SEQ ID NO:85, SEQ ID NO:87, SEQ ID NO:89, and/or SEQ ID NO:91. In still further embodiments the nucleic acid encodes or encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, or 206 contiguous amino acids of SEQ ID NO:1, SEQ ID NO:45, SEQ ID NO:55, SEQ ID NO:68, SEQ ID NO:83, SEQ ID NO:85, SEQ ID NO:87, SEQ ID NO:89, and/or SEQ ID NO:91.

The present invention may involve nucleic acids, including an islet cell differentiation transcription factor-encoding nucleic acid, nucleic acid identical or complementary to all or part of the sequence of an islet cell differentiation transcription factor gene, as well as nucleic acid constructs and primers discussed herein.

These polynucleotides or nucleic acid molecules are isolatable and purifiable from mammalian cells. It is contemplated that an isolated and purified islet cell differentiation transcription factor nucleic acid molecule, either full-length or relatively truncated, that is a nucleic acid molecule related to the islet cell differentiation transcription factor gene product, may take the form of RNA or DNA. Similarly, the nucleic acid molecule related to the immunogenic molecule may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding islet cell differentiation transcription factor” refers to a nucleic acid segment that contains islet cell differentiation transcription factor coding sequences, such as those described above, yet is isolated away from, or purified and free of, total genomic DNA and proteins. When the present application refers to the function or activity of a islet cell differentiation transcription factor-encoding polynucleotide or nucleic acid, it is meant that the polynucleotide encodes a molecule that has the ability to increase an insulin level, to generate an insulin-producing cell, and to treat insulin-dependent diabetes in vitro (i.e., by way of administration ex vivo) or in vivo.

Further, a “polynucleotide encoding an islet cell growth factor” refers to a nucleic acid segment that contains an islet cell growth factor coding sequences, such as those discussed herein, yet is isolated away from, or purified and free of, total genomic DNA and proteins. When the present application refers to the function or activity of an islet cell growth factor-encoding an islet cell growth factor polypeptide or peptied, it is meant that the polynucleotide encodes a molecule that has the ability to induce and/or promote growth of an islet cell in vitro or in vivo.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 1989; Ausubel, 1996). There may be times when the full or partial genomic sequence is preferred. Alternatively, cDNAs may be advantageous because it represents coding regions of a polypeptide and eliminates introns and other regulatory regions.

It also is contemplated that a given islet cell differentiation transcription factor-encoding nucleic acid or islet cell differentiation transcription factor gene from a given cell may be represented by natural variants or strains that have slightly different nucleic acid sequences but, nonetheless, encode a islet cell differentiation transcription factor polypeptide; a human islet cell differentiation transcription factor polypeptide is a preferred embodiment. Consequently, the present invention also encompasses derivatives of islet cell differentiation transcription factor with minimal amino acid changes, but that possess the same activity.

The term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding islet cell differentiation transcription factor or another therapeutic polypeptide such as the islet cell growth factor may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to the respective SEQ ID NO:94 or SEQ ID NO:95 (NeuroD encoding sequences); to SEQ ID NO:98 or SEQ ID NO:98 (ngn3 encoding sequences); SEQ ID NO:100 or SEQ ID NO:101 (Pax4 encoding sequences); SEQ ID NO:102 SEQ ID NO:103 (Pax6 encoding sequences); SEQ ID NO:104 or SEQ ID NO:105 or SEQ ID NO:106 or SEQ ID NO:107 (Nkx2.2 encoding sequences); SEQ ID NO:108 or SEQ ID NO:109 or SEQ ID NO:110 or SEQ ID NO:111 (Nkx6.1 encoding sequences); SEQ ID NO:112 or SEQ ID NO:113 (Is1-1 encoding sequences); SEQ ID NO:114 or SEQ ID NO:115 (Pdx-1 encoding sequences); or SEQ ID NO:96 or SEQ ID NO:97 (BTC encoding sequences).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a NeuroD protein, polypeptide or peptide that includes within its amino acid sequence a contiguous amino acid sequence in accordance with, or essentially as set forth in, SEQ ID NO:1, corresponding to the NeuroD designated “human NeuroD” or “NeuroD polypeptide.” Similarly, where the invention concerns other isolated DNA segments and recombinant vectors incorporating DNA sequences that encode ngn3, Pax4, Pax6, Nkx2.2, Nkx6.1, Is1-1, Pdx-1 and BTC proteins, polypeptides or peptides, the same requirement for a contiguous amino acid sequence applies with respect to the respective sequences set forth above for each molecule, i.e. essentially as set forth in SEQ ID NO: 45, SEQ ID NO:83, SEQ ID NO:85, SEQ ID NO: 87, SEQ ID NO: 89, SEQ ID NO: 91, SEQ ID NO: 55, or SEQ ID NO: 68, respectively.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO: 1 will be sequences that are “essentially as set forth in SEQ ID NO:1” provided the biological activity of the protein is maintained. In particular embodiments, the biological activity of a NeuroD protein, polypeptide or peptide, or a biologically functional equivalent, comprises increasing an insulin level or generating an insulin-producing cell. The term “essentially as set forth in SEQ ID NO:94” is used in the same sense as described above and means that the nucleic acid sequence substantially corresponds to a portion of SEQ ID NO:94 and has relatively few codons that are not identical, or functionally equivalent, to the codons of SEQ ID NO:94. Again, DNA segments that encode proteins, polypeptide or peptides exhibiting NeuroD activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:45 will be sequences that are “essentially as set forth in SEQ ID NO:45” provided the biological activity of the protein is maintained. In particular embodiments, the biological activity of a ngn3 protein, polypeptide or peptide, or a biologically functional equivalent, comprises increasing an insulin level or generating an insulin-producing cell. The term “essentially as set forth in SEQ ID NO:98” is used in the same sense as described above and means that the nucleic acid sequence substantially corresponds to a portion of SEQ ID NO:98 and has relatively few codons that are not identical, or functionally equivalent, to the codons of SEQ ID NO:98. Again, DNA segments that encode proteins, polypeptide or peptides exhibiting ngn3 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:55 will be sequences that are “essentially as set forth in SEQ ID NO:55” provided the biological activity of the protein is maintained. In particular embodiments, the biological activity of a Pdx-1 protein, polypeptide or peptide, or a biologically functional equivalent, comprises increasing an insulin level or generating an insulin-producing cell. The term “essentially as set forth in SEQ ID NO:114” is used in the same sense as described above and means that the nucleic acid sequence substantially corresponds to a portion of SEQ ID NO:114 and has relatively few codons that are not identical, or functionally equivalent, to the codons of SEQ ID NO:114. Again, DNA segments that encode proteins, polypeptide or peptides exhibiting Pdx-1 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:68 will be sequences that are “essentially as set forth in SEQ ID NO:68” provided the biological activity of the protein is maintained. In particular embodiments, the biological activity of a BTC protein, polypeptide or peptide, or a biologically functional equivalent, comprises increasing an insulin level or generating an insulin-producing cell. The term “essentially as set forth in SEQ ID NO:96” is used in the same sense as described above and means that the nucleic acid sequence substantially corresponds to a portion of SEQ ID NO:96 and has relatively few codons that are not identical, or functionally equivalent, to the codons of SEQ ID NO:96. Again, DNA segments that encode proteins, polypeptide or peptides exhibiting BTC activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:83 will be sequences that are “essentially as set forth in SEQ ID NO:83” provided the biological activity of the protein is maintained. In particular embodiments, the biological activity of a Pax4 protein, polypeptide or peptide, or a biologically functional equivalent, comprises increasing an insulin level or generating an insulin-producing cell. The term “essentially as set forth in SEQ ID NO:100” is used in the same sense as described above and means that the nucleic acid sequence substantially corresponds to a portion of SEQ ID NO:100 and has relatively few codons that are not identical, or functionally equivalent, to the codons of SEQ ID NO:100. Again, DNA segments that encode proteins, polypeptide or peptides exhibiting Pax4 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:85 will be sequences that are “essentially as set forth in SEQ ID NO:85” provided the biological activity of the protein is maintained. In particular embodiments, the biological activity of a Pax6 protein, polypeptide or peptide, or a biologically functional equivalent, comprises increasing an insulin level or generating an insulin-producing cell. The term “essentially as set forth in SEQ ID NO:102” is used in the same sense as described above and means that the nucleic acid sequence substantially corresponds to a portion of SEQ ID NO:102 and has relatively few codons that are not identical, or functionally equivalent, to the codons of SEQ ID NO:102. Again, DNA segments that encode proteins, polypeptide or peptides exhibiting Pax6 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:87 will be sequences that are “essentially as set forth in SEQ ID NO:87” provided the biological activity of the protein is maintained. In particular embodiments, the biological activity of a Nkx2.2 protein, polypeptide or peptide, or a biologically functional equivalent, comprises increasing an insulin level or generating an insulin-producing cell. The terms “essentially as set forth in SEQ ID NO:104”, “essentially as set forth in SEQ ID NO:105” or “essentially as set forth in SEQ ID NO:106” is used in the same sense as described above and means that the nucleic acid sequences substantially corresponds to a portion of SEQ ID NO:104, SEQ ID NO:105, or SEQ ID NO:106, respectively, and has relatively few codons that are not identical, or functionally equivalent, to the codons of SEQ ID NO:104, SEQ ID NO:105, or SEQ ID NO:106, respectively. Again, DNA segments that encode proteins, polypeptide or peptides exhibiting Nkx2.2 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:89 will be sequences that are “essentially as set forth in SEQ ID NO:89” provided the biological activity of the protein is maintained. In particular embodiments, the biological activity of a Nkx6.1 protein, polypeptide or peptide, or a biologically functional equivalent, comprises increasing an insulin level or generating an insulin-producing cell. The terms “essentially as set forth in SEQ ID NO:108”, “essentially as set forth in SEQ ID NO:109” or “essentially as set forth in SEQ ID NO:110” is used in the same sense as described above and means that the nucleic acid sequences substantially corresponds to a portion of SEQ ID NO:108, SEQ ID NO:109, or SEQ ID NO:110, respectively, and has relatively few codons that are not identical, or functionally equivalent, to the codons of SEQ ID NO:108, SEQ ID NO:109, or SEQ ID NO:110, respectively. Again, DNA segments that encode proteins, polypeptide or peptides exhibiting Nkx6.1 activity will be most preferred.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO: 91 will be sequences that are “essentially as set forth in SEQ ID NO:91” provided the biological activity of the protein is maintained. In particular embodiments, the biological activity of a Is1-1 protein, polypeptide or peptide, or a biologically functional equivalent, comprises increasing an insulin level or generating an insulin-producing cell. The term “essentially as set forth in SEQ ID NO:114” is used in the same sense as described above and means that the nucleic acid sequence substantially corresponds to a portion of SEQ ID NO:114 and has relatively few codons that are not identical, or functionally equivalent, to the codons of SEQ ID NO:114. Again, DNA segments that encode proteins, polypeptide or peptides exhibiting Is1-1 activity will be most preferred.

A. Expression Elements and Vectors

In particular embodiments, the invention concerns isolated nucleic acid segments and recombinant vectors incorporating DNA sequences that encode islet cell differentiation transcription factor polypeptides or peptides and/or DNA sequences that encode islet cell growth factor polypeptides or peptides.

Vectors of the present invention are designed, primarily, to transform somatic cells with a therapeutic islet cell differentiation transcription factor gene under the control of regulated eukaryotic promoters (i.e., inducible, repressable, universal, tissue-specific). Also, the vectors may contain a selectable marker if, for no other reason, to facilitate their manipulation in vitro. However, selectable markers may play an important role in producing recombinant cells. Tables 3 and 4, below, list a variety of regulatory signals for use according to the present invention.

TABLE 3
Inducible Elements

| Element | Inducer | References |
|---|---|---|
| MT II | Phorbol Ester (TPA) | Palmiter et al., 1982; Haslinger and |
| | Heavy metals | Karin, 1985; Searle et al., 1985; |
| | | Stuart et al., 1985; Imagawa et al., |
| | | 1987; Karin et al., 1987; Angel et |
| | | al., 1987b; McNeall et al., 1989 |
| MMTV (mouse | Glucocorticoids | Huang et al., 1981; Lee et al., 1981; |
| mammary tumor virus) | | Majors and Varmus, 1983; |
| | | Yamamoto et al., 1983; Lee et al., |
| | | 1984; Ponta et al., 1985; Si.e.,i et al., |
| | | 1986 |
| β-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H- | Interferon | Blanar et al., 1989 |
| 2 κb | | |
| HSP70 | Ela, SV40 Large T Antigen | Taylor et al., 1989; Taylor and |
| | | Kingston, 1990a,b |
| Proliferin | Phorbol Ester-TPA | Mordacq and Linzer, 1989 |
| Tumor Necrosis Factor | PMA | Hensel et al., 1989 |
| Thyroid Stimulating | Thyroid Hormone | Chatterjee et al., 1989 |
| Hormone α Gene | | |

TABLE 4
Other Promoter/Enhancer Elements

| Promoter/Enhancer | References |
|---|---|
| Immunoglobulin Heavy Chain | Banerji et al., 1983; Gillies et al., 1983; Grosschedl and |
| | Baltimore, 1985; Atchinson and Perry, 1986, 1987; Imler |
| | et al., 1987; Neuberger et al., 1988; Kiledjian et al., 1988; |
| Immunoglobulin Light Chain | Queen and Baltimore, 1983; Picard and Schaffner, 1985 |
| T-Cell Receptor | Luria et al., 1987, Winoto and Baltimore, 1989; Redondo |
| | et al., 1990 |
| HLA DQ α and DQ β | Sullivan and Peterlin, 1987 |
| β-Interferon | Goodbourn et al., 1986; Fujita et al., 1987; Goodbourn |
| | and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; Johnson et |
| | al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, 1989 |
| □-Globin | Bodine and Ley, 1987; Perez-Stable and Constantini, |
| | 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell Adhesion Molecule | Hirsch et al., 1990 |
| (NCAM) | |
| a_{1-Antitrypain} | Latimer et al., 1990 |
| H2B (TH2B) Histone | Hwang et al., 1990 |
| Mouse or Type I Collagen | Rippe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al., 1987 Schaffner et al., 1988 |
| Polyoma | Swartzendruber and Lehman, 1975; Vasseur et al., 1980; |
| | Katinka et al., 1980, 1981; Tyndell et al., 1981; Dandolo et |
| | al., 1983; Hen et al., 1986; Si.e.,i et al., 1988; Campbell |
| | and Villarreal, 1988 |
| Retroviruses | Kriegler and Botchan, 1983; Kriegler et al., 1984a,b; Bosze |
| | et al., 1986; Miksicek et al., 1986; Celander and Haseltine, |
| | 1987; Thiesen et al., 1988; Celander et al., 1988; Chol et |
| | al., 1996; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika et |
| | al., 1987, Stephens and Hentschel, 1987 |
| Hepatitis B Virus | Bulla and Siddiqui, 1988; Jameel and Siddiqui, 1986; |
| | Shaul and Ben-Levy, 1987; Spandau and Lee, 1988 |
| Human Immunodeficiency Virus | Muesing et al., 1987; Hauber and Cullan, 1988; |
| | Jakobovits et al., 1988; Feng and Holland, 1988; Takebe |
| | et al., 1988; Berkhout et al., 1989; Laspia et al., 1989; |
| | Sharp and Marciniak, 1989; Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

The promoter used in the present invention is preferably operable in a cell in which insulin production will be effected by delivery of an islet cell differentiation transcription factor. Thus, the promoter should be useful in stem cells, liver cells, fat cells, pancreatic cells, and so forth.

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In some embodiments, the promoter for use in the present invention is the cytomegalovirus (CMV) promoter. This promoter is commercially available from Invitrogen in the vector pcDNAIII, which is preferred for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth honnone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In addition to the classical IRES elements referred to above, the are other internal ribosome entry sites that consist of short ligonucleotides of 9-nucleotide segments, that identified in the Gtx gene (Chappell et al. 2000, Proc Natl Acad Sci USA 97: 1536-1541)(Owens et al. 2001, Proc Natl Acad Sci USA 98: 1471-1476). Synthetic 9-nucleotide multimers of such sequence function efficiently as IRESes that have the advantage of being short and efficient functional moduels that can be easily used for the expression of multiple genes using a single promoter/enhancer.

In any event, it will be understood that promoters are DNA elements which when positioned functionally upstream of a gene leads to the expression of that gene. Most transgene constructs of the present invention are functionally positioned downstream of a promoter element.

In specific embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles. In more preferred embodiments, the viral vector is an adenoviral vector, a retroviral vector, a vaccinia viral vector, an adeno-associated viral vector, a polyoma viral vector, an alphaviral vector, a rhabdoviral vector, or a herpesviral vector. Most preferably, the viral vector is an adenoviral vector. In other specific embodiments, the nucleic acid is a non-viral vector. Non-limiting examples of suitable vectors are discussed below.

B. Viral Transformation

1. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

In preferred embodiments involving the first generation adenoviral vector, the viral vector is replication-deficient, and generation and propagation of the vector depend on a unique helper cell line, such as 293, which is transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current first generation adenovirus vector is under 7.5 kb, or about 15% of the total length of the vector. More than 80% of the adenovirus viral genome remains in the vector backbone.

In specifc embodiments, the present invention involves an adenoviral vector that has all endogenous viral protein genes deleted, designated “gutless adenoviral vector”, “gutted adenoviral vector”, “fully deleted adenoviral vector”, “high-capacity adenoviral vector”. This “gutless adenoviral vector” can be amplified (produced) by specialized cells, or it can be produced by a method that utilizes a helper adenovirus (helper virus). When the vector is called helper-dependent adenovirus (HDAd), but the vector used is identical to the “gutless vector”, the terms “gutless”, “gutted”, “fully deleted” and “helper-dependent or HD” adenoviral vector will be used interchangeably as they apply to the same adenoviral vector.

The HDAd differs from the first generation adenoviral vector in that all adenoviral protein genes are deleted from the vector backbone, which contains only the ITRs at the two ends and the packaging signal ψ sequence (Kochanek, 1999). HDAd can be amplified with or without the use of a helper-adenovirus (“helper virus”). As the rest of the adenovirus DNA is totally deleted, the maximum cloning capacity for HDAd is about 37 kb, which allows for the insertion of large transgenes together with different types of promoters.

In one embodiment, the HDAd is amplified with a helper virus, which is a first generation adenovirus with loxP sequences flanking the packaging signal, in a 293 cell line expressing Cre recombinase (Parks et al., 1996)). The “gutless adenoviral vector” can also be produced without a helper virus, or with helper virus and producer cell lines of a different design, including, but not confined to, the use of the FLP-frt system instead of the Cre-loxP system (Umana et al., 2001; Ng et al,. 2001).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line for producing adenoviral vector is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

3. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference. [0243] Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

4. Other Viral Vectors

Other viral vectors may be employed as constructs in the present invention (including, but not limited to those reviewed in Kay et al. (2002) Nature Medicine 7: 33-40). Vectors derived from viruses such as vaccinia virus (Ridgeway, 1988; Baichwal and Sugden, 1986; Coupar et al., 1988), lentivirus, and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acid encoding an islet cell differentiation transcription factor and/or an islet cell growth factor to be delivered is housed within an infective virus that has been engineered to express a specific binding ligand. The virus particle will thus bind specifically to the cognate receptors of the target cell and deliver the contents to the cell. Alternatively, the nucleic acid encoding the islet cell differentiation transcription factor polypeptide and/or an islet cell growth factor polypeptide to be delivered is housed within an infective virus that has been engineered to express an islet cell differentiation transcription factor and/or an islet cell growth factor product.

A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors. For example, targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

The above methods to provide a nucleic acid encoding a islet cell differentiation transcription factor polypeptide and/or an islet cell growth factor peptide or polypeptide are by way of a example and are considered to extend to methods of providing a nucleic acid encoding an islet cell differentiation transcription factor.

C. Lipid Mediated Transformation

In a further embodiment of the invention, the gene construct may be entrapped in a liposome or lipid formulation. Gene constructs that are contemplated in the present invention comprise islet cell differentiation transcription factor-encoding nucleic acid and/or islet cell growth factor-encoding nucleic acid. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular immune therapies.

In certain embodiments of the invention, the lipid vehicle may be complexed with a hemagglutinating virus (HVJ). This has been shown to facilitate fusion with the cell membrane and promote cell entry of lipid-encapsulated DNA (Kaneda et al., 1989). In other embodiments, the lipid vehicle may be complexed or employed in conjunction with nuclear non-histone chromosomal proteins (HMG-1) (Kato et al., 1991). In yet further embodiments, the lipid vehicle may be complexed or employed in conjunction with both HVJ and HMG-1.

V. Islet Cell Transplantation

Administering an islet cell differentiation factor polypeptide of the present invention generated large single cells in the liver tissue. These single cells are contemplated for the use of transplantation.

Applicants demonstrated treatment of a diabetic animal (i.e., mammal) using methods and compositions of the present invention to provide for the generation of pancreatic islet structures and detectable immunoreactive insulin, proinsulin, glucagon and pancreatic polypeptide levels therein in the liver of the treated mammal. Specifically, the determined density distribution of insulin-positive cells occurring as single cells and as islet-like clusters in the liver are summarized in Table 5. The cells that respond to the HDAd gene therapy in vivo appear not to be regular hepatocytes, but a subpopulation of stem cells or special cells that possess pluripotent potential in the liver.

Specifically, the singlet cells are liver cells that undergo differentiation, which indicates that treatment of a liver cell with compositions of the present invention in vitro or in vivo promote differentiation to an islet cell. This application is contemplated for any somatic cell, including a hepatic cell, a progenitor cell, i.e., a pluripotent stem cell, a totipotent stem cell, a neural stem cell, or a hematopoietic stem cell. Also advantageous is that the somatic (host) cell may be obtained from the patient in need of the transplant, which circumvents the need for immunosuppression of the patient prior to transplantation of the islet graft comprising the generated differentiated islet cells.

TABLE 5
Presence of Insulin-Producing Cells

| | | | Islet-like Cluster |
|---|---|---|---|
| | Single Insulin⁺ cells | Islet-like Cluster | (no. insulin⁺ |
| Treatment | (no. cells/mm²) | (no. clusters/mm²) | cells/cluster) |
| Non-diabetic | Not detected | Not detected | N/a |
| STZ | 0.08 + 0.03^{a} | Not detected | N/a |
| STZ + HDAd-P-Pdx-1 | 0.74 + 0.10* | 0.0010 + 0.0006 | 50.3 + 3.8 |
| HDAd-P-Pdx-1 | | | |
| STZ + HDAd-BTC | 0.12 + 0.01^{b} | Not detected | N/a |
| STZ + HDAd-ND | 5.10 + 1.32^{#} | 0.0670 + 0.0150^{#} | 30.6 + 8.6 |
| STZ + HDAd- | 5.23 + 1.05^{#} | 0.1100 + 0.0120^{##} | 49.2 + 6.1 |
| ND/BTC | | | |

Values are mean + SEM from three animals;
*p < 0.01 compared to a and b;
♯related to p < 0.05;
♯♯relates to p < 0.01 compared to the values in STZ + HDAD-P-Pdx-1

Therefore, it is contemplated that the methods of the present invention are employed using either embryonic stem cells or adult stem cells isolated from the liver or other sources including, but not limited to circulating stem cells, bone marrow and fat depots, to effect islet differentiation in vitro. The treatment of these stem cell populations using the compositions of the present invention generate large numbers of pancreatic islets that are subsequently employed in transplantation in diabetic patients, thereby alleviating the shortage of islet donors for the transplant procedure. Additionally, the immune response that often plagues islet grafts in which the islet cells are prepared or obtained from foreign tissue are overcome as the stem cells that serve as host for the treatment are obtained from the patient, i.e., the patient's liver. Thus, transplantation of the generated islets cells of the present invention mitigate the need for insulin injections.

Somatic cells useful according to the invention include, but not be limited to, pancreatic cells, e.g., non-islet pancreatic cells, pancreatic islet cells, islet cells of the beta-cell type, non-beta-cell islet cells, and pancreatic duct cells. These cell types may be isolated according to methods known in the art for ex vivo manipulation. See, e.g., Githens, 1988, Jour. Pediatr. Gastroenterol. Nutr. 7:486; Warnock et al., 1988, Transplantation 45:957; Griffin et al., 1986, Brit. Jour. Surg. 73:712; Kuhn et al., 1985, Biomed. Biochim. Acta 44:149; Bandisode, 1985, Biochem. Biophys. Res. Comm. 128:396; Gray et al., 1984, Diabetes 33:1055, all of which are hereby incorporated by reference. Also contemplated as target cells are cell mixtures comprising any of a hepatocyte, a mature liver cell, a progenitor cell including a stem cell, a pluripotent stem cell, a totipotent stem cell, a hematopoietic stem cell or a neuronal stem cell in various combinations thereof.

It is also contemplated that the generated islet cells are cryopreserved for storage. For use, the generated islet cell population preferably has the following characteristics; greater than 80% of cells are viable before cryopreservation; greater than 70% of cells are viable after thawing. Methods of transplantation via islet grafts is well known in the art and such methods techniques are readily available to the skilled artisan. It is contemplated that all generated islet cells used in transplantation processes and methods comply with current regulatory requirements.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Immunohistochemistry of HDAd-Pdx-1 Treated STZ Mice

The effect of HDAd-Pdx-1 on STZ mice was evaluated by measuring the fasting serum glucose level (A and C) and body weight (B and D). The fasting serum glucose levels and body weights in diabetic mice were taken before and after administration of HDAd-Pdx-1 gene therapy. The HDAd compositions comprised either a BOS promoter (B-Pdx-1, A and B) or a PEPCK promoter (P-Pdx-1, C and D) to control transgene expression. Two weeks after STZ treatment, the STZ mice were injected with saline as a control (STZ only, n=5), varied doses of HDAd-B-Pdx-1 (n=4 each) or empty vector (HDAd0, n=4). For P-Pdx-1 experiments, STZ only, n=12. The doses (particles/mouse) of HDAd-P-Pdx-1 used included 1×10¹¹ (n=9), 3×10¹¹ (n=7), 4×10¹¹ (n=5) and 5×10¹¹ (n=7). Data represent mean ±SEM. The “†” indicates that all mice in the respective group died.

### Example 2

### RT-PCR of Liver of HDAd-Pdx-1 Treated STZ Mice

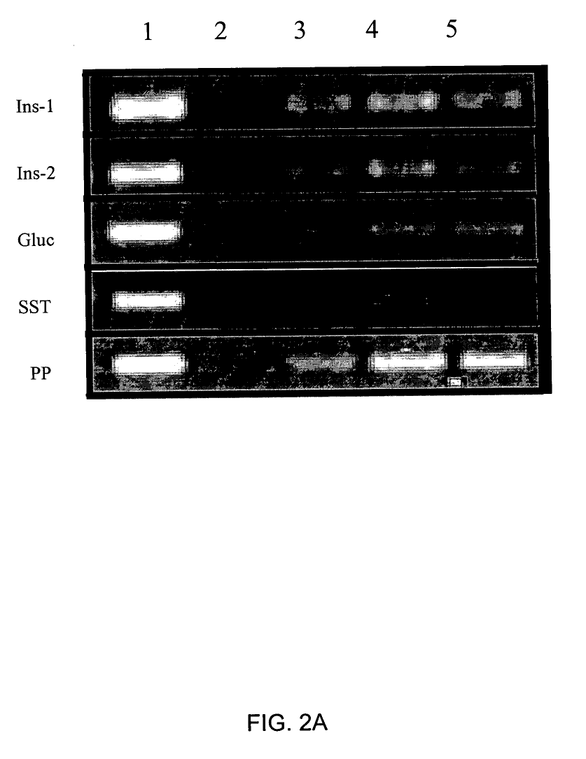

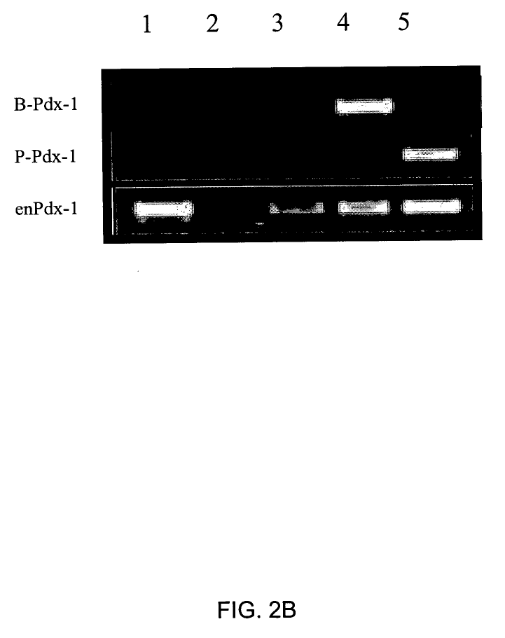

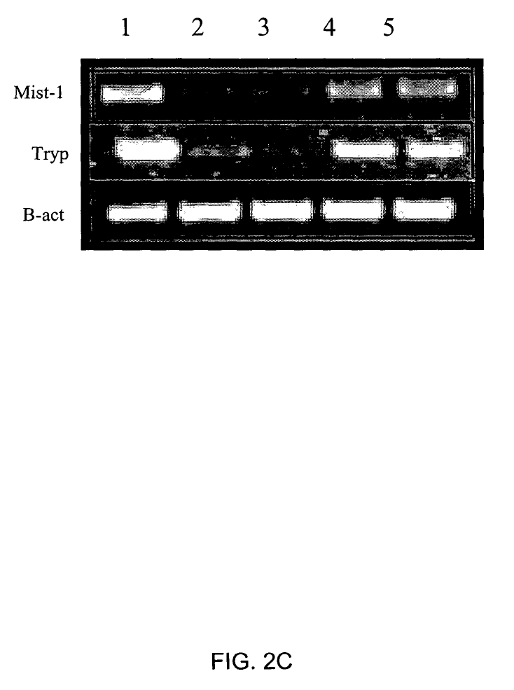

RT-PCR analysis of liver RNA was performed to evaluate the presence of islet-specific hormones and transcripts. FIGS. 2A-2C show the results of the analysis. The anaylsis included islet-specific hormones (FIG. 2A) mouse insulin 1 (Ins-1) and 2 (Ins-2), glucagon (Gluc) and somatostatin (SST). FIG. 2B shows the level of recombinant Pdx-1, wherein expression was controlled by either the BOS promoter (B-Pdx-10) or the PEPCK promoter (P-Pdx-1), and endogenous Pdx-1 (enPdx-1). FIG. 2C indicates the expression level of Mist1, trypsin (Tryp), and β-actin (B-act).

RNA was extracted 21-28 days from the liver after treatment with the designated HDAd composition. Lane 1, Normal mouse pancreas RNA; Lane 2, saline-treated non-diabetic liver; Lane 3, saline treated STZ mouse; Lane 4, STZ mouse liver treated with 3×10¹¹ particles/mouse of B-Pdx-1; Lane 5, STZ mouse liver treated with 3×10¹¹ particles/moouse of P-Pdx-1.

### Example 3

### Fluorescence Immunohistochemistry of HDAd-Pdx-1 Treated STZ Mice

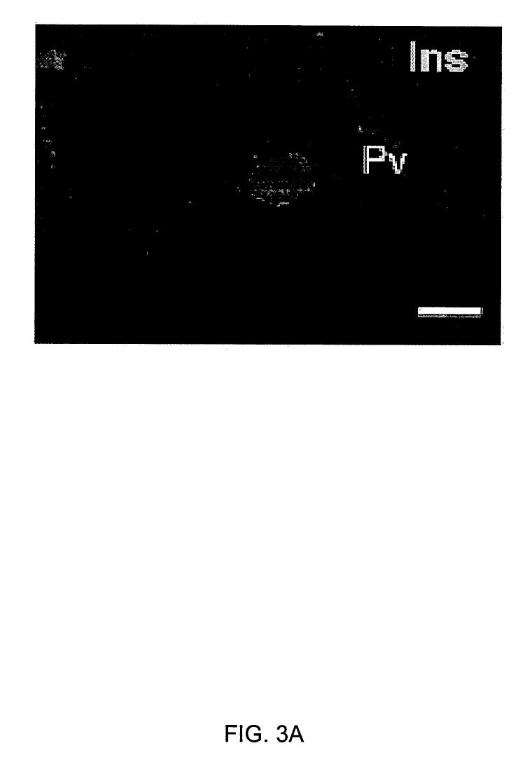

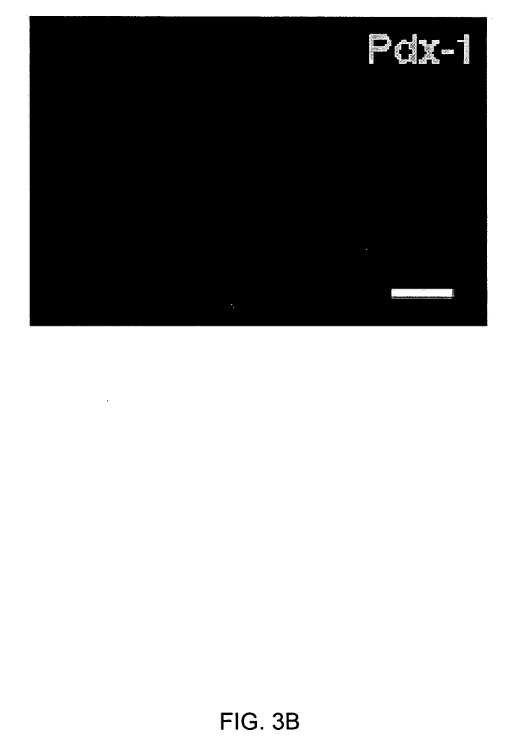

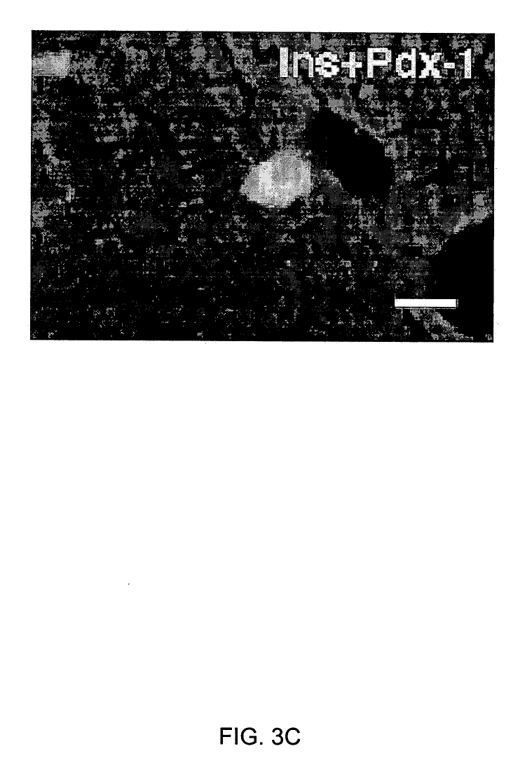

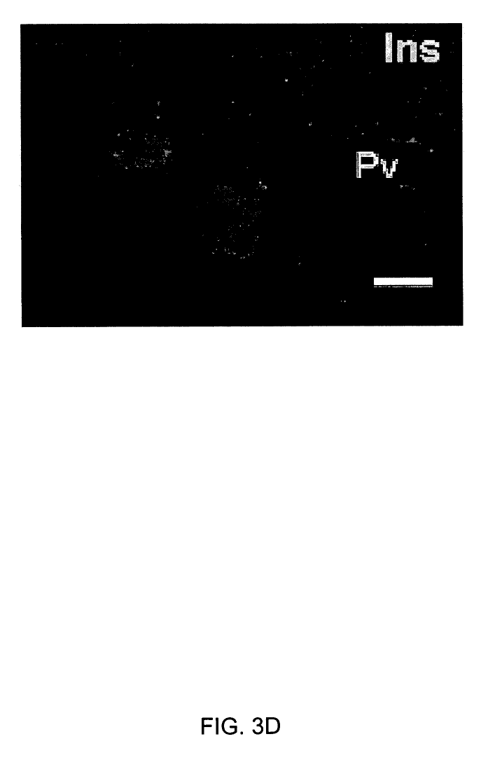

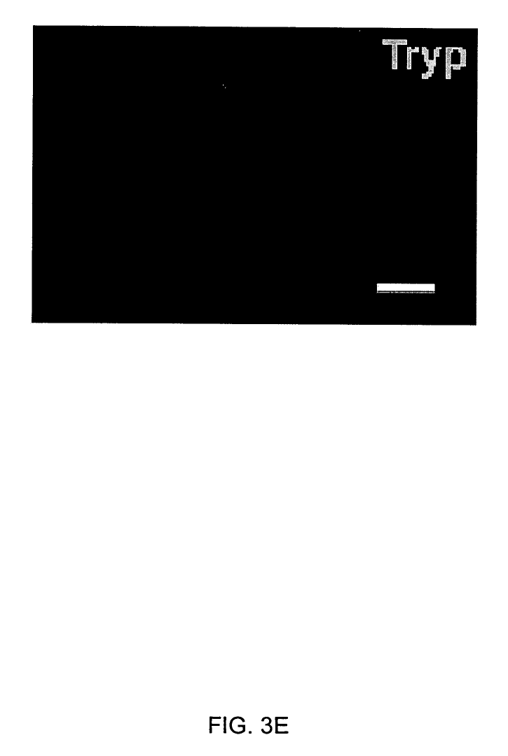

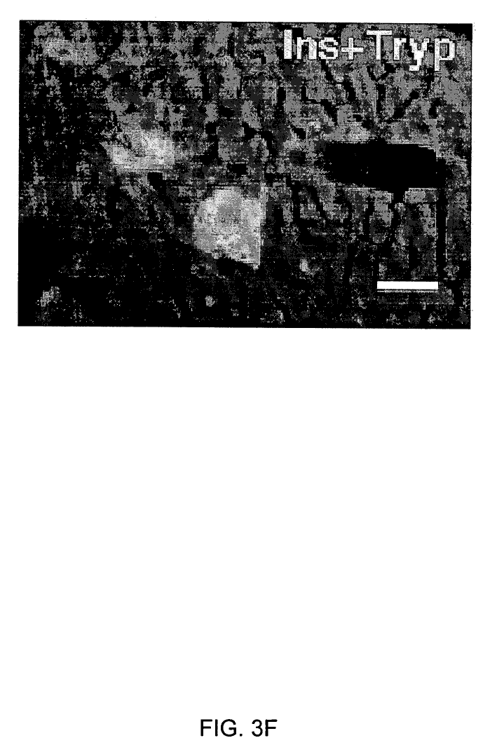

The fluorescence immunohistochemistry for insulin-producing cells in the liver of HDAd-Pdx-1 treated STZ mice (FIGS. 3D-3F) as compared to control (FIGS. 3A-3C) were observed. In the controls, a large insulin-positive cell (50 μm in diameter) is seen in the proximity of a portal vein (FIG. 3A). The large insulin-positive cell expresses immunoreactive PDX-1 in both nucleus and cytoplasm (FIG. 3B), and both PDX-1 and insulin are detected in the cytoplasm (FIG. 3C). In the liver sections of the treated mice, insulin-positive cells are scattered in the proximity of portal vein in the portal triad area (FIG. 3D). These insulin-positive cells also express immunoreactive trypsin (FIG. 3E), which co-localizes with insulin in the cytoplasm (FIG. 3F). Pv:portal vein. Bars=50 μm

### Example 4

### Liver Enzymes and Bilirubin Levels in HDAd-Pdx-1 Treated STZ Mice

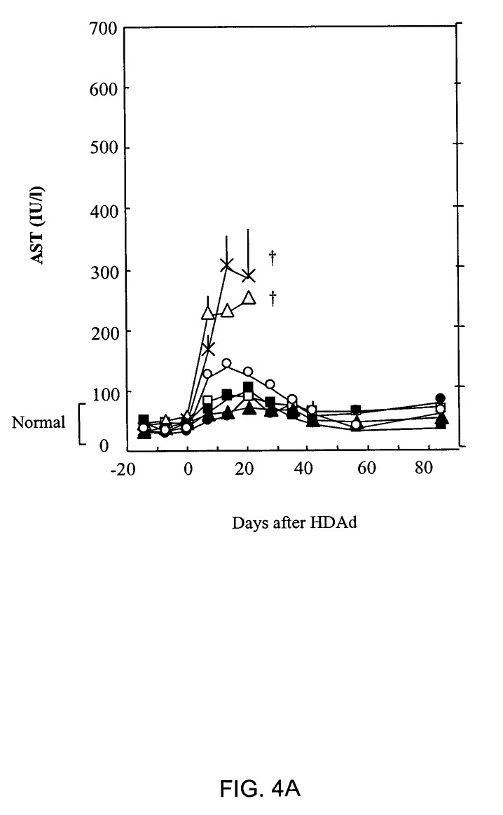

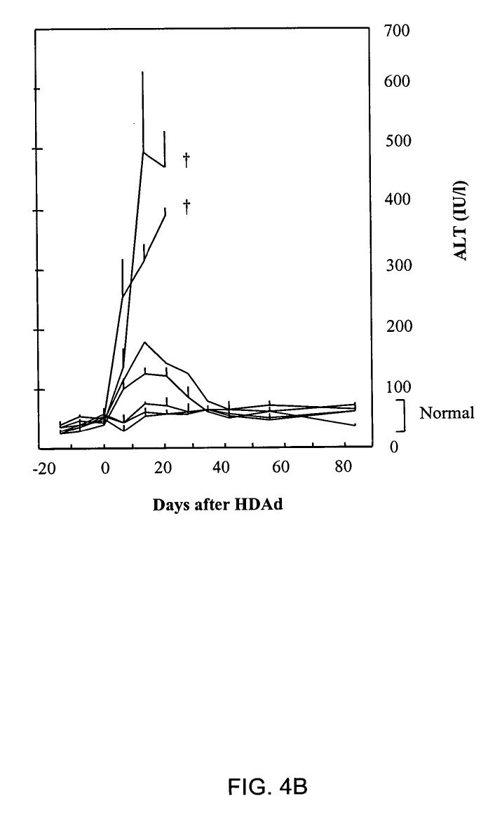

Post administration of the HDAd-Pdx-1 composition, the level of liver enzymes (FIGS. 4A and 4B) and bilirubin (FIG. 4C) determined in both the treated mice and the control mice. The serum level of asparate aminotransferase (AST) is shown in FIG. 4A and the serum level of alanine aminotransferase (ALT) is shown in FIG. 4B. The serum level of direct bilirubin was also measured (FIG. 4C). It was determined that Pdx-1 gene therapy treatment caused significant elevation of plasma asparate aminotransferase, alanine aminotransferase and bilirubin. These hepatotoxic complications were caused by Pdx-1, as treatment with an empty HDAd produced a negligible change in liver enzyme (not different from STZ alone) and no rise in bilirubin.

Values are mean ±SEM from 4 different animals. HDAd0 refers to an empty HDAd that contains no transgene. ND indicates NeuroD. BTC indicates betacellulin. †, indicates that all mice in the respective group died.

### Example 5

### HDAd Gene Therapy Effect on Serum Glucose, Insulin and Body Weight

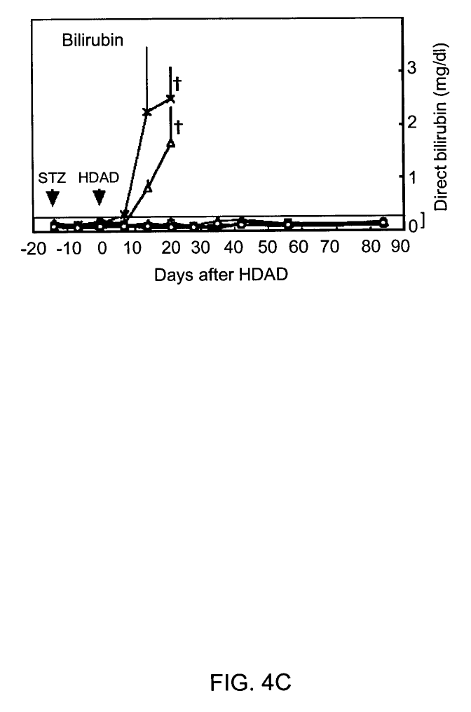

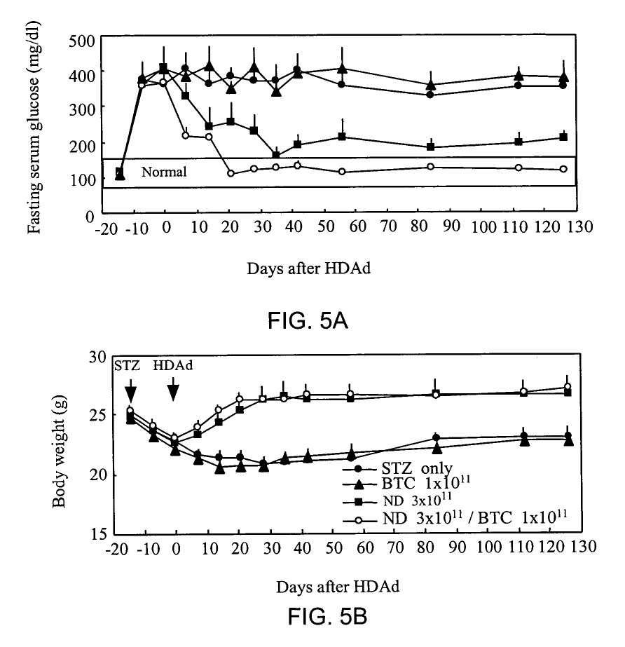

The effect of HDAd gene therapy on the fasting serum glucose level and body weight in treated STZ mice was analyzed as compared to control mice (untreated). FIGS. 5A and 5B show the the fasting serum glucose level (FIG. 5A) and body weight (FIG. 5B) of STZ mice treated with saline (STZ only, n=8), HDAd-BTC (BTC, 1×10¹¹, n=6), HDAd-NeuroD (ND 3×10¹¹, n=5), or HDAd-NeuroD(3×10¹¹) plus HDAd-BTC (1×10¹¹) (ND/BTC, n=5). Values are mean ±SEM. Serum glucose was normalized within 3 weeks and remained normal for >120 days. Importantly, regimens involving NeuroD or BTC did not cause any significant hepatotoxicity as indicated by the observed weight gain in mice after a single injection (FIG. 5B).

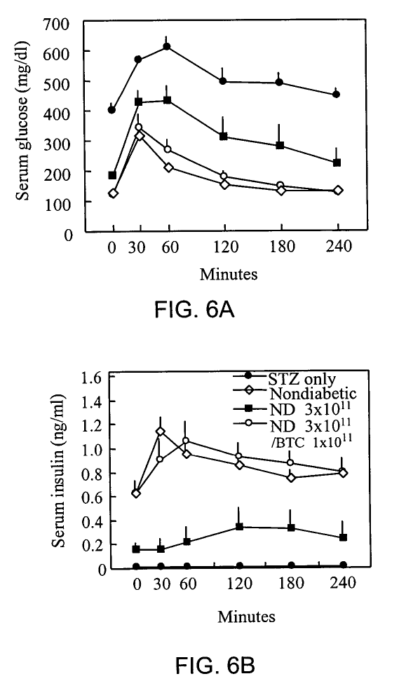

The effect of the HDAd gene therapy on serum glucose levels (FIG. 6A) and on serum insulin levels (FIG. 6B) were determined at 3 months post-treatment. The intraperitoneal glucose tolerance test (GTT performed at 1.5 g/kg body weight) revealed that the treated STZ mice had essentially undetectable insulin and persistently high serum glucose concentration. Animals treated with NeuroD displayed an improved but still diabetic curve. In contrast, STZ mice that received NeuroD/BTC combination therapy displayed normal glucose and insulin levels during a GTT (FIGS. 6A and 6B).

### Example 6

### mRNA Expression Levels in Liver of HDAd Treated STZ Mice

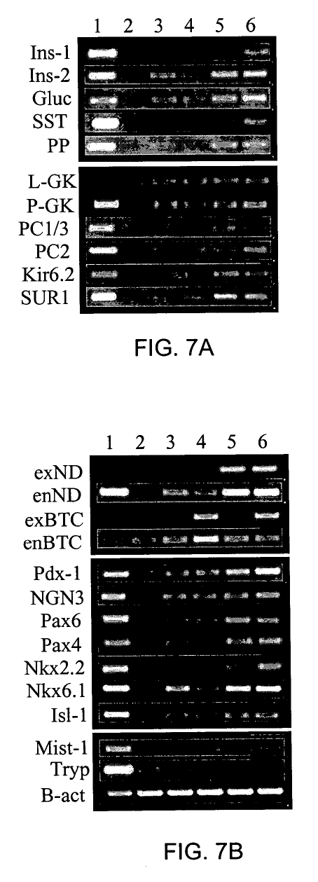

FIGS. 7A-7B shows results of RT-PCR analysis of liver RNA taken from STZ mice treated with HDAd gene therapy (lanes 4-6) as compared to control mice (lanes 1-3)

Liver transcripts: islet-specific hormones (FIG. 7A), various β cell-specific transcripts important for control of insulin production (FIG. 7A), vector-derived and endogenous NeuroD and BTC (FIG. 7B, exND and exBTC, vector-derived transcripts, enND and enBTC, endogenous transcripts), major transcription proteins involved in islet neogenesis (FIG. 7B) and exocrine-related transcripts (FIG. 7B). All determinations were made 4 months after saline or HDAd treatment. Lane 1, Normal mouse pancreas RNA; 2, saline-treated nondiabetic liver; 3, saline-treated STZ liver; 4, STZ liver treated with 1×10¹¹ particles/mouse of BTC. 5, STZ liver treated with 3×10¹ particles/mouse of ND. 6, STZ liver treated with 3×10¹¹ particles/mouse of ND plus 1×10¹¹ particles/mouse of BTC.

### Example 7

### Fluorescence Immunohistochemistry of Insulin-Producing Cells Generated From HDAd Treatment

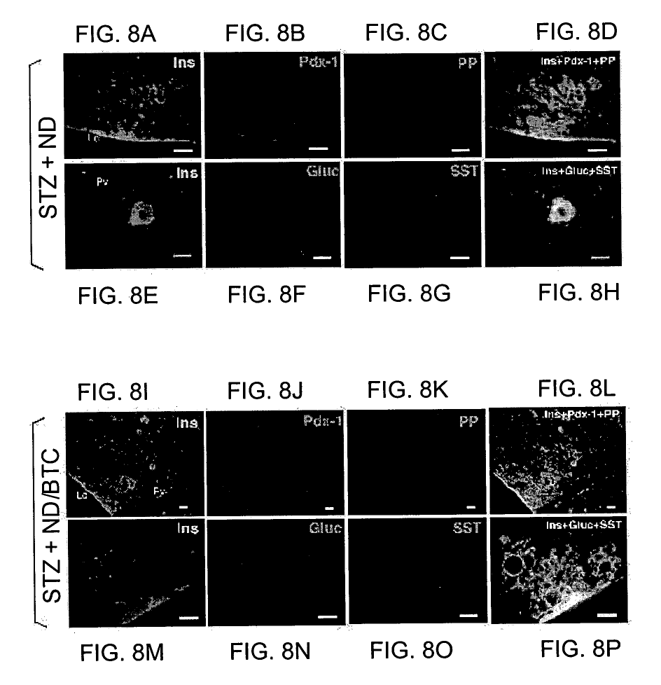

To characterize the generated insulin-producing cells observed after treatment with HDAd gene therapy in STZ mice, immunohistochemistry, electron microscopy and immuno-electron microscopy of liver sections at 4 months after a single treatment was performed. The compositions included in this evaluation HDAd comprising NeuroD and NeuroD plus BTC, at various doses: NeuroD-only (3×10¹¹ particles/mouse)-treated (FIGS. 8A-8H) and NeuroD (3×10¹¹ particles/mouse) plus BTC (1×10¹¹ particles/mouse)-treated mice (FIGS. 8I-8P). Massive aggregates of insulin-positive cells are seen immediately under the liver capsule (FIGS. 8A, 8I and 8M). The insulin-positive cells occur as single cells near a portal vein (FIG. 8E) or as clusters of insulin-positive cells in NeuroD/BTC-treated mice (FIGS. 8I and 8M) and in NeuroD-treated mice (FIG. 8A). These insulin-positive cells simultaneously express immunoreactive PDX-1 in both nuclei and cytoplasm (FIG. 8B and 8J), and complete overlap between PDX-1 and insulin staining in the cytoplasm (orange staining cells in FIGS. 8D and 8L) was observed.

A relatively small number of pancreatic polypeptide (PP)-positive cells are seen in the mid-region of the cluster (FIGS. 8C and 8K) and insulin-positive cells partially overlap with PP-positive cells (white staining cells in FIGS. 8D and 8L). Glucagon (FIG. 8N) and somatostatin (FIG. 80) are also stained in the clusters; the number of glucagon- or somatostatin-positive cells is smaller than that of insulin-positive cells (FIGS. 8M-8O). Glucagon and somatostatin staining completely overlaps each other; however, insulin staining only partially overlaps that of glucagon/somatostatin (white staining cells in FIG. 8P). The presence of cells that produce only insulin (green staining cells in FIG. 8P) is also observed. Single insulin-positive cells (FIG. 8E) that also stain with glucagon (FIG. 8F), and somatostatin (FIG. 8G) with complete overlap of hormone expression in the cytoplasm (white staining cell in FIG. 8H). Pv:portal vein. Lc:Liver capsule. Bars=50 μm.

### Example 8

### Electron Micrographs of Insulin-Producing Cells Generated from Treatment With HDAd Gene Therapy

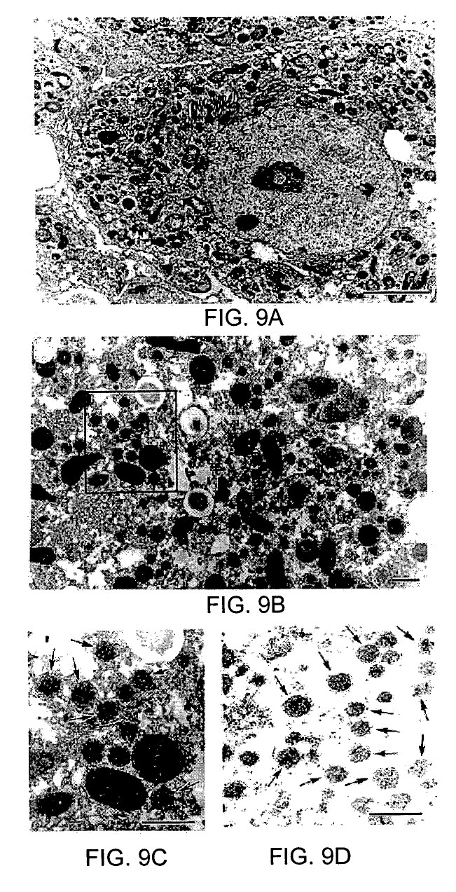

The insulin-producing cells generated from treatment with HDAd-NeuroD plus BTC gene therapy was evaluated by electron microscopy. The liver of STZ mice at 4 months post-treatment with HDAd-NeuroD (3×10¹¹ particles/mouse) plus -BTC (1×10¹¹ particles/mouse) demonstrated secretory granules densely packed in the cytoplasm (FIG. 9A). These granules are small (300-600 nm in diameter) and possess electron-dense cores. The endoplasmic reticulum was observed to be well-developed. FIGS. 9B and 9C are views of the secretory granules at a higher magnification. Crystalline formation of granular core is prominent, which is characteristic for β cells in rodents (arrows in FIG. 9C). FIG. 9D illustrates the cells post-embedding immunogold reaction for insulin. Immunogold particles are concentrated over the secretory granules (arrows) and a few are scattered over the cytoplasm. Bars=5 μm (a) and 1 μm (b-c).

### Example 9

### Detection of Insulin-Producing Cells in HDAd-Pdx-1 Treated STZ Mice

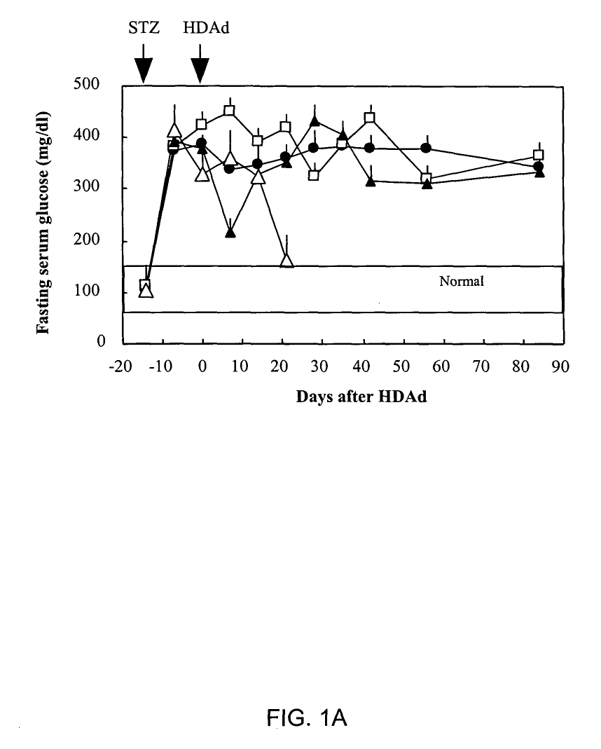

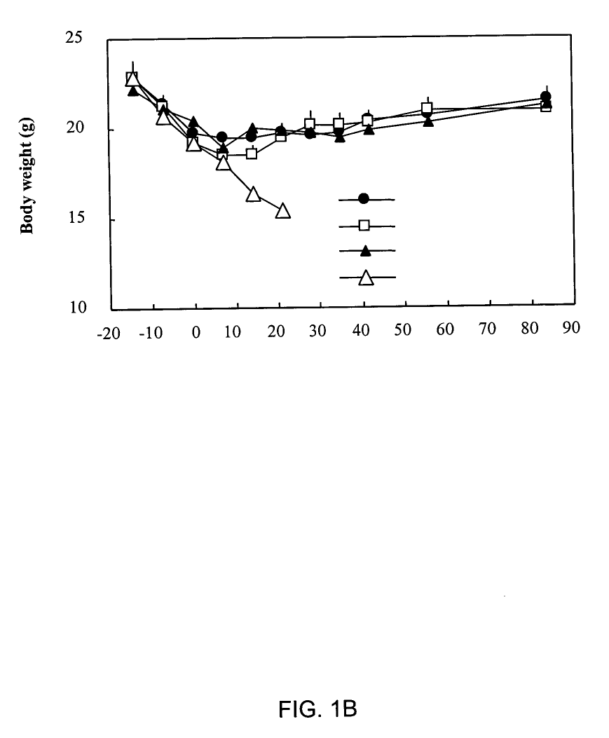

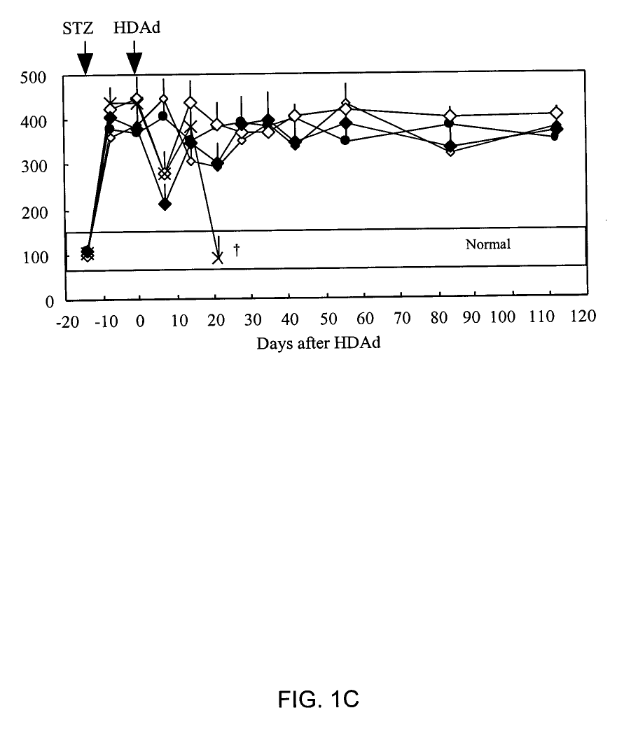

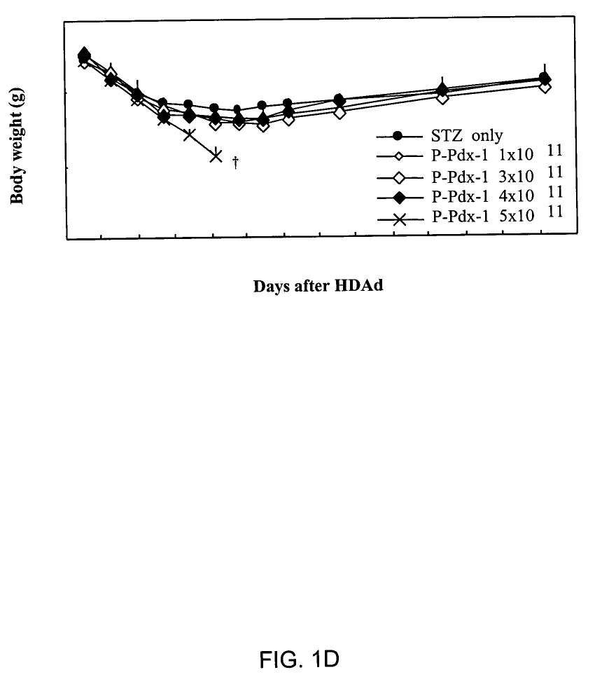

The immunohistochemistry analysis of the liver of HDAd-Pdx-1-treated STZ mice revealed the presence of large insulin-positive cells located mainly in the proximity of portal veins (FIGS. 1C and 1D). Using reverse-transcription nested PCR (RT-PCR), insulin-1 and insulin-2 transcripts, as well as transcripts for glucagon, somatostatin (SST) and PP, in HDAd-Pdx-1-treated animals (FIG. 1B) were also detected. Unexpectedly, insulin transcripts together with traces of transcripts of other islet hormones (FIG. 2A) were detected in STZ mice, but not in wild-type nondiabetic mice. Searching through multiple liver sections, rare insulin-positive cells were detected in all STZ mice examined. These cells were much larger (50 μm in diameter) than normal hepatocytes and occurred mainly in the portal triad region (FIG. 3A). They also expressed PDX-1 (FIGS. 3A-C), which was of endogenous origin, as these animals did not receive Pdx-1 therapy.

The appearance of insulin-producing cells in diabetic mouse liver is consistent with the recent report that adult rodent oval “stem” cells trans-differentiate into insulin-producing cells when they are exposed to a high-glucose environment (Yang et al., 2002). Despite an extensive search, detection of insulin transcripts by RT-PCR or of insulin-positive cells by immunohistochemistry in the liver of wild-type nondiabetic mice was not observed. Thus, the high blood glucose in STZ mice appeared to have induced β-cell differentiation in hepatic stem cells in situ. Insulin-positive cells occurred more frequently in STZ mice treated with HDAd-Pdx-1 as compared with those untreated control STZ mice, indicating that Pdx-1 gene therapy facilitated the trans-differentiation.

### Example 10

### Pdx-1 Administration Induced Death

Pdx-1 treatment caused significant elevation of plasma asparate aminotransferase, alanine aminotransferase (FIG. 4A) and bilirubin (FIG. 4B). These hepatotoxic complications were determined to be caused by Pdx-1 because treatment with an empty HDAd produced a negligible change in liver enzyme (i.e., not different from STZ alone) and no increase in bilirubin. Thus, the data suggested that Pdx-1 induced the appearance of pancreatic exocrine function. This is consistent with previous reports that exocrine and endocrine cells are derived from Pdx-1-expressing progenitors throughout embryogenesis (Gu et al., 2002). Consistent with this suggestion, the expression of Mist1, a pancreatic acinar cell-specific transcription factor downstream of Pdx-1 (Pin et al., 2001) was stimulated by Pdx-1 (FIG. 2C). Furthermore, Pdx-1 also stimulated trypsin mRNA accumulation in the liver (FIG. 2C) and the appearance of immunoreactive trypsin, which colocalized to insulin-positive cells (FIGS. 3D-F). Thus, Pdx-1-induced insulin expression was coupled to the expression of trypsin, and possibly other exocrine enzymes, in the same target cells, causing the latter to self-destruct as expression increased. This Pdx-1-induced suicide specifically destroys insulin-producing cells, accounting for the self-limiting nature of the hypoglycemic effect, and ultimately fatal outcome, of Pdx-1 gene therapy.

Pdx-1 gene therapy has been described previously, and no heptatoxicity or lethality was mentioned (Ferber et al., 2000). However, a FGAd was used to deliver Pdx-1 to the liver and produced hypoglycemia in STZ mice, and the experiment was terminated within 8 days of treatment as gene expression induced by FGAds is transient. The HDAd-Pdx-1 composition of the present invention delivered to the liver of STZ mice produced hypoglycemia that lasted only about a week (FIGS. 1A-1D). Further, replacement of a universal promoter with a liver-specific promoter did not change the outcome. Increasing the dose of Pdx-1 caused a greater glucose lowering, but had no effect on the duration of the hypoglycemic response. At the highest doses tested (3 and 5×10¹¹ particles/mouse), all treated mammals lost weight and, unexpectedly, died within 4 weeks (FIG. 1A). This demonstrated significant toxicity was not observed when administering similar doses of an empty HDAd or administering HDAds to deliver the other transgenes. Thus, the lethal outcome of the gene transfer was specific for Pdx-1. Based on these data, it is contemplated that the use of an FGAd, which are themselves highly hepatotoxic (O'Neal et al., 1998; O'Neal et al., 1998), for gene delivery creates a background that masks the hepatotoxicity of Pdx-1.

### Example 11

### HDAd-NeuroD, HDAd-BTC Compositions and Administration

As high-dose Pdx-1 therapy was detrimental and low-dose Pdx-1 was ineffective in the treatment of diabetes (FIGS. 1A, 4A and 4B), the use of NeuroD (also called Beta2) was explored, a basic helix-loop-helix transcription factor downstream of Pdx-1. NeuroD is required for proper morphogenesis of pancreatic islets and mice lacking NeuroD die of severe diabetic ketoacidosis shortly after birth (Naya et al., 1997). Increasing doses of an HDAd delivering NeuroD were administered to STZ mice. A relatively high dose (3×10¹¹ particles/mouse) of the vector produced a sustained, but incomplete, reversal of the hyperglycemia (FIGS. 5A and 5B). The co-administration of a β-cell stimulating hormone, betacellulin (BTC), to the regimen was evaluated. Although the HDAd-mediated delivery of BTC alone (1×10¹¹ particles/mouse) had no effect on the serum glucose of STZ mice, the combination of HDAd-mediated co-delivery of NeuroD (3×10¹¹ particles/mouse) and BTC (1×10¹¹ particles/mouse) completely reversed the diabetes. Serum glucose was normalized within 3 weeks and remained normal for >120 days (FIGS. 5A and 5B). Importantly, regimens involving NeuroD or BTC did not cause any significant hepatotoxicity; mice started to gain weight after a single injection (FIG. 5B).

At 3 months after treatment, an intraperitoneal glucose tolerance test (GTT) revealed that STZ mice had essentially undetectable insulin and persistently high serum glucose concentration. Animals treated with NeuroD displayed an improved but still diabetic curve. In contrast, STZ mice that received NeuroD/BTC combination therapy displayed normal glucose and insulin levels during a GTT (FIGS. 6A and 6B).

### Example 12

### HDAd-NeuroD, HDAd-BTC Compositions and Administration

The STZ induction of diabetes in mice led to low-level expression of insulin and other islet hormones (FIGS. 7A and 7B). HDAd-BTC alone had little or even a negative effect on the level of different islet-specific transcripts. HDAd-NeuroD gene therapy stimulated the level of insulin-2, glucagon, somatostatin (SST) and PP transcripts. HDAd-NeuroD/BTC, in contrast, stimulated the expression of all islet hormones, including insulin-1, insulin-2, glucagon, somatostatin and PP. Many of the β cell-specific transcripts, with exception of the proinsulin-processing enzyme PC1/3, were detectable in liver RNA of STZ mice. HDAd-NeuroD, with or without BTC, stimulated the expression of many of these transcripts, including those for the two proinsulin-processing enzymes PC1/3 and PC2, and the ATP-sensitive K⁺ channel subunits, Kir6.2 and sulfonylurea receptor (SUR1) (FIGS. 7A and 7B). Glucokinase (GK) is normally expressed in both liver and β cells, but the mRNAs in the two tissues are controlled by different promoters utilizing distinct transcription initiation sites. Pancreatic-type GK (P-GK) mRNA expression was undetectable in wild-type nondiabetic mouse liver, but was stimulated with the induction of β-cell formation in the liver by STZ treatment. It was further increased in HDAd-NeuroD-treated mice (FIGS. 7A and 7B), indicating a switch in promoter utilization from a liver to a β cell-specific mode.

### Example 13

### Effect of HDAd Treatment on Network of Transcriptional Factors

The transcriptional network of factors involved in pancreatic islet neogenesis were examined. Individual administration of HDAd-NeuroD (exND) and HDAd-BTC (exBTC) led to the expression of the respective vector-derived transcripts (FIGS. 7A and 7B). STZ-induced diabetes per se stimulated the appearance of NeuroD. Using endogenous NeuroD mRNA-specific primers identified that HDAd-NeuroD treatment stimulated endogenous NeuroD expression. Furthermore, NeuroD treatment also stimulated Pdx-1 expression over and above that seen in STZ mice. This stimulated expression resulting from HDAd-NeuroD treatment was also observed for the major factors involved in islet development, including neurogenin3, Pax6, Pax4, Nkx2.2, Nkx6.1 and Is1-1(FIGS. 7A and 7B). Therefore, HDAd-NeuroD treatment, with or without BTC, stimulated the expression of transcription factors that are upstream as well as downstream of NeuroD.

Although Pdx-1 expression was stimulated by HDAd-NeuroD, with or without BTC, there was no evidence of significant hepatotoxicity in these animals which continued to gain weight (FIGS. 5A and 5B) during HDAd-NeuroD treatment. Further, a mild transient increase in liver enzymes was observed, which was much less than that in HDAd-Pdx-1-treated mice (FIGS. 4A and 4B). Serum bilirubin remained normal (FIG. 4C), and no change was detected in the basal Mist1 expression (FIG. 7B). However, a trace amount of the trypsin transcript was detectable by RT-PCR in the liver of mice, which was increased substantially by HDAd-Pdx-1 (FIG. 2C), but unchanged by any of the regimens involving NeuroD or BTC (FIG. 3C). Finally, by immunohistochemistry no trypsin was detected in the liver of HDAd-NeuroD or HDAd-NeuroD/BTC-treated animals.

### Example 14

### Cellular Morphology in Liver Post-HDAd Treatment

HDAd-BTC treatment produced no change in liver morphology. HDAd-NeuroD induced the appearance of insulin-positive cells that also stained positive for PDX-1, PP, glucagon and somatostatin (FIG. 8A-8H). These cells occurred either as single large cells located usually near a portal vein (FIG. 8E-8H), or as clusters mostly under the liver capsule (FIG. 8A-8D), and the cells had a tendency to express multiple hormones and only a small minority expressed a single type of hormone.

After HDAd-NeuroD/BTC therapy, islet clusters that were located usually close to the liver capsule were detected (FIGS. 8I-8P). These cells expressed Pdx-1, as well as insulin, glucagon, somatostatin and PP, in various combinations. Further, a small population of these cells expressed solely insulin (FIG. 8P, green staining cells) or solely one of the other islet hormones. Immediately underneath the capsule were commonly sheets of cells that expressed the four islet hormones measured (FIGS. 8L and 8P, white-staining region). Further, cells occurring singly close to portal veins were found and simultaneously stained positive for the four islet hormones.

The number and distribution of insulin-producing cells in STZ mice following treatment with the different regimens was determined and is summarized in Table 5. Insulin-producing cells were not detected in the liver of nondiabetic mice. Such cells were rare, but detectable after a careful search, in all STZ mice that were examined. The insulin-producing cells occurred as single cells close to portal veins, and rarely, if at all, as clusters. HDAd-Pdx-1 therapy stimulated the appearance of these single cells about ten-fold. Only extremely rarely were islet clusters detected under the liver capsule in HDAd-Pdx-1-treated animals. HDAd-BTC treatment did not affect the frequency of insulin-producing cells. HDAd-NeuroD, on the other hand, increased the frequency of single insulin-producing cells >50 fold above that in STZ mice. It also led to the appearance of islet clusters under the liver capsule. Compared with HDAd-NeuroD alone, HDAd-NeuroD/BTC combination therapy did not affect the frequency of single insulin-positive cells, but further stimulated the occurrence of islet clusters by 60-70%. Interestingly, while there was a large difference in the number of islets formed in response to the different regimens, the number of insulin-positive cells remained relatively constant at 30-50 cells/islet cluster in the different treatment groups (Table 5).

### Example 15

### Characterization of Insulin-Producing Cells from Large Islet Clusters

Electron microscopic observation of insulin-producing cells from large islet clusters in the liver after HDAd-NeuroD/BTC therapy was performed. These cells possessed secretory granules densely packed in the cytoplasm (FIG. 9A) and contained no glycogen granules, suggesting that the cells had lost the properties of liver cells but acquired those of endocrine cells. The secretory granules were small (300-600 nm in diameter) and possessed electron-dense cores. Crystalline formation was prominent in the cores of the granules, a feature that is characteristic of secretory granules of normal pancreatic β cells in rodents¹⁸ (FIGS. 9B and 9C).

By immuno-electron microscopy, the presence of insulin-specific immunogold particles distributed mainly over the cores of secretory granules (FIG. 9D) was detected, indicating that they were insulin-containing granules characteristic of those found in beta-cells of rodents.

### Example 16

### Beta-Cell Transplantation

Because insulin production is highly complex and secretion is controlled mostly at the posttranscriptional and posttranslational levels, insulin transgenes that are regulated at the transcriptional level cannot respond to the minute-to-minute changes in blood glucose during meals and exercise. Insulin gene transduction also fails to induce beta-cell-specific molecules, such as beta-cell-specific glucokinase, SUR1 and Kir6.2, and proinsulin-processing enzymes, that are required for the fine-tuning of insulin production. Furthermore, insulin produced as a result of insulin gene transfer is released from the target cell via the constitutive pathway, a process that is unregulated and unresponsive to the individual's second-to-second metabolic needs (Halban et al., 2001). In contrast, ultrastructural and immuno-electron microscopic analysis of beta-cells induced by treatment of a diabetes with compositions of the present invention (i.e., HDAd-NeuroD/BTC ) reveals the presence of authentic appearing insulin granules and suggests that the insulin is secreted by regulated exocytosis as occurs in normal pancreatic beta-cells.

Another consequence of the treatment strategy of the present invention is the appearance of pancreatic islet-like structures that produce all the major islet hormones. This is contemplated to be important to the overall control of insulin production, as beta-cells normally do not work in isolation, and no significant hypoglycemia was observed in STZ mice that received even higher doses of compositions of the present invention. Futhermore, the data indicates that the presence of glucagon- and somatostatin-producing cells in the treated mammals equipped them with normal counter-regulatory mechanisms that made them extremely sensitive to the ever changing metabolic demands of the body.

In the diabetic subjects treated with HDAd-Pdx-1 or HDAd-NeuroD, many of the insulin-producing cells occurred in the portal triad region, a location that suggests that they could have come from hepatic “stem” cells lining the canals of Hering (Theise et al., 1999). Possibly they represent the same hepatic “stem” cells that were shown to differentiate into beta-cells when they were exposed to high glucose in vitro (Yang et al., 2002), but the beta-cells generated by methods and compositions of the present invention occurred in singlets, and often in complete isolation from other beta-cells. With HDAd-NeuroD treatment, and more particularly HDAd-NeuroD/BTC, treatment, the islet clusters detected occurred mostly under the liver capsule, produced all the major islet hormones and were present in dense patches. It is contemplated that the detected cellular structures represent islet neogenesis resulting from the trans-differentiation of normal hepatocytes. Histological analysis showed that the normal hepatic lobular architecture was preserved, making it unlikely that massive cellular proliferation had taken place. Thus, the compositions are suitable for use in methods to generate insulin-producing cells, and further in islet grafts for transplantation.

### Example 17

### Methods of Administration

Male C57/BL6 mice were purchased from The Jackson Laboratory, and maintained on a regular chow diet. Diabetes was induced by intravenous injection of streptozotocin (STZ, 100 mg/kg BW) at 8-10 weeks of age. Serum glucose was determined after a 10-h fast, before, and 7 and 14 days after, STZ treatment; mice with glucose levels of 250-600 mg/dl were selected for experiments. HDAds were injected systemically via the tail vein 14 days after STZ injection and all fasting serum glucose measurements were done after a 10-h fast.

### Example 18

### Methods of Constructing Recombinant Adenoviral Vector

Mouse Pdx-1-, NeuroD- and betacellulin-cDNAs were cloned into a KS vector by reverse transcription (RT)-PCR of total cellular RNA prepared from the pancreas of C57BL/6 mice. The fully sequenced cDNAs were subcloned into pLPBL1 shuttle plasmid (Oka et al., 2001) with a BOS promoter from elongation factor-lac (Mizushima et al., 1990) and human GH polyadenylation signal. The BOS promoter is a universal promoter for eukaryotes, which means that it is operable in a eukaryotic cell. In certain specific embodiments, phosphoenolpyruvate carboxykinase (PEPCK) promoter (Beale et al., 1992) and bovine beta-globin polyadenylation signal was employed and to denote the PEPCK promoter, the vector name was preceded by a “P”. The PEPCK promoter is a liver-specific promoter, which means that it is operable in a hepatic cell. The pΔ28 plasmid was used as backbone for all HDAds (Oka et al., 2001), which were amplified by the method of Parks et al., 1996.

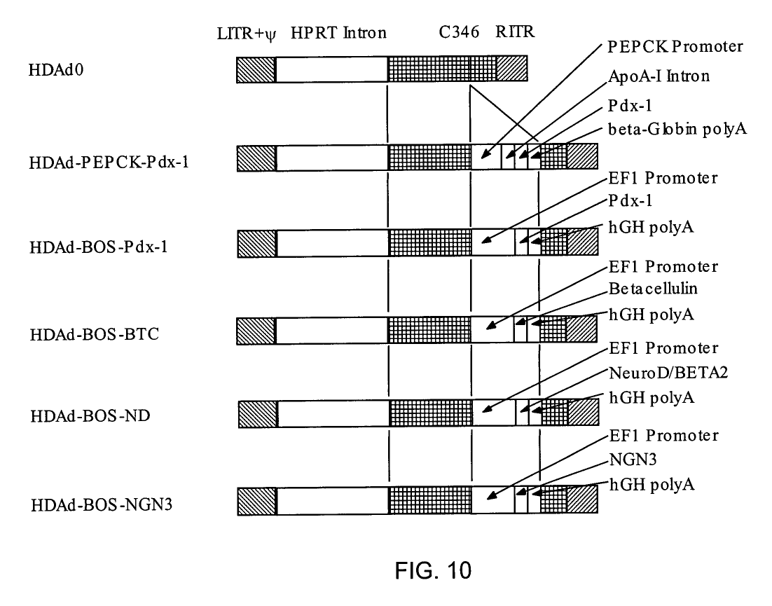

Examples of helper-dependent adenoviral vectors useful in the present invention are illustrated in FIG. 10.

### Example 19

### Methods of Intraperitoneal Glucose Test (GTT)

Three months after treatment with HDAd gene therapy, mice were fasted, and glucose solution (1.5 g/kg body weight) was injected into the peritoneal space. Blood was removed before, and 30, 60, 120, 180, and 240 minutes after glucose load. Serum was separated by centrifugation, and frozen at −20° C. until glucose and insulin determinations.

### Example 20

### Analysis of mRNA Expression

Animals were sacrificed on day 25-28 (Pdx-1 study) or 110-112 (NeuroD study), and their livers were removed and homogenized in acid guanidinium-phenol-chloroform (TRIzol, GIBCO BRL Co.) and total RNA was extracted and stored at −80° C. until analysis. Specific transcript level was quantitated by RT-PCR. For the detection of exogenous vector-derived Pdx-1, NeuroD and BTC transcripts, the forward primers were set within the coding sequence and the reverse primers were set within the vector-specific 3′-untranslated region, and 35 cycles of amplification was performed. To measure endogenous Pdx-1, NeuroD and BTC transcripts, the reverse primers corresponded to a region in the natural 3′-untranslated region of the transcript and 40 cycles of amplification was performed. Detecting islet-specific transcripts, including islet hormones and other, cell-specific molecules or trypsin, involved the use of forward and reverse primers within the coding sequence, and 35, 35, and 40 cycles of PCR, respectively. All PCR products were first confirmed by direct sequencing and are listed in Table 6 below.

TABLE 6
RT-PCR primers for the detection of specific transcripts.

| | Forward | |
|---|---|---|
| Name of primer | Reverse | SEQ ID NO |
| BOS-Pdx-1 | 5′-GTACTGCCTACACCCGGGCG-3′ | SEQ ID NO:116 |
| | 5′-AGGCAGCCTGCACCTGAGGAG-3′ | SEQ ID NO:117 |
| PEPCK-Pdx-1 | 5′-GTACTGCCTACACCCGGGCG-3′ | SEQ ID NO:118 |
| | 5′-TGCAACTTCCCAAGGCAGGA-3′ | SEQ ID NO:119 |
| BOS-NeuroD | 5′-GAAAGCCCCCTAACTGACTGC-3′ | SEQ ID NO:120 |
| | 5′-AGGCAGCCTGCACCTGAGGAG-3′ | SEQ ID NO:121 |
| BOS- | 5′-ATGGACCCAACAGCCCCGGG-3′ | SEQ ID NO:122 |
| BTC | | |
| | 5′-AGGCAGCCTGCACCTGAGGAG-3′ | SEQ ID NO:123 |
| Insulin-1 | 5′-ATGGCCCTGTTGGTGCACTTCC-3′ | SEQ ID NO:124 |
| | 5′-TTAGTTGCAGTAGTTCTCCAGCTGG-3′ | SEQ ID NO:125 |
| Insulin-2 | 5′-ATGGCCCTGTGGATGCGCTT-3′ | SEQ ID NO:126 |
| | 5′-CTAGTTGCAGTAGTTCTCCAGCTGG-3′ | SEQ ID NO:127 |
| Glucagon | 5′-ATGAAGACCATTTACTTTGTGGCTG-3′ | SEQ ID NO:128 |
| | 5′-CGGCCTTTCACCAGCCACGC-3′ | SEQ ID NO:129 |
| Somatostatin | 5′-ATGCTGTCCTGCCGTCTCCA-3′ | SEQ ID NO:130 |
| | 5′-CTAACAGGATGTGAATGTCTTCCAGAAGAA-3′ | SEQ ID NO:131 |
| PP | 5′-ATGGCCGTCGCATACTGCTG-3′ | SEQ ID NO:132 |
| | 5′-TCGCTCCAGGGCGCAGAGC-3′ | SEQ ID NO:133 |
| BTC | 5′-ATGGACCCAACAGCCCCGGG-3′ | SEQ ID NO:134 |
| | 5′-AGCTGTTTTCCTGAGACATGTCCTG-3′ | SEQ ID NO:135 |
| P-trypsin | 5′-GGAGCTGCTGTTGCTTTCCCTG-3′ | SEQ ID NO:136 |
| | 5′-AGCAGGTCTGGGTTGTTCACAC-3′ | SEQ ID NO:137 |
| P-GK | 5′-GGCCCAGAGAGTTACCTGTTGCC-3′ | SEQ ID NO:138 |
| | 5′-GCGCCATCCTGGCTCTGTCATCCAGC-3′ | SEQ ID NO:135 |
| L-GK | 5′-GCGGAAGTCCTTGGCTGC-3′ | SEQ ID NO:140 |
| | 5′-ACCAGAATCAACAACTGGGC-3′ | SEQ ID NO:141 |
| PC1/3 | 5′-ATGGAGCAAAGAGGTTGGACTCTGC-3′ | SEQ ID NO:142 |
| | 5′-GATTCCACATTGGATCATTGAAGCT-3′ | SEQ ID NO:143 |
| PC2 | 5′-ATGGAGGGCGGTTGTGGATC-3′ | SEQ ID NO:144 |
| | 5′-CAGGTACCATTGCTTTGTAAAGAGA-3′ | SEQ ID NO:145 |
| Kir6.2 | 5′-ATGCTGTCCCGAAAGGCCAT-3′ | SEQ ID NO:146 |
| | 5′-GGTCACCTGGACCTCGATGGAGAAA-3′ | SEQ ID NO:147 |
| SUR1 | 5′-ATGCCCTTGGCCTTCTGCG-3′ | SEQ ID NO:148 |
| | 5′-GTGATGAAGGCCAAGGTCCAGTAGAT-3′ | SEQ ID NO:149 |
| Pdx-1 | 5′-ATGAACAGTGAGGAGCAGTACTACGCG-3′ | SEQ ID NO:150 |
| | 5′-GGAGCCCAGGTTGTCTAAAT-3′ | SEQ ID NO:151 |
| NGN3 | 5′-GCGCAACAGGCCCAAGAGCG-3′ | SEQ ID NO:152 |
| | 5′-TCACAAGAAGTCTGAGAACA-3′ | SEQ ID NO:153 |
| NeuroD | 5′-GAAAGCCCCCTAACTGACTGC-3′ | SEQ ID NO:154 |
| | 5′-GCACTTTGCAGCAATCTTAGCAAAA-3′ | SEQ ID NO:155 |
| Pax4 | 5′-GGCCGTGAGCAAGATCCTAGGACG-3′ | SEQ ID NO:156 |
| | 5′-GCGCGAGAGGTGGCAGCAGCCAGC-3′ | SEQ ID NO:157 |
| Pax6 | 5′-ATGCAGAACAGTCACAGCGG-3′ | SEQ ID NO:158 |
| | 5′-TCGCTAGCCAGGTTGCGAAG-3′ | SEQ ID NO:159 |
| Nkx2.2 | 5′-ATGTCGCTGACCAACACAAA-3′ | SEQ ID NO:160 |
| | 5′-TCCTTGTCATTGTCCGGTGA-3′ | SEQ ID NO:161 |
| Nkx6.1 | 5′-GGCCGAGTGATGCAGAGTCCGCCG-3′ | SEQ ID NO:162 |
| | 5′-GCGCCCTCCTCATTCTCCGAAGTC-3′ | SEQ ID NO:163 |
| Isl-1 | 5′-ATGGGAGACATGGGCGATCC-3′ | SEQ ID NO:164 |
| | 5′-CGTGGTCTGCACGGCAGAAA-3′ | SEQ ID NO:165 |
| Mist1 | 5′-ATGAAGACCAAAAACCGGCCC-3′ | SEQ ID NO:166 |
| | 5′-CTAGCTCCCCTCTCTGAAGCTG-3′ | SEQ ID NO:167 |
| B-actin | 5′-ATGGATGACGATATCGCTGCGC-3′ | SEQ ID NO:168 |
| | 5′-TCTGTCAGGTCCCGGCCA-3′ | SEQ ID NO:169 |

### Example 21

### Fluorescence Immunohistochemistry

The liver was fixed and 20 μm-thick sections were processed for fluorescence overlap staining as follows. For double staining of insulin/PDX-1 or insulin/trypsin, the sections were incubated for 3 days with a mixture of antibodies against insulin (guinea-pig polyclonal, Linco Research Inc) and against PDX-1 (rabbit polyclonal, Watada et al., 1996). Alternatively, a mixture of antibodies against insulin and against trypsin (rabbit polyclonal, Biogenesis Ltd) was used. Each mixture was diluted 1:5000 in PBST at 4° C. To triple staining insulin/PDX-1/pancreatic polypeptide or insulin/glucagon/somatostatin, sections were incubated with a mixture of antibodies against insulin, PDX-1 and pancreatic polypeptide (mouse monoclonal, Yanaihara Ins.) or a mixture of insulin, glucagon (rabbit polyclonal, Biogenesis Ltd.) and somatostatin (mouse monoclonal, Fujimiya et al., 1992). The triple staining procedure was performed similarly to the double staining procedure described above.

The sections were then incubated for 2 hours with a mixture of Alexa Fluor® 488 -labeled anti-guinea-pig IgG (Molecular probes, Inc) and Alexa Fluor® 568 -labeled anti-rabbit IgG (Molecular probes, Inc) for double staining, or a mixture of Alexa Fluor® 488 -labeled anti-guinea-pig IgG, Alexa Fluor® 568 -labeled anti-rabbit IgG and Cy5-labeled anti-mouse IgG (Chemicon) for triple staining, diluted 1:1000 in PBST at room temperature. Sections were mounted on glass slides, dried, coverslipped with Histofine® (Nichirei Corp.) and observed under a fluorescence microscopy (Olympus, BX61). The image was transferred to Meta Morph image analyzing system (Nippon Roper Co).

### Example 22

### Identification of Insulin-Positive Cells by Immuno-Electron Microscopy

To identify insulin-positive cells by immuno-electron microscope, the liver was fixed and processed for insulin immunohistochemistry by ABC and DAB-nickel methods. Stained sections were osmificated and dehydrated with a graded series of ethanol and propylene oxide, and embedded in epoxy resin. Ultra-thin sections were cut in an ultramicrotome. They were stained with 2% uranyl acetate followed by Reynolds′ solution, and observed under electron microscopy (H-7100, Hitachi CO., Tokyo, Japan).

For post-embedding immunogold reactions, 20 μm liver sections were dehydrated with a graded series of ethanol and embedded in LR Gold resin (Ted Pella, Inc) as described by Fujimiya et al., 1997. The embedded specimens were polymerized for 4 h at −20° C. in an Ultraviolet Cryo Chamber (Pelco) and ultra-thin sections were cut and picked up on nickel grids. The grids were incubated with antibody against insulin (guinea-pig polyclonal antibody) diluted 1:40 in a reaction buffer for 2 h at RT and then incubated with immunogold-conjugated anti-guinea-pig IgG (10 nm gold) diluted 1:40 for 1.5 h at RT. The sections were stained with 2% uranyl acetate followed by Reynold's solution to prepare for observation by electron microscopy.

### Example 23

### Materials and Methods

Commercial kits were used for the determination of serum levels of glucose, aspartate aminotransferase (AST), alanine aminotransferase (ALT), direct bilirubin (Sigma), and insulin (Crystal Chem Inc.).

Statistical analysis was performed by ANOVA with SIGMASTAT (SPSS), and significance was assigned at p<0.05. All results were expressed as mean ±SEM.

### Example 24

### Treatment of Diabetic Mice with ngn3

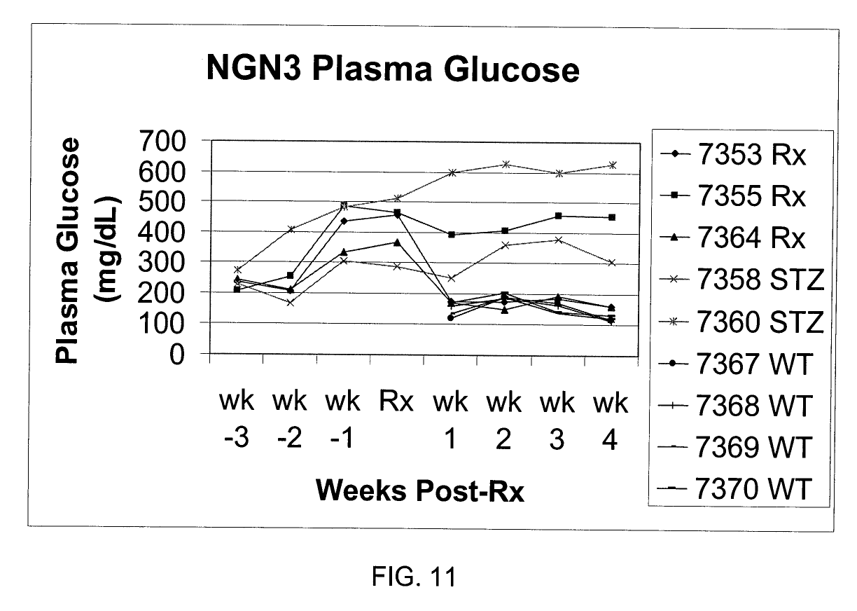

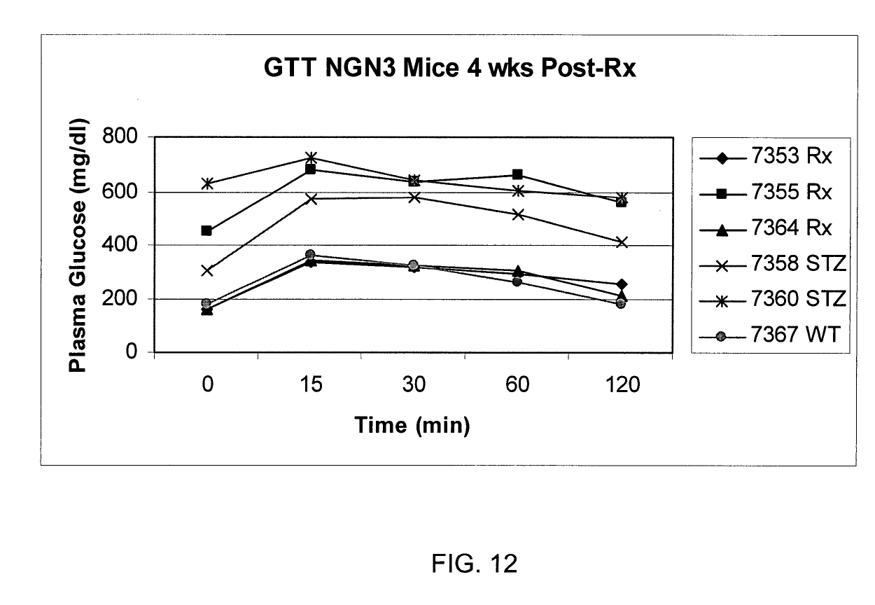

Diabetic mice were treated with neurogenin3 (ngn3), which is immediately upstream of NeuroD. FIGS. 11 and 12 provide data of blood glucose and glucose tolerance test (GTT), respectively, for three types of mice: (1) untreated nondiabetic C57BL/6 mice (labeled WT); (2) streptozoptocin-induced diabetic mice treated with HDAd expressing mouse ngn3 (labeled Rx), dose: 3×10¹¹+betabcellulin 1×10¹¹ (dose and construct similar to the other Examples); and (3) streptozotocin-induced diabetic mice, no treatment, labeled STZ.

As shown in FIG. 11, two of the three ngn3-treated mice developed normal blood glucose after 1 week, in comparison to the 3 weeks required for NeuroD-treated mice. Response of diabetic mice to NeuroD was good, and the improved response with ngn3 indicates that ngn3, a transcription factor upstream of NeuroD, is more effective than NeuroD.

FIG. 12 provides the GTT for these mice. The three curves in the bottom are identical, indicating that the ngn3-treated mice were indistinguishable from nondiabetic mice. This perfect GTT contrasts with that seen with the NeuroD-treated mice. The curve for NeuroD was statistically the same as normal, but actually the peak glucose for NeuroD was at 60 minutes, whereas the peak glucose for both ngn3-treated and nondiabetic mice are at 15 minutes. This is an indication of a normal “first phase” insulin response, which is defective in type 1 and type 2 diabetes. Therefore, ngn3 restores totally normal blood glucose dynamics, with restoration of a “first phase” insulin response as well as second phase response. NeuroD, in contrast, restored the second phase response in STZ mice, but a defective first phase response remains.

### Example 25

### Treatment of NOD Mice

The NOD mice are a special strain of mice that spontaneously develop autoimmune diabetes. These mice are widely used as a mouse model of type 1 diabetes in humans. These mice develop spontaneous diabetes with insulin deficiency caused by autoimmune destruction of the pancreatic islets.

The present inventors followed 8 NOD mice for months and treated them several weeks after they spontaneously developed diabetes as manifested by appearance of hyperglycemia (high blood glucose). They were treated with the same regimen as in Kojima et al. (2003) Nature Medicine 9: 596-603, (i.e., NeuroD plus betacellulin delivered by HDAd as described elsewhere herein). Over the next 1-5 weeks, 4 of the 8 diabetic NOD mice exhibited a normalization of their blood glucose. The blood glucose in these mice has remained normal for over two months.

These animals can be analyzed in a similar manner to what is described with the streptozotocin-induced diabetic mice discussed elsewhere herein. The most important conclusion is that about 50% of the NOD mice with autoimmune diabetes responded to the treatment. Without desiring to be bound by theory, there are at least two possible explanations for the response: (1) the newly formed islets in the liver are protected against the autoimmunity-mediated destruction; and/or (2) the treatment itself might alter the autoimmunity in these mice so their newly formed islets are free from this problem. Regardless, the success with these studies indicates that patients with type 1 autoimmune diabetes will also respond to the same treatment by forming new islets in the liver with alleviation of their diabetes.

### Example 26

### Treatment of Diabetic Higher Mammals

In specific embodiments, a higher mammal such as a monkey or human is treated using the present invention. In some embodiments, an exemplary animal model comprising a nonhuman primate is used, such as baboon or rhesus monkey. Both types of monkey can be rendered diabetic by streptozotocin treatment. In specific embodiments, a dosage of about 150 mg/Kg streptozotocin is administered to generate diabetes in the monkey, although other dosages may be utilized. After the streptozotocin treatment, the majority of the monkeys develop diabetes over the next 1-3 days. After they develop diabetes, i.e., high blood glucose, they are treated with appropriate fluid therapy and insulin regimens to keep their blood glucose below 200 mg/dl. The normal blood glucose in these animals are around 90-120 mg/dl. After a two week recovery period, the diabetic monkeys are ready for the treatment trial.

The diabetic monkeys are treated with an islet cell differentiation transcription factor, such as the exemplary NeuroD or ngn3 with and without betacellulin using the exemplary HDAd as vector. In specific embodiment, the systemic dosage may be 1×10¹¹ to 5×10¹³ particles per kilogram body weight, although other dosages may be utilized. The animals are anesthetized for the treatment. One way to circumvent the toxicity of HDAd in monkeys is to inject it into the hepatic circulation after the liver blood supply is isolated. For example, the hepatic artery, vena cavas above and below the hepatic veins and the portal vein are clamped and the HDAd is delivered via the hepatic side of the clamped portal vein. When a vector comprising the present invention, such as HDAd, is delivered in this way the dosage needed is substantially (10 to 100 fold) lower than a systemic dosage stated above. The clamps remain in place for about 20-30 minutes. The residual HDAd in the hepatic circulation is then washed with buffered solution and the clamps are released. In this way, only a very small amount of the administered HDAd reaches the systemic circulation at a concentration that does not produce any significant toxicity. The animals are monitored during and after treatment, such as in terms of blood pressure, breathing, and/or by continuous EKG as for any operation under general anesthesia.

After the treatment, their blood glucose is monitored and they receive insulin therapy to keep the blood glucose below 200 mg/dl. For animals that respond to the treatment, the blood glucose gradually goes down to <200 mg/dl when exogenous insulin can be stopped; animals that have received the optimal dose develop a normal blood glucose. As a control for the treatment, the study is performed with an empty HDAd, i.e., a vector that does not contain a transgene (such as the exemplary NeuroD or ngn3) insert.

To test the effect and efficacy of treatment, one performs (1) an intravenous (IV) glucose tolerance test (GTT) in the treated and control monkeys to compare their blood glucose and blood insulin response to an IV glucose load; and/or (2) an oral GTT by giving them an oral glucose load. The blood glucose and insulin response is compared to that of nondiabetic monkeys. One may also perform necropsy on some treated and control animals to examine their liver for presence of islet cells that produce insulin and other islet hormones (glucagon, somatostatin and pancreatic polypeptide), such as by immunohistochemistry. A complete necropsy is performed to ensure that there are no major side effects of treatment. One may also assay for mRNA for insulin and other islet hormones as well as for other molecules intrinsic to beta cells by RT-PCR, as described elsewhere herein. The total amount of insulin in the liver is assayed, such as by an immunoassay. As part of the necropsy one may also examine the pancreas to ensure that at least the majority of the insulin comes from the newly formed islets in the liver and not from the pancreas. Although the above describes the studies using the exemplary HDAd as a vector, a similar approach can be used for any other vector that can be adopted for delivering the transcription factor(s).

### References

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### Patents

U.S. Pat. No. 6,210,960

U.S. Pat. No. 6,326,141

U.S. Pat. No. 5,328,986

U.S. Pat. No. 5,028,592

U.S. Pat. No. 4,554,101

U.S. Pat. No. 5,021,236

U.S. Pat. No. 4,472,509

U.S. Pat. No. 5,359,046

U.S. Pat. No. 3,791,932

U.S. Pat. No. 4,174,384

U.S. Pat. No. 3,949,064

U.S. Pat. No. 5,139,941

U.S. Pat. No. 4,797,368

WO 02/29010

### PUBLICATIONS

Ahlgren U, Pfaff S L, Jessell T M, Edlund T, Edlund H 1997 Independent requirement for ISLI in formation of pancreatic mesenchyme and islet cells. Nature 385:257-260.

Beale, E. G., Clouthier, D. E. & Hammer, R. E. Cell-specific expression of cytosolic phosphoenolpyruvate carboxykinase in transgenic mice. FASEB J. 6, 3330-3337 (1992).

Cabrele, C. et al. The first selective agonist for the neuorpeptide Y5 receptor increases food intake in rats. J. Biol. Chem. 275, 36043 (2000).

Docherty, K. Growth and development of the islets of Langerhans: implications for the treatment of diabetes mellitus. Curr Opin Pharmacol. 1(6), 641-50 (2001).

Dunbar, A. J. & Goddard, C. Structure-function and biological rold of betacellulin Int J Biochem & Cell Biol 32, 805-815 (2000).

Dutta et al., PDX:PBX complexes are required for normal proliferation of pancreatic cells during development. Proc Natl Acad Sci USA 98(3):1065-70 (2001).

Ferber, S. et al. Pancreatic and duodenal homeobox gene 1 induces expression of insulin genes in liver and ameliorates streptozotocin-induced hyperglycemia. Nat Med 6, 568-572 (2000).

Fujimiya, M., Mclntosh, C. H., Kimura, H. & Kwok, Y. N. Effect of carbachol on luminal release of somatostatin from isolated perfused rat duodenum. Neurosci Lett 145, 229-233 (1992).

Fujimiya, M., Okumiya, K. & Kuwahara, R. Immunoelectron microscopic study of the luminal release of serotonin from rat enterochromaffin cells induced by high intraluminal pressure. Histochem Cell Biol 108, 105-113 (1997).

Gradwohl G, Dierich A, LeMeur M, Guillemot F 2000 neurogenin3 is required for the development of the four endocrine cell lineages of the pancreas. Proc Natl Acad Sci USA 97:1607-1611

Gu, G., Dubauskaite, J. & Melton, D. A. Direct evidence for the pancreatic lineage:NGN3+cells are islet progenitors and are distinct from duct progenitors. Development 129, 2447-2457 (2002).

Halban, P. A., Kahn, S. E., Lernmark, A. & Rhodes, C. J. Gene and cell-replacement therapy in the treatment of type 1 diabetes. How high must the standards be set? Diabetes 50, 2181-2191 (2001).

Hansen L. et al., NeuroD/BETA2 gene varability and diabetes: no associations to late-onset type 2 diabetes but an A45 allele may represent a susceptibility marker for type I diabetes among Danes. Diabetes 49(5): 876-8 (2000).

Huang H. P., Chu K., Nemoz-Gaillard E., Elberg D., Tsai M. J. Neogenesis of beta-cells in adult BETA2/NeuroD-deficient mice. Mol Endocrinol 16(3): 541-51 (2002).

Huang et al., Regulation of the pancreatic islet-specific gene BETA2 (neuroD) by neurogenin 3. Mol Cell Biol 20(9): 3292-307 (2000).

Huotari et al., Growth factor-mediated proliferation and differentiation of insulin-producing INS-1 and RINm5F cells: identification of betacellulin as a novel beta-cell mitogen. Endocrinology 139(4): 1494-9 (1998).

Jonsson, J., Carlsson, L., Edlund, T. & Edlund, H. Insulin-promoter-factor 1 is required for pancreas development in mice. Nature 71, 606-609 (1994).

Kim, I.-H., Józkowicz, A., Piedra, P. A., Oka, K. & Chan, L. Lifetime correction of genetic deficiency in mice with a single injection of helper-dependent adenoviral vector. Proc Natl Acad Sci USA 98, 13282-13287 (2001).

Kochanek, S. High-capacity adenoviral vectors for gene transfer and somatic gene therapy. Hum Gene Therapy 10, 2451-2459 (1999).

Kojima, H. et al. Combined expression of pancreatic duodenal homeobox 1 and islet factor 1 induces immature enterocytes to produce insulin. Diabetes 51, 1398-1408 (2002).

Kojima et al. (2003) Nature Medicine 9: 596-603

Lawson et al., Genomic structure and promoter characterization of the gene encoding the ErbB ligand betacellulin. Biochim Biophys Acta 1576(1-2): 183-90 (2002).

Lee et al., Regulation of the pancreatic pro-endocrine gene neurogenin 3. Diabetes 50(6): 1512 (2001).

Lee J E, Hollenberg S M, Snider L, Turner D L, Lipnick N, Weintraub H 1995 Conversion of Xenopus ectoderm into neurons by NeuroD, a basic helix-loop-helix protein. Science 268:836-844

Lee C S, Perreault N, Brestelli J E, Kaestner K H. Neurogenin 3 is essential for the proper specification of gastric enteroendocrine cells and the maintenance of gastric epithelial cell identity. Genes Dev 16(12),1488-97 (2002).

Li et al., Promotion of beta-cell regeneration by betacellulin in ninety percent-pancreatectomized rats. Endocrinology 142(12):5379-85 (2001).

Li H, Arber S, Jessell T M, Edlund H 1999 Selective agenesis of the dorsal pancreas in mice lacking homeobox gene Hlxb9. Nat Genet 23:67-70

Liu H and Rice A P. (2000) Genomic organization and characterization of promoter function of the human CDK9 gene. Gene 252:51-59

Lozier, J. N., Metzger, M. E., Donahue, R. E. & Morgan, R. A. Adenovirus-mediated expression of human coagulation foactor IX in the rhesus macaque is associated with dose-limiting toxicity. Blood 94, 3968-3975 (1999).

Miyachi T., Maruyama H., Kitamura T., Nakamura S., Kawakami H. Structure and regulation of the human NeuroD (BETA2/BHF1) gene. Brain Res Mol Brain Res 69(2):223-31 (1999).

Miyata T, Maeda T, Lee J E 1999 NeuroD is required for differentiation of the granule cells in the cerebellum and hippocampus. Genes Dev 13:1647-1652

Mizushima, S. & Nagata, S. pEF-BOS, a powerful mammalian expression vector. Nucl Acids Res 18, 5322 (1990).

Mochizuki M., Amemiya S., Kobayashi K., Kobyashi K., Ishihara T., Aya M., Kato K., Kasuga A., Nakazawa S. The association of Ala45Thr polymorphism in NeuroD with child-onset type 1a diabetes in Japanese. Diabetes Res Clin Pract 55(1): 11-7 (2002).

Morral, N. et al. Administration of helper-dependent adenoviral vectors and sequential delivery of different vector serotypes for long-term liver-directed gene transfer in baboons. Proc Natl Acad Sci USA 96, 12816-12821 (1999).

Morral, N. et al. High doses of a helper-dependent adenoviral vector yield supraphysiological levels of α₁-antitrypsin with negligible toxicity. Hum Gene Therapy 9, 2709-2716 (1998).

Nakamura T., Kishi A., Nishio Y., Maegawa H., Egawa K., Wong N. C., Kojima H., Fujimiya M., Arai R., Kashiwagi A., Kikkawa R., Insulin production ina neuroectodermal tumor that expresses islet factor-1, but not pancreatic-duodenal homeobox-1. J Clin Endocrinol Metab 86(4):1795-800 (2001).

Naya, F. J. et al. Diabetes, defective pancreatic morphogenesis, and abnormal enteroendocrine differentiation in BETA2/NeuroD-deficient mice. Genes & Development 11, 2323-2334 (1997).

Naya F J, Stellrecht C M, Tsai M J 1995 Tissue-specific regulation of the insulin gene by a novel basic helix-loop-helix transcription factor. Genes Dev 9:1009-1019.

Ng et al. (2001) Development of a FLP/frt system for generating helper-dependent adenoviral vectors. Molecular Therapy 3: 809-815.

Offield M F, Jetton T L, Labosky P A, Ray M, Stein R W, Magnuson M A, Hogan B L, Wright C V 1996 PDX-1 is required for pancreatic outgrowth and differentiation of the rostral duodenum. Development 122:983-995

Oka, K. et al. Long-term stable correction of LDL receptor-deficient mice with a helper-dependent adenoviral vector expressing the VLDL receptor. Circulation 103, 1274-1281 (2001).

Umana et al. (2001) Efficient FPLe recombinase enables scalable production of helper-dependent adenoviral vectors with negligible helper-virus contamination. Nature Biotechnology 19: 582-585.

O'Neal, W. K. et al. Toxicological comparison of E2a-deleted and first-generation adenoviral vectors expressing alphal-antitrypsin after systemic delivery. Hum Gene Ther 9, 1587-98 (1998).

Parks, R. J. et al. A helper-dependent adenovirus vector system: Removal of helper virus by Cre-mediated excision of the viral packaging signal. Proc Natl Acad Sci USA 93, 13565-13570 (1996).

Pin, C. L., Rukstalis, J. M., Johnson, C. & Konieczny, S. F. The bHLH transcription factor mistl is required to maintain exocrine pancreas cell organization and acinar cell identity. J Cell Biol 155, 519-530 (2001).

Qin H and Gunning P. (1997) The 3′-end of the human beta-actin gene enhances activity of the beta-actin expression vector system: construction of improved vectors. J Biochem Biophys Methods 36(1):63-72.

Remington's Pharmaceutical Sciences, 13^{th} and 18^{th} editions, Maek Printing Company, 1990.

Schmied et aL Differentiation of islet cells in long-term culture. Pancreas 20(4): 337-47 (2000).

Schwab M H, Bartholomae A, Heimrich B, Feldmeyer D, Druffel-Augustin S, Goebbels S, Naya F J, Zhao S, Frotscher M, Tsai M J, Nave K A 2000 Neuronal basic helix-loop-helix proteins (NEX and BETA2/Neuro D) regulate terminal granule cell differentiation in the hippocampus. J Neurosci 20:3714-3724

Schwitzgebel V M, Scheel D W, Conners J R, Kalamaras J, Lee J E, Anderson D J, Sussel L, Johnson J D, German M S. Expression of neurogenin3 reveals an islet cell precursor population in the pancreas. Development 127(16), 3533-42 (2000).

Shing, Y. et al. Betacellulin: a mitogen from pancreatic beta-cell tumors. Science 259, 2681-2689 (1993).

Slack, J. M. W. Developmental biology of the pancreas. Development 121, 1569-1580 (1995).

Sosa-Pineda B, Chowdhury K, Torres M, Oliver G, Gruss P 1997 The Pax4 gene is essential for differentiation of insulin-producing_cells in the mammalian pancreas. Nature 386:399-402

St-Onge L, Sosa-Pineda B, Chowdhury K, Mansouri A, Gruss P 1997 Pax6 is required for differentiation of glucagon-producing_-cells in mouse pancreas. Nature 387:406-409

Sussel L, Kalamaras J, Hartigan-O'Connor D J, Meneses J J, Pedersen R A, Rubenstein J L, German M S 1998 Mice lacking the homeodomain transcription factor Nkx2.2 have diabetes due to arrested differentiation of pancreatic cells. Development 125:2213-2221

Theise, N. D. et al. The canals of hering and hepatic stem cells in humans. Hepatology 30, 1425-1433 (1999).

Watada, H. et al. PDX-1 induces insulin and glucokinase gene expressions in □TC1 clone 6 cells in the presence of betacellulin. Diabetes 45, 1826-1831 (1996).

Watari, N. & Hotta, Y. in Endocrine Gut and Pancreas (ed Fujita, T.) 179-184 (Elsevier Sci Publ Co, Amsterdam, 1975).

Yang, L. et al. In vitro trans-differentiation of adult hepatic stem cells into pancreatic endocrine hormone-producing cells. Proc Natl Acad Sci USA 99, 8078-8083 (2002).

Yoon, J. W. & Jun, H. S. Recent advances in insulin gene therapy for type 1 diabetes Trends Mol Med 8, 62-68 (2002).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method of treating a mammal for insulin-dependent diabetes comprising delivering to the mammal a composition comprising an effective amount of an islet cell differentiation transcription factor polypeptide or of a nucleic acid expressing the islet cell differentiation transcription factor polypeptide, wherein the factor promotes normalization of insulin level in the mammal to treat the insulin-dependent diabetes.

2. The method of claim 1, wherein said delivering of the composition is in vivo.

3. The method of claim 1, wherein said delivering of the composition to the mammal is further defined as:
introducing the composition into a somatic mammalian cell ex vivo; and
delivering the cell comprising the composition to the individual.

4. The method of claim 1, wherein the composition is in a pharmaceutically acceptable diluent.

5. The method of claim 1, wherein the islet cell differentiation transcription factor polypeptide is NeuroD, ngn3, Pax6, Pax4, Nkx2.2, Nkx6.1, Is1-1, or a combination thereof.

6. The method of claim 3, wherein the islet cell differentiation transcription factor is NeuroD.

7. The method of claim 3, wherein the islet cell differentiation transcription factor is ngn3.

8. The method of claim 1, further comprising administering a betacellulin polypeptide or a nucleic acid expressing the betacellulin polypeptide to the mammal.

9. The method of claim 8, wherein the betacellulin polypeptide and the islet cell differentiation factor polypeptide are co-administered to the mammal.

10. The method of claim 8, wherein the betacellulin polypeptide and the islet cell differentiation factor polypeptide are in the same pharmaceutically acceptable diluent.

11. The method of claim 8, wherein the betacellulin polypeptide is on the same molecule as the islet cell differentiation transcription factor polypeptide.

12. The method of claim 8, wherein the nucleic acid expressing the betacellulin polypeptide is on the same molecule as the nucleic acid expressing the islet cell differentation transcription factor polynucleotide.

13. The method of claim 1, further comprising administering a Pdx-1 polypeptide or a nucleic acid expressing the Pdx-1 polypeptide to the mammal.

14. The method of claim 13, wherein the Pdx-1 polypeptide and the islet cell differentiation factor polypeptide are co-administered to the mammal.

15. The method of claim 1, wherein the nucleic acid comprises an expression vector.

16. The method of claim 15, wherein the expression vector is a non-viral vector.

17. The method of claim 15, wherein the expression vector is a viral vector.

18. The method of claim 17, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

19. The method of claim 18, wherein the viral vector is an adenoviral vector.

20. The method of claim 19, wherein the adenoviral vector is helper dependent.

21. The method of claim 17, wherein the viral vector is administered at between about 10¹¹ to about 10¹² viral particles.

22. The method of claim 21, wherein the viral vector is administered at between about 1×10¹¹ to about 5×10¹¹ viral particles.

23. The method of claim 15, wherein the expression vector further comprises a promoter operable in a eukaryotic cell.

24. The method of claim 23, wherein the promoter is a tissue-specific promoter.

25. The method of claim 1, wherein the composition is administered systemically by continuous infusion or by intravenous injection.

26. The method of claim 1, wherein the composition is injectable.

27. The method of claim 26, wherein the composition is administered intraperitoneally or intraportally.

28. A method of increasing an insulin level in a somatic cell comprising delivering to the cell a composition comprising an islet cell differentiation transcription factor polypeptide or a nucleic acid expressing the islet cell differentiation transcription factor polypeptide, wherein the presence of the polypeptide effects an increase in the insulin level in the cell.

29. The method of claim 28, wherein said delivering of the composition is in vivo.

30. The method of claim 28, wherein said delivering of the composition is in vitro.

31. The method of claim 28, wherein the somatic cell is a hepatic cell, a pancreatic cell, a skeletal muscle cell, an adipose tissue cell, a stem cell, or a progenitor cell.

32. The method of claim 28, wherein the stem cell is a hematopoietic cell, a pluripotent cell or a totipotent cell.

33. The method of claim 32, wherein the stem cell is a pluripotent cell.

34. The method of claim 32, wherein the islet cell differentiation transcription factor polypeptide is NeuroD, ngn3, Pax6, Pax4, Nkx2.3, Nkx6.1, Is1-1 or a combination thereof.

35. The method of claim 34, wherein the islet cell differentiation transcription factor is NeuroD.

36. The method of claim 34, wherein the islet cell differentiation transcription factor is ngn3.

37. The method of claim 28, wherein the composition further comprises a betacellulin polypeptide or a nucleic acid expressing the betacellulin polypeptide.

38. The method of claim 28, wherein the composition further comprises a Pdx-1 polypeptide or a nucleic acid expressing the Pdx-1 polypeptide.

39. The method of claim 28, wherein the nucleic acid comprises an expression vector.

40. The method of claim 39, wherein the expression vector is a non-viral vector.

41. The method of claim 39, wherein the expression vector is a viral vector.

42. The method of claim 41, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

43. The method of claim 41, wherein the viral vector is an adenoviral vector.

44. The method of claim 43, wherein the adenoviral vector is helper dependent.

45. The method of claim 41, wherein the viral vector is administered at between about 10¹¹ to about 10¹² viral particles.

46. The method of claim 45, wherein the viral vector is administered at between about 1×10¹¹ to about 5×10¹¹ viral particles.

47. The method of claim 39, wherein expression vector further comprises a promoter operable in a eukaryotic cell.

48. The method of claim 47, wherein the promoter is a tissue-specific promoter.

49. A method of generating an insulin-producing cell comprising delivering to a somatic cell a composition comprising an islet cell differentiation factor polypeptide or a nucleic acid expressing the islet cell differentiation factor polypeptide, wherein the presence of the factor effects the generation of an insulin-producing cell from the somatic cell.

50. The method of claim 49, wherein said delivering of the composition is in vivo.

51. The method of claim 49, wherein said delivering of the composition is in vitro.

52. The method of claim 49, wherein the somatic cell is a hepatic cell, a pancreatic cell, a skeletal muscle cell, an adipose tissue cell, a stem cell, or a progenitor cell.

53. The method of claim 52, wherein the stem cell is a hematopoietic cell, a pluripotent cell or a totipotent cell.

54. The method of claim 52, wherein the stem cell is a pluripotent cell.

55. The method of claim 49, wherein the islet cell differentiation transcription factor polypeptide is NeuroD, ngn3, Pax6, Pax4, Nkx2.3, Nkx6.1, Is1-1, or a combination thereof.

56. The method of claim 60, wherein the islet cell differentiation transcription factor is NeuroD.

57. The method of claim 60, wherein the islet cell differentiation transcription factor is ngn3.

58. The method of claim 49, wherein the composition further comprises a betacellulin polypeptide or a nucleic acid expressing the betacellulin polypeptide.

59. The method of claim 49, wherein the composition further comprises a Pdx-1 polypeptide or a nucleic acid expressing the Pdx-1 polypeptide.

60. The method of claim 49, wherein the nucleic acid comprises an expression vector.

61. The method of claim 60, wherein the expression vector is a non-viral vector.

62. The method of claim 60, wherein the expression vector is a viral vector.

63. The method of claim 62, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

64. The method of claim 62, wherein the viral vector is an adenoviral vector.

65. The method of claim 64, wherein the adenoviral vector is helper dependent.

66. The method of claim 62, wherein the viral vector is administered at between about 10¹¹ to about 10¹² viral particles.

67. The method of claim 66, wherein the viral vector is administered at between about 1×10¹¹ to about 5×10¹¹ viral particles.

68. The method of claim 60, wherein the expression vector further comprises a promoter operable in a eukaryotic cell.

69. The method of claim 68, wherein the promoter is a tissue-specific promoter.

70. The method of claim 49, wherein a plurality of insulin-producing cells are generated.

71. The method of claim 70, wherein at least one insulin-producing cell in the plurality is characterized by one or more secretory granules in the cytoplasm.

72. The method of claim 71, wherein each of the plurality of secretory granules comprise a diameter of about 300 nm to about 600 nm.

73. The method of claim 71, wherein each of the plurality of secretory granules comprises an insulin polypeptide.

74. A therapeutic composition comprising an isolated islet cell differentiation transcription factor polypeptide and/or an isolated nucleic acid expressing the polypeptide.

75. The composition of claim 74, wherein said islet cell differentiation transcription factor is NeuroD.

76. The composition of claim 74, wherein said islet cell differentiation transcription factor is ngn3.

77. The composition of claim 74, wherein the composition is in a pharmaceutically acceptable diluent.

78. The composition of claim 74, wherein the nucleic acid is an expression vector.

79. The composition of claim 78, wherein the expression vector is a non-viral vector.

80. The composition of claim 78, wherein the expression vector is a viral vector.

81. The composition of claim 80, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

82. The composition of claim 80, wherein the viral vector is an adenoviral vector.

83. The composition of claim 82, wherein the adenoviral vector is helper dependent.

84. The composition of claim 80, wherein the composition comprises between about 10¹¹ to about 10¹² viral particles.

85. The composition of claim 74, wherein the composition further comprises an isolated betacellulin polypeptide or an isolated nucleic acid expressing the betacellulin polypeptide.

86. The composition of claim 85, wherein the nucleic acid is an expression vector.

87. The composition of claim 86, wherein the expression vector is a non-viral vector.

88. The composition of claim 86, wherein the expression vector is a viral vector.

89. The composition of claim 88, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

90. The composition of claim 86, wherein the expression vector further comprises a promoter operable in a eukaryotic cell.

91. The composition of claim 90, wherein the promoter is a tissue-specific promoter.

92. The method of claim 31, wherein the progenitor cell is from skeletal muscle tissue, hepatic tissue, adipose tissue, or pancreatic tissue.

93. The method of claim 52, wherein the progenitor cell is from skeletal muscle tissue, hepatic tissue, adipose tissue, or pancreatic tissue.

94. An insulin-producing cell comprising a vector, said vector comprising nucleic acid sequence encoding an islet cell differentiation transcription factor.

95. The cell of claim 94, wherein said cell further comprises a vector comprising nucleic acid sequence encoding betacellulin.

96. The cell of claim 94, wherein said cell is in a pancreatic islet.

97. The cell of claim 96, wherein said pancreatic islet is in a liver.

98. An insulin-producing cell generated by the method comprising:
obtaining a somatic cell; and
transfecting said cell with a vector comprising nucleic acid sequence encoding an islet cell differentiation transcription factor, wherein upon said transfecting step said cell produces insulin.

99. The cell of claim 98, wherein said insulin-producing cell is further defined as a beta cell.

100. The cell of claim 98, wherein said insulin-producing cell is comprised in a pancreatic islet in vivo.

101. The cell of claim 98, wherein said insulin-producing cell is in the liver.

102. The cell of claim 100, wherein said islet is in the liver.

103. A method of generating at least one pancreatic islet, comprising:
providing at least one somatic cell; and
transfecting an effective amount of an islet cell differentiation transcription factor polypeptide or a nucleic acid expressing the islet cell differentiation transcription factor polypeptide into said cell, wherein upon said transfecting step said at least one pancreatic islet is generated.

104. The method of claim 103, wherein said pancreatic islet is generated in liver tissue.

105. The method of claim 103, wherein said pancreatic islet is generated in vitro.

106. The method of claim 103, wherein said pancreatic islet is generated in vivo.

107. The method of claim 103, wherein said somatic cell is a hepatic cell, a pancreatic cell, a skeletal muscle cell, an adipose tissue cell, a stem cell, or a progenitor cell.

108. The method of claim 103, wherein said islet cell differentiation transcription factor is NeuroD, ngn3, Pax6, Pax4, Nkx2.2, Nkx6.1, Is1-1, or a combination thereof.

109. A use of a sequence for the treatment of type 1 or type 2 diabetes, said sequence having a region selected from the group consisting of SEQ ID NO:1 through SEQ ID NO:67, SEQ ID NO:79, and SEQ ID NO:83 through SEQ ID NO:93.

110. A composition comprising:
NeuroD polypeptide or a polynucleotide expressing a NeuroD polypeptide; and
betacellulin polypeptide or a polynucleotide expressing a betacellulin polypeptide.

111. The composition of claim 110, wherein said composition further comprises a pharmaceutically acceptable diluent.

112. A composition comprising:
ngn3 polypeptide or a polynucleotide expressing a ngn3 polypeptide; and
betacellulin polypeptide or a polynucleotide expressing a betacellulin polypeptide.

113. The composition of claim 112, wherein said composition further comprises a pharmaceutically acceptable diluent.


SEQUENCE LISTING (194 sequences)

SEQ ID NO: 1 - protein, 356 aa, Homo sapiens [sequence omitted]

SEQ ID NO: 2 - protein, 244 aa, Mus musculus
MPAPLETCISDLDCSSSNSSSDLSSFLTDEEDCARLQPLASTSGLSVPARRSAPALSGASNVPGAQDEEQERRRRRGRARVRSEALLHSLRRSRRVKANDRERNRMHNLNAALDALRSVLPSFPDDTKLTKIETLRFAYNYIWALAETLRLADQGLPGGSARERLLPPQCVPCLPGPPSPASDTESWGSGAAASPCATVASPLSDPSSPSASEDFTYGPGDPLFSFPGLPKDLLHTTPCFIPYH

SEQ ID NO: 3 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 4 - protein, 357 aa, Rattus norvegicus
MTKSYSESGLMGEPQPQGPPSWTDECLSSQDEEHEADKKEDELEAMNAEEDSLRNGGEEEDEDEDLEEEEEEEEEEDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSTEFEKNYAFTMHYPAATLAGPQSHGSIFSSGAAAPRCEIPIDNIMSFDSHSHHERVMSAQLNAIFHD

SEQ ID NO: 5 - protein, 357 aa, Gallus gallus
MTKSYSESGPAGEPQAQAPPGWAAGCLSPPADGPEADKKEEDLEALHGEAEEDALRNGEEEDEEDELDEEEEEEEEEEDDEQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQSADAAPHLPPAGAPFAPPPFPYASPGLPSPPYGTMDSSHLFHLKPPHAYGAALEPFFEGGLPEGAGPAFDGPLSPPLSINGNFSFKHEPAADFDKSYAFTMHYPAGPLPAAPAHAAVFSGAAARCELPGDGLAPYEGHPHHERVLSAQLSAIFHE

SEQ ID NO: 6 - protein, 352 aa, Xenopus laevis
MTKSYGENGLILAETPGCRGWVDECLSSQDENDLEKKEGELMKEDDEDSLNHHNGEENEEEDEGDEEEEDDEDDDEDDDQKPKRRGPKKKKMTKARVERFKVRRMKANARERNRMHGLNDALDSLRKVVPCYSKTQKLSKIETLRLAKNYIWALSEILRSGKSPDLVSFVQTLCKGLSQPTTNLVAGCLQLNPRTFLPEQSQDIQSHMQTASSSFPLQGYPYQSPGLPSPPYGTMDSSHVFHVKPHSYGAALEPFFDSSTVTECTSPSFDGPLSPPLSVNGNFTFKHEHSEYDKNYTFTMHYPAATISQGHGPLFSTGGPRCEIPIDTIMSYDGHSHHERVMSAQLNAIFHD

SEQ ID NO: 7 - protein, 357 aa, Rattus norvegicus; identical to SEQ ID NO: 4

SEQ ID NO: 8 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 9 - protein, 355 aa, Mesocricetus auratus
MTKSYSESGLMGEPQPQGPPSWTDECLSSQDEDHEADKKEDELEAMNAEEDSLRNGGDEEDEDEDLEEEDEEEEEDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFQVKPPPHAYSATLEPFFESPLTDCTSPSFDGPLSPPLSINGNFSFKHEPSAEFEKNYAFTMHYPAATLAGPQSHGSIFSGATAPRCEIPIDNIMSFDSHSHHERVMSAQLNAIFHD

SEQ ID NO: 10 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 11 - protein, 350 aa, Danio rerio
MTKSYSEESMMLESQSSSNWTDKCHSSSQDERDVDKTSEPMLNDMEDDDDAGLNRLEDEDDEEEEEEEEDGDDTKPKRRGPKKKKMTKARMQRFKMRRMKANARERNRMHGLNDALESLRKVVPCYSKTQKLSKIETLRLAKNYIWALSEILRSGKSPDLMSFVQALCKGLSQPTTNLVAGCLQLNPRTFLPEQSQEMPPHMQTASASFSALPYSYQTPGLPSPPYGTMDSSHIFHVKPHAYGSALEPFFDTTLTDCTSPSFDGPLSPPLSVNGNFSFKHEPSSEFEKNYAFTMHYQAAGLAGAQGHAASLYAGSTQRCDIPMENIMSYDGHSHHERVMNAQLNAIFHDS

SEQ ID NO: 12 - protein, 350 aa, Danio rerio; identical to SEQ ID NO: 11

SEQ ID NO: 13 - protein, 350 aa, Danio rerio; identical to SEQ ID NO: 11

SEQ ID NO: 14 - protein, 357 aa, Mus musculus; identical to SEQ ID NO: 8

SEQ ID NO: 15 - protein, 383 aa, Mus musculus
MLTRLFSEPGLLSDVPKFASWGDGDDDEPRSDKGDAPPQPPPAPGSGAPGPARAAKPVSLRGGEEIPEPTLAEVKEEGELGGEEEEEEEEEEGLDEAEGERPKKRGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGADGAGRFHGSGGPFAMHPYPYPCSRLAGAQCQAAGGLGGGAAHALRTHGYCAAYETLYAAAGGGGASPDYNSSEYEGPLSPPLCLNGNFSLKQDSSPDHEKSYHYSMHYSALPGSRPTGHGLVFGSSAVRGGVHSENLLSYDMHLHHDRGPMYEELNAFFHN

SEQ ID NO: 16 - protein, 352 aa, Xenopus laevis; identical to SEQ ID NO: 6

SEQ ID NO: 17 - protein, 186 aa, Sus scrofa
DLRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGAAAPRCEIPIDNIMSFDSHSH

SEQ ID NO: 18 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVGGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 19 - protein, 381 aa, Homo sapiens [sequence omitted]

SEQ ID NO: 20 - protein, 356 aa, Homo sapiens; identical to SEQ ID NO: 3

SEQ ID NO: 21 - protein, 356 aa, Homo sapiens; identical to SEQ ID NO: 18

SEQ ID NO: 22 - protein, 357 aa, Rattus norvegicus; identical to SEQ ID NO: 4

SEQ ID NO: 23 - protein, 216 aa, Eleutherodactylus coqui
KRRGPKKKKMTKARVERFKMRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHMQAASASFPLHPYPYQSPGLPSPPYGTMDSSHIFQVKPHSYGVALEPFFESTVTDCTSPSFDGPLSPPLSVNGNFSFKHEPSAEFDKNYAF

SEQ ID NO: 24 - protein, 356 aa, Homo sapiens; identical to SEQ ID NO: 18

SEQ ID NO: 25 - protein, 357 aa, Rattus norvegicus; identical to SEQ ID NO: 4

SEQ ID NO: 26 - protein, 357 aa, Gallus gallus
MTKSYSESGPAGEPQAQAPPGWAAGCLSPPADGPEADKKEEDLEALHGEAEEDALRNGEEEDEEDELDEEEEEEEEEEDDEQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQSADAAPHLPPAGAPFAPPPFPYASPGLPSPPYGTMDSSHLFHLKPPHAYGAALEPFFEGGLPEGAGPAFDGPLSPPLSIYGNFSFKHEPAADFDNSYAFTMHYPAGPLPAAPAHAAVFSGAAARCELPADGLAPYEGHPHHERVLSAQLSAIFHE

SEQ ID NO: 27 - protein, 352 aa, Xenopus laevis; identical to SEQ ID NO: 6

SEQ ID NO: 28 - protein, 121 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAAL

SEQ ID NO: 29 - protein, 285 aa, Mus musculus
EEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 30 - protein, 113 aa, Homo sapiens
KKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQN

SEQ ID NO: 31 - protein, 382 aa, Gallus gallus
MLTRLFSEPGLLSDVPKFASWGDGEDDEPRSDKGDAPPPPPPAPGPGAPGPARAAKPVPLRGEEGTEATLAEVKEEGELGGEEEEEEEEEEGLDEAEGERPKKRGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGADGAGRFHGSGGPFAMHPYPYPCSRLAGAQCQAAGGLGGGAAHALRTHGYCAAYETLYAAAGGGGASPDYNSSEYEGPLSPPLCLNGNFSLKQDSSPDHEKSYHYSMHYSALPGSRPTGHGLVFGSSAVRGGVHSENLLSYDMHLHHDRGPMYEELNAFFHN

SEQ ID NO: 32 - protein, 357 aa, Artificial Sequence; identical to SEQ ID NO: 5
  Description of Artificial Sequence  Synthetic Peptide

SEQ ID NO: 33 - protein, 380 aa, Artificial Sequence
  Description of Artificial Sequence  Synthetic Peptide
MLTRLFSEPGLLSDVPKFASWGDGDDDEPRSDKGDAPPQPSPAPGSGAPGPARAAKPVSLRGGEEIPEPTLAEVKEEGELGGEEEEEEEEEEGLDEAEGERPKKRGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGADGGRFHGSGGPFAMHPYPYPCSRLAGHSVRRLAAWAEXGARLRTHGYCAAYETLYAAAGGGGASPDYNSSEYEGPLSPPLCLNGNFSLKQDSSPDHEKSYHYSMHYSRCPGSRHGHGLVFGSSAVRGGVHSENLLSYDMHLHHDRGPMYEELNAFFHN

SEQ ID NO: 34 - protein, 356 aa, Artificial Sequence
  Description of Artificial Sequence  Synthetic Peptide
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPXYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 35 - protein, 103 aa, Artificial Sequence
  Description of Artificial Sequence  Synthetic Peptide
PGVLRSRGTGRRAGEAAAAGRXSLRGAAAXAAQERRVKANDRERNRMHNLNAALDALRSVLPSFPDDTKLTKIESLRXAYNYIWALAETLRWRXKGCPEAVPG

SEQ ID NO: 36 - protein, 379 aa, Artificial Sequence
  Description of Artificial Sequence  Synthetic Peptide
MLTRLFSEPGLLSDVPKFASWGDGEDDEPRSDKGDAPPPPPPAPGPGAPGPARAAKPVPLRGEEGTEATLAEVKEEGELGGEEEEEEEEEEGLDEAEGERPKKRGPKKRKMTKARLERSKLRRQKANARERNRMHDLNAALDNLRKVVPCYSKTQKLSKIETLRLAKNYIWALSEILRSGKRPDLVSYVQTLCKGLSQPTTNLVAGCLQLNSRNFLTEQGRDGAXRFHGSGGPFAMHPYPYPCSRGGRTVPGAAAWAAAGARLRTHGYCAAYETLYAAAGGGGASPDYNSSEYEGPLSPPLCLNGNFSLKQDSSPDHEKSYHYSMHYSGCPGSRHGHGLVFGSSAVRGGVHSENLLSYDMHLHHXRGPMXXELNAFFHN

SEQ ID NO: 37 - protein, 156 aa, Artificial Sequence
  Description of Artificial Sequence  Synthetic Peptide
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEI

SEQ ID NO: 38 - protein, 352 aa, Artificial Sequence; identical to SEQ ID NO: 6
  Description of Artificial Sequence Synthetic Peptide

SEQ ID NO: 39 - protein, 357 aa, Mus musculus; identical to SEQ ID NO: 8

SEQ ID NO: 40 - protein, 383 aa, Mus musculus; identical to SEQ ID NO: 15

SEQ ID NO: 41 - protein, 113 aa, Rattus norvegicus; identical to SEQ ID NO: 30

SEQ ID NO: 42 - protein, 237 aa, Homo sapiens
MPARLETCISDLDCASSSGSDLSGFLTDEEDCARLQQAASASGPPAPARRSAPNISRASEVPGAQDDEQERRRRRGRTRVRSEALLHSLRRSRRVKANDRERNRMHNLNAALDALRSVLPSFPDDTKLTKIETLRFAYNYIWALAETLRLADQGLPGGGARERLLPPQCVPCLPGPPSPASDAESWGSGAAAASPLSDPSSPAASEDFTYRPGDPVFSFPSLPKDLLHTTPCFIPYH

SEQ ID NO: 43 - protein, 352 aa, Xenopus laevis; identical to SEQ ID NO: 6

SEQ ID NO: 44 - protein, 52 aa, Homo sapiens
KNYIWALSEILRSGKSPDLVSFVQTLCKGLSQPTTNLVAGCLQLNPRTFLPE

SEQ ID NO: 45 - protein, 214 aa, Homo sapiens [sequence omitted]

SEQ ID NO: 46 - protein, 214 aa, Mus musculus
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 47 - protein, 214 aa, Homo sapiens
MTPQPSGAPTVQVTRETERSFPRASEDEVTCPTSAPPSPTRTPGNCAEAEEGGCRGAPRKLRARRGGRSRPKSELALSKQRRSRRKKANDRERNRMHDLNSALDALRGVLPTFPDDAKLTKIETLRFAHNYIWALTQTLRIADHSLYALEPPAPHCGELGSPGGPPGDWGSLYSPVSQAGSLSPAASLEERPGLLGATSSACLSPGSLAFSDFL

SEQ ID NO: 48 - protein, 214 aa, Mus musculus; identical to SEQ ID NO: 46

SEQ ID NO: 49 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 50 - protein, 208 aa, Danio rerio
MTPRSSCALVGRNGTFKSNWSSASEPKFGSTDMTKSQPIKYNREAELASKEWSFTFREDKTSNGKLKKLMSTSRQRGNRRVKANDRGRHRMHNLNSALDNLRSVLPTFPDDAKLTKIETLRFARNYIWALSETLRIADHVRQRSNHAQDQENLAVPNACLDVRYGASSACASKWHSTNSSSNWQETQGFYTDLLLEEFNGNFQDNLTF

SEQ ID NO: 51 - protein, 214 aa, Homo sapiens; identical to SEQ ID NO: 47

SEQ ID NO: 52 - protein, 214 aa, Mus musculus; identical to SEQ ID NO: 46

SEQ ID NO: 53 - protein, 214 aa, Homo sapiens; identical to SEQ ID NO: 47

SEQ ID NO: 54 - protein, 214 aa, Mus musculus; identical to SEQ ID NO: 46

SEQ ID NO: 55 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 56 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 57 - protein, 246 aa, Danio rerio
MNREEHYYPPNHLYKDSCAFQRHPNEDYSQNPPPCLYMRQAHSVYASPLGAQDQPNLTDITSYNMSSRDDPAGPHLHLPQTSQTSLQSLGGYGDSLDLCGDRNRYHLPFPWMKSTKSHTHAWKGQWTGPYMVEAEENKRTRTAYTRAQLLELEKEFLFNKYISRPRRVELALTLSLTERHIKIWFQNRRMKWKKEEDKRRARGVDPEQDSSITSGDLKDESCVGTATLAGPPSPLHPHAPSVQQDS

SEQ ID NO: 58 - protein, 283 aa, Rattus norvegicus
MNSEEQYYAATQLYKDPCAFQRGPVPEFSANPPACLYMGRQPPPPPTPQFAGSLGTLEQGSPPDISPYEVPPLADDPAGAHLHHHLPAQLGLAHPPPGPFPNGTETGGLEEPSRVHLPFPWMKSTKAHAWKSQWAGGAYAAEPEENKRTRTAYTRAQLLELEKEFLFNKYISRPRRVELAVMLNLTERHIKIWFQNRRMKWKKEEDKKRSSGTTSGGGGGEEPEQDCAVTSGEELLALPKPPPPGGVVPSGVPAAAREGRLPSGLSASPQPSSIAPLRPQEPR

SEQ ID NO: 59 - protein, 283 aa, Mesocricetus auratus
MNGEEQYYAATQLYKDPCAFQRGPVPEFSANPPACLYMGRQPPPPPPPQFAGALGTLEQGSPPDISPYEVPPLAEDPAVAHLHHHLPAQLGLAHPPSGPFPNGTEPGGLEEPSRGQLPFPWMKSTKAHAWKGQWAGGAYAVEPEENKRTRTAYTRAQLLELEKEFLFNKYISRPRRVELAVMLNLTERHIKIWFQNRRMKWKKEEDKKRSSGTASGGVGGDEPEQDSAVTSGEELLALPPPPPPGGAVPPGVPAAAREGRLPPGLSASPQPSSIAPRRPQEPR

SEQ ID NO: 60 - protein, 283 aa, Rattus norvegicus
MNSEEQYYAATQLYKDPCAFQRGPVPEFSANPPACLYMGRQPPPPPPPQFAGSLGTLEQGSPPDISPYEVPPLADDPAGAHLHHHLPAQLGLAHPPPGPFPNGTETGGLEEPSRVHLPFPWMKSTKAHAWKSQWAGGAYAAEPEENKRTRTAYTRAQLLELEKEFLFNKYISRPRRVELAVMLNLTERHIKIWFQNRRMKWKKEEDKKRSSGTTSGGGGGEEPEQDCAVTSGEELLALPPPPPPGGAVPSGVPAAAREGRLPSGLSASPQPSSIAPLRPQEPR

SEQ ID NO: 61 - protein, 284 aa, Mus musculus; identical to SEQ ID NO: 56

SEQ ID NO: 62 - protein, 283 aa, Homo sapiens; identical to SEQ ID NO: 55

SEQ ID NO: 63 - protein, 284 aa, Mus musculus; identical to SEQ ID NO: 56

SEQ ID NO: 64 - protein, 284 aa, Mus musculus; identical to SEQ ID NO: 56

SEQ ID NO: 65 - protein, 284 aa, Mus musculus; identical to SEQ ID NO: 56

SEQ ID NO: 66 - protein, 283 aa, Homo sapiens; identical to SEQ ID NO: 55

SEQ ID NO: 67 - protein, 283 aa, Mesocricetus auratus; identical to SEQ ID NO: 59

SEQ ID NO: 68 - protein, 69 aa, Homo sapiens
MYLLFICNFSLCYYFLIRTLIICILSSNWEKSNWLGSNNRREISITFHLSIVTRITSQTKKKSRKEVRS

SEQ ID NO: 69 - protein, 177 aa, Mus musculus
MDPTAPGSSVSSLPLLLVLALGLAILHCVVADGNTTRTPETNGSLCGAPGENCTGTTPRQKVKTHFSRCPKQYKHYCIHGRCRFVVDEQTPSCICEKGYFGARCERVDLFYLQQDRGQILVVCLIVVMVVFIILVIGVCTCCHPLRKHRKKKKEEKMETLDKDKTPISEDIQETNIA

SEQ ID NO: 70 - protein, 177 aa, Rattus norvegicus
MDSTAPGSGVSSLPLLLALVLGLVILQCVVADGNTTRTPETNGSLCGAPGENCTGTTPRQKSKTHFSRCPKQYKHYCIHGRCRFVMDEQTPSCICEKGYFGARCEQVDLFYLQQDRGQILVVCLIGVMVLFIILVIGVCTCCHPLRKHRKKKKEEKMETLSKDKTPISEDIQETNIA

SEQ ID NO: 71 - protein, 177 aa, Mus musculus; identical to SEQ ID NO: 69

SEQ ID NO: 72 - protein, 50 aa, Mus musculus
MVVFIILVIGVCTCCHPLRKHRKKKKEEKMETLDKDKTPISEDIQETNIA

SEQ ID NO: 73 - protein, 177 aa, Mus musculus; identical to SEQ ID NO: 69

SEQ ID NO: 74 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 75 - protein, 177 aa, Rattus norvegicus; identical to SEQ ID NO: 70

SEQ ID NO: 76 - protein, 178 aa, Bos taurus
MARAAPGSGASPLPLLPALALGLVILHCVVADGNSTRSPEDDGLLCGDHAENCPATTTQPKRRGHFSRCPKQYKHYCIKGRCRFVVAEQTPSCVCDEGYAGARCERVDLFYLRGDRGQILVICLIAVMVIFIILVVSICTCCHPLRKRRKRRKKEEEMETLGKDITPINDDIQETSIA

SEQ ID NO: 77 - protein, 178 aa, Homo sapiens; identical to SEQ ID NO: 74

SEQ ID NO: 78 - protein, 177 aa, Mus musculus; identical to SEQ ID NO: 69

SEQ ID NO: 79 - protein, 52 aa, Homo sapiens; identical to SEQ ID NO: 44

SEQ ID NO: 80 - protein, 178 aa, Bos taurus; identical to SEQ ID NO: 76

SEQ ID NO: 81 - protein, 177 aa, Mus musculus; identical to SEQ ID NO: 69

SEQ ID NO: 82 - protein, 178 aa, Homo sapiens; identical to SEQ ID NO: 74

SEQ ID NO: 83 - protein, 343 aa, Homo sapiens
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCPLASLSGSQALLWPGCPLLYGLE

SEQ ID NO: 84 - protein, 349 aa, Mus musculus
MQQDGLSSVNQLGGLFVNGRPLPLDTRQQIVQLAIRGMRPCDISRSLKVSNGCVSKILGRYYRTGVLEPKCIGGSKPRLATPAVVARIAQLKDEYPALFAWEIQHQLCTEGLCTQDKAPSVSSINRVLRALQEDQSLHWTQLRSPAVLAPVLPSPHSNCGAPRGPHPGTSHRNRTIFSPGQAEALEKEFQRGQYPDSVARGKLAAATSLPEDTVRVWFSNRRAKWRRQEKLKWEAQLPGASQDLTVPKNSPGIISAQQSPGSVPSAALPVLEPLSPPFCQLCCGTAPGRCSSDTSSQAYLQPYWDCQSLLPVASSSYVEFAWPCLTTHPVHHLIGGPGQVPSTHCSNWP

SEQ ID NO: 85 - protein, 422 aa, Homo sapiens [sequence omitted]

SEQ ID NO: 86 - protein, 102 aa, Mus musculus
MQNSHSGVNQLGGVFVNGRPLPDSTRQKIVELAHSGARPCDISRILQTHADAKVQVLDNENVSNGCVSKILGRYYETGSIRPRAIGGSKPRVATPEVVSKIA

SEQ ID NO: 87 - protein, 273 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 88 - protein, 87 aa, Mus musculus
MSLTNTKDGVFKVKDILDLPDTNDEDGSVAEGPEEESEGPEPAKRAGPLGQGALDAVQSLPLKSPFYDSSDNPYTRWLASTEGLQYS

SEQ ID NO: 89 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINNILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 90 - protein, 365 aa, Mus musculus
MLAVGAMEGPRQSAFLLSSPPLAALHSMAEMKTPLYPAAYPPLPTGPPSSSSSSSSSSSPSPPLGSHNPGGLKPPAAGGLSSLGSPPQQLSAATPHGINDILSRPSMPVASGAALPSASPSGSSSSSSSSASATSASAAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGTSENEEDDDDYNKPLDPNSDDEKITQLLKKHKSSGGSLLLHASEAEGSS

SEQ ID NO: 91 - protein, 346 aa, Homo sapiens
MGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 92 - protein, 349 aa, Mus musculus
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 93 - protein, 349 aa, Mus musculus; identical to SEQ ID NO: 92

SEQ ID NO: 94 - DNA, 1676 bp, Homo sapiens
acatcgattaactttttctcagaggcattcattttgtaatgggcaggtacttttcgcaagcatttgtacaggtttagggagtggaagctgaaggcgatctttcttttgatatagcgtttttctgcttttctttctgtttgcctctcccttgttgaatgtaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatctcgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccgcacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaggcacgccagtttcaccatttccgggaaacgaacccactgtgcttacagtgactgtcgtgtttacaaaaggcagccctttggtactactgctgcaaagtgcaaatactccaagcttcaagtgatatatgtatttattgtcattactgcctttggaagaaacaggggatcaaagttcctgttcaccttatgtattattttctatagactcttctattttaaaaaataaaaaaatacagtaaagtttaaaaaatacaccacgaatttggtgtggctgtattcagatcgtattaattatctgatcgggataacaaaatcacaagcaataattaggatctatgcaatttttaaactagtaatgggccaattaaaatatatataaatatatatttcaaccagcattttactacttgttacctcccatgctgaattat

SEQ ID NO: 95 - DNA, 1315 bp, Mus musculus
ctgcagaggacaggtagccccgggtcgtacggacagtaagtgcgcttcgaaggccgacctccaaacctcctgtccgtctgtcggtcctgcacactgcaagatgcctgcccctttggagacctgcatctctgatctcgactgctccagcagcaacagcagcagcgacctgtccagcttcctcaccgacgaggaggactgtgccaggctacagcccctagcctccacctcggggctgtccgtgccagcccggaggagcgctcccgccctctccggggcatcgaatgttcccggtgcccaggacgaagagcaggaacggcggaggcggcgaggtcgcgctcgggtgcggtccgaggctctgctgcactccctgcggaggagtcgtcgcgtcaaagccaacgatcgcgagcgcaaccgcatgcacaacctcaacgctgcgctggacgccttgcgcagcgtgctgccctcgttccccgacgacaccaagctcaccaagattgagacgctgcgcttcgcctacaactacatctgggccctggctgagacactgcgcctggcagatcaagggctccccgggggcagtgcccgggagcgcctcctgcctccgcagtgtgtcccctgtctgcccgggcccccgagcccggccagcgacactgagtcctggggttccggggccgctgcctccccctgcgccactgtggcatcaccactctctgaccccagtagtccctcggcttcagaagacttcacctatggcccgggcgatccccttttctcctttcctggcctgcccaaagacctgctccacacgacgccctgtttcatcccataccactaggcctttgtaaggcaacatcaatacattcttcctcccccagtctaagagcaataatagatggggaactggctgaagcctccgggggccacacttacccccaagtgaattctgggagctttaaaggggggagggggaatacctgaccacttgttaggttgctgcaccctcgctgaagctgccctcggtctatttctccacccccagcacggcctcccccccccccgcccgcccccagacggcctttcgtttttgttgcactttctgaacttcacaaaaccttctttgtgactggctcagaactgaccccagccaccacttcagtgtggtttggaaaagggacagatgagcccctgaagacgaggtgaaaagtcaattttacaatttgtagaactctaatgaagaaaaacgagcatgaaaattcggtttgagccggctgacaatacaatggcaaggcttaaaaaggagccacaaggagtgggc

SEQ ID NO: 96 - DNA, 816 bp, Homo sapiens
cagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctc

SEQ ID NO: 97 - DNA, 1179 bp, Mus musculus
gaattcgcggccgcgttttcaagcaccctctcggtgccagggcccaggaagggcatagagaaggaacctgaggactcatccaggggctgccctgcccctcacagcacagttgatggacccaacagccccgggtagcagtgtcagctccctgccgctgctcctggtccttgccctgggtcttgcaattctccactgtgtggtagcagatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttacctccagcaggaccgggggcagatcctggtggtctgcttgatagtggtcatggtggtgttcatcattttagtcatcggcgtctgcacctgctgtcatcctcttcggaaacatcgtaaaaaaaagaaggaagagaaaatggagactttggataaagataaaactcccataagtgaagatattcaagagaccaatattgcttaacggttataaagttatcacaagctggtggcaagctacaaaagacctgactcattcccagatggacaggacatgtctcaggaaaacagctagcagaaatgaatgtttaaatattgtatttactttttttatttgtaactgtgtgttgcttgttattgtttttaataacgatatattttttttgttacagcctagtagttgagaaaaaataacctggttaggtgatgacaaaaataagggacatttgaatataaactttgttgccaggattattaaataaataaaagaaaagtggaaaagaagttagatttttaagaactaattcaccaccacgcaatggtagtacatgcctttaatcccaggacttgggaggcagaggcaggcaaatctctgtgagttcaaggccagcctggtctacaaagaaagttccaaaatagccaagactacaacagaggaacactgtctcaaaaaacctaaccaaccaaccaaccaaacaagcaagcaaaaccctgtcaataataggcggccgcgaattc

SEQ ID NO: 98 - DNA, 5340 bp, Homo sapiens
ggatccctcgtggccagggttcccttcaaggtgcttagccaggtcaggaggccctagagaagcatggtttggattttctttcccagaccaaaaaagctccaagttggttctctcccagtttctaacttgcagttaaataaatcaggcaaggctggcctatgaggcagacaagtgtgaagaaggagaaggaggaggagaaggagaaggagaaagaagaagaaggaggagaagaagaagaagaagaagaagaagaagaggaggaggaggaggaggaggaggaagcagcagcagcagcagcagcttgaatggacagtggttccccttgcctagaaaatgggaccattatttcttttctaatctgacccccagactcaggacttcctctattttctgcattttggggtctcttgttttgccttgaaaaaaaatgttttctcccaaatcaaggagcagtagctggtgcaagggaaaatctagggctaggagtcttaagatatgacttctatgtggttctgatagaacttgctgggtgaccttgagagagtcactccccctctctgggccttgattttttcatctttaaagaaggcctcaaattcccattcttatgagaagaagacaagctcctagtgagtggtgacctaagggagcagctgcagcaaaatgctaacctgacagtcccagatggtccctttattggttctgaccctggtctcaggcttcatttccccacagcaagggaaggagcctgctcacagagcaccagctaagatcagcaggaccgcgccacacccccgcccagtcctagagcccccctctcgctggttcctgagcataccaccctcttccttggaggaaaatttgcccccaagcagcctaggcggtaagaggctatcactagggcagactcacagacctacctcatcccctcaccccaccctacagtctcgaagtcgggtcctgtcccctcctgcagtttccgggagactcaggatatctggacctgctagaaagagaagccttcctcgcctaaggagacttaaaccgggatacttaaacctcccgcctcggcgtcttcctccaggcacgaccgggtcaagagagagaagcggaagctgcaacccctcactctgagtgaccggaagcagaagaccacgggatgtcccaggcggggacaagaggaggggctggggaagaaaggagggatgatgagttcagagtccctttggaaaggtttccagagagcgctaccagggacaacccaaggggctggggaagtccctgccttgtgctctctgtgcgatgcccgagtgatgcagaggcagggggctggagcaggtgactgctggcagctgctgtctgtctgtgattggaccggaggactaaggggagaaaaagtttatcagcttctcccagtgcctgcacgctgtggtagttcaaaagacacgagggggaggggcacagcagctctgcttcccagcgccttgggagactgaagtgaaaggaacgcttgagcccaggagttcgagaccatcctgggcaacaaagcaagaccgcccctcaccccatacaaaataaaaatacaaataaattagccgggcacagtggcgcatgcctgtagtctcagctactgggaaggctgaagtgggaggatagcttgagcccaggagatcaaggctgcagtgagctgtgattgcaccactgcagtccagcctgggcgacagaaggagaccgttttttggttttgtttgttcgtttaaaaaaaaaaagaagcaagagctcactgtgaactcctggttccttcctcccctcctcacacttcccagaactcttcctgtcacggttcctggccagaacgctgggatactatctacaagctgtagtaggcttgtagtaatggaatgtccgcttgaggggtccccgcacagccaaccccggcctctggagtgggatctatgggggtggggttctaagcgcctctggggagtgtgaggtagcatctcagggtgtggcagaggctcggacacccccaaaaggtctgtgaatggaagggacataggcaggatctctctcagtgatgtcccctgtcttccaggatgaagagaggcagtgaaacaccaggagagcagggcgtcctttagaattcctggacccttctccaggctgctagtcaggacaatgagctcgtggttgtctttgccactatcttcctgtgcgatttcagacaagccacctccctcactaagcctaaatttccccatgtgtaacgtgcaggcattgtaccctagaggcatcaaagtcccctccaggacagatgctaaggaaagataggctaggagcaaagccgtctgaggtggcctgaccagagccacacgaggctcttctcactgggcgaggctctttgaggaaccgagagttgctgggacccagcccgccctcgagagagcaaacagagcggcgctcccctcccccgaccccggccctttgtccggaatccagctgtgctgcgggggaggagcgggctcgcgtggcgcggccccagggccccggcgctgattggccggtggcgcgggcagcagccgggcaggcacgctcctggcccgggcgaagcagataaagcgtgccaaggggcacacgacttgctgctcaggaaatccctgcggtctcaccgccgcgcctcgagagagagcgtgacagaggcctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggtaatatttggaggcctccgagggacgggcaggggaaagagggatcctctgacccagcgggggctgggaggatggctgtttttgttttttcccacctagcctcggaatcgcggactgcgccgtgacggactcaaacttacccttccctctgaccccgccgtaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccacctcttccgcctgcttgagcccaggcagtctggctttctcagattttctgtgaaaggacctgtctgtcgctgggctgtgggtgctaagggtaagggagagggagggagccgggagccgtagagggtggccgacggcggcggccctcaaaagcacttgttccttctgcttctccctggctgacccctggccggcccaggctccacgggggcggcaggctgggttcattccccggccctccgagccgcgccaacgcacgcaacccttgctgctgcccgcgcgaagtgggcattgcaaagtgcgctcattttaggcctcctctctgccaccaccccataatctcattcaaagaatactagaatggtagcactacccggccggagccgcccaccgtcttgggtcgccctaccctcactcaagtctgtctgcctctcagtctcttaccacccctcctccaatgtgattcaatccaatgtttggtctctcagcgcttactccccttgccttgctccaaagacgctgccgatctgctctactcccaatcaggtccgggatttcagggcgcctcactctgccttaaagccacgaaggcgaccctctgccttctcctcgtgcacttttcggagccattgccctcccggggcggaagaccaggctgtgaactgggaaagcgctagcccggccagggagcatctccccagcctccctgcgaactgcgcctgaaacgtgagctgcgctgcaggtgcctggagcaccgcgcatcttttttttttaaatctgtttgtaaattatatgatgccttttgaaatcaattttggtacagtaaaattatatggcccctcccctgttttacacatttgtatttattaatgagatttcacagcagggaaaagcctatattttggatattagattatttagggattgctggatgacatttaagccaataaaaaaaaatggaccttcaagaagccttggcaagatgactccattgtgtgttggggagaggagggccacagtcactacagctgaggaagagcacttctgtccaaagagagggatgacactctttctggaggtctgggctagagccagggcagattgggtttggagagctggaagtcttctaagtaattattggtccagctcccttttttctatatagggcaatgactcctcttatttcaaagagtggtttagaagaaagacaagcctccaactaggacaactgactctcacttgctggccctttccccaactccaccagcctagctttagagcaactgttggttgcacttggggaagggatacagtaataattcaattgcagagtcagagtcctcggaaacacggctgggctgggcatcctaggaattttcccaaggtgcttagaggcctagcaaatcccctgagcatattttactccccaggcactgaggtggctgtgtcgtgaactccttgaactgagcagccaggagcaaagaaggtggagcgtctggctggaatatccagcaacgccccctccctcatcacctggcagccttgattgaaaacttattaagaaactgttcaaggtttccagccacaccatgtctcttactggcaaggtggaataggactggtgcagcatgagcactgaaatctgtcccaggagtgccagtagagcaccactacatgacttcagggacccctaggacctcagagaatatggtctaagctgtaaggatcc

SEQ ID NO: 99 - DNA, 1861 bp, Mus musculus
ggatcccaaggtgatattgaacctggccaagcaatagtttctgagtagaaaggacttgagcagggaccgtctctggtcactctgtcctctttcccaggatggagtcagtctgtgaaacatggttgcacacacatttcctgacccaacccatagtggcggagagctggatagcactttgaactaatgggcgctcctcccagctgccagccaagaagacacttgactccttgatcgctggttcatttagacaagccgtttccctctctgagccaaaagaccccatgtgtaatactcaaagaagaggccttccttatatatatataggcacccccaaacctccttcatgctaccaagaaagggtctggacacatgccaaaaagaaagaggaaaaggcaaagctctccccagcggccggacgggactcttctggctgggcgaggctctttgaggaaccgagagttgctgggactgagcccgcgacgggggaggcgtggagtgggggaacaaacagagtgctgctcccctcccccgacccctgccctttgtccggaatccagctgtgctctgcgggtgggggttgtggggggaggagcgggctcgcgtggcgcagcccctgggccccctccgctgattggcccgtggtgcaggcagcagcccggcaggcacgctcctggccgggggcagagcagataaagcgtgccaggggacacacgacttgcatgcagctcagaaatccctctgggtctcatcactgcagcagtggtcgagtacctcctcggagcttttctacgacttccagacgcaatttactccaggcgagggcgcctgcagtttagcagaacttcagagggagcagagaggctcagctatccactgctgcttgacactgaccctatccactgctgcttgtcactgactgacctgctgctctctattcttttgagtcgggagaactaggtaacaattcggaaactccaaagggtggatgaggggcgcgcggggtgtgtgtgggggatactctggtcccccgtgcagtgacctctaagtcagaggctggcacacacacaccttccattttttcccaaccgcaggatggcgcctcatcccttggatgcgctcaccatccaagtgtccccagagacacaacaaccttttcccggagcctcggaccacgaagtgctcagttccaattccaccccacctagccccactctcatacctagggactgctccgaagcagaagtgggtgactgccgagggacctcgaggaagctccgcgcccgacgcggagggcgcaacaggcccaagagcgagttggcactcagcaaacagcgaagaagccggcgcaagaaggccaatgatcgggagcgcaatcgcatgcacaacctcaactcggcgctggatgcgctgcgcggtgtcctgcccaccttcccggatgacgccaaacttacaaagatcgagaccctgcgcttcgcccacaactacatctgggcactgactcagacgctgcgcatagcggaccacagcttctatggcccggagccccctgtgccctgtggagagctggggagccccggaggtggctccaacggggactggggctctatctactccccagtctcccaagcgggtaacctgagccccacggcctcattggaggaattccctggcctgcaggtgcccagctccccatcctatctgctcccgggagcactggtgttctcagacttcttgtgaagagacctgtctggctctgggtggtgggtgctagtggaaagggaggggaccagagccgtctggagtgggaggtagtggaggctctcaagcatctcgcctcttctggctttcactacttggatcc

SEQ ID NO: 100 - DNA, 2020 bp, Homo sapiens
caaagactcacccgtgagccagctctcaaagaaagcagcttgcgttgacagcctgggggcagcaaggatgcagtctcccaggagaggatgcactcggtggtgggaagccaggctggaggggcctgagtgaccctctccacaggcgggcagggcagtgggagaggtggtgtgtggatacctctgtctcacgcccagggatcagcagcatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaggcaggagtgggaaggagatggcatagagaagatctaataccatcctgcccattgtccttaccgtcctgcccatacagactgtggctccttcctccttcctgtgattgctccctcctgtgtggacgttgcctggccctgcctcgatgcctctctggcgcatcacctgattggaggggctggtaaagcaacacccacccacttctcacactggccttaagaggcctccactcagcagtaataaaagctgtttttattagcagtagttctgttgtccatcatgttttccctatgagcacccctatgcccactctaatattcaacaattatagacaatttgccctatcatttatttacatctatgtatctaccatctaatctatgcatgtatgtaggcaatacatgtatctaaacaatgtatttgtcaatgcatcaatttacctactctatgtatgcatctatatgtgtattatgtatgtatgtgtgcatgcgtgcgcgcacacacacacacacacattgatattatatcatggcattttattcctaaatcttccagcatgcatccccaaaaaacaagaaacttgtcttacataatcacaataatatatccacatctaagaaaatttactgtaacttcttaatctaagaaaattatgtatttttgtcatatgtattttgtcatatgtattttgtatttgcatatgtattttgtatttgcatatgtatttttgtcatagcagcaaacagagtgaaatgccatttttcatattcttaaaaaaaaaaa

SEQ ID NO: 101 - DNA, 1135 bp, Mus musculus
ccacgcgtccggtgaagcatgcagcaggacggactcagcagtgtgaatcagctagggggactctttgtgaatggccggccccttcctctggacaccaggcagcagattgtgcagctagcaataagagggatgcgaccctgtgacatttcacggagccttaaggtatctaatggctgtgtgagcaagatcctaggacgctactaccgcacaggtgtcttggaacccaagtgtattgggggaagcaaaccacgtctggccacacctgctgtggtggctcgaattgcccagctaaaggatgagtaccctgctctttttgcctgggagatccaacaccagctttgcactgaagggctttgtacccaggacaaggctcccagtgtgtcctctatcaatcgagtacttcgggcacttcaggaagaccagagcttgcactggactcaactcagatcaccagctgtgttggctccagttcttcccagtccccacagtaactgtggggctccccgaggtccccacccaggaaccagccacaggaatcggactatcttctccccgggacaagccgaggcactggagaaagagtttcagcgtgggcagtatccagattcagtggcccgtgggaagctggctgctgccacctctctgcctgaagacacggtgagggtttggttttctaacagaagagccaaatggcgcaggcaagagaagctgaaatgggaagcacagctgccaggtgcttcccaggacctgacagtaccaaaaaattctccagggatcatctctgcacagcagtcccccggcagtgtaccctcagctgccttgcctgtgctggaaccattgagtcctcccttctgtcagctatgctgtgggacagcaccaggcagatgttccagtgacacctcatcccaggcctatctccaaccctactgggactgccaatccctccttcctgtggcttcctcctcatatgtggaatttgcctggccctgcctcaccacccatcctgtgcatcatctgattggaggcccaggacaagtgccatcaacccattgctcaaactggccataagaggcctttatttgacagtaataaaaacctttttttagaaaaaaaaaaaaaaaaaaaaaaaaaaaaa

SEQ ID NO: 102 - DNA, 1399 bp, Homo sapiens
agggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaa

SEQ ID NO: 103 - DNA, 1469 bp, Mus musculus
ggtgagcagatgtgtgagatcttctattctagaagtggacgtatatcccagttctcagagccccgtattcgagccccgtgggatccggaggctgccaaccagctccagcatgcagaacagtcacagcggagtgaatcagcttggtggtgtctttgtcaacgggcggccactgccggactccacccggcagaagatcgtagagctagctcacagcggggcccggccgtgcgacatttcccgaattctgcagacccatgcagatgcaaaagtccaggtgctggacaatgaaaacgtatccaatggttgtgtgagtaaaattctgggcaggtattacgagactggctccatcagacccagggcaatcggagggagtaagccaagagtggcgactccagaagttgtaagcaaaatagcctagtataaacgggagtgcccttccatctttgcttgggaaatccgagacagattattatccgagggggtctgtaccaacgataacatacccagtgtgtcatcaataaacagagttcttcgcaacctggctagcgaaaagcaacagatgggcgcagacggcatgtatgataaactaaggatgttgaacgggcagaccggaagctggggcacacgccctggttggtatcccgggacttcagtaccagggcaacccacgcaagatggctgccagcaacaggaaggagggggagagaacaccaactccatcagttctaacggagaagactcggatgaagctcagatgcgacttcagctgaagcggaagctgcaaagaaatagaacatcttttacccaagagcagattgaggctctggagaaagagtttgagaggacccattatccagatgtgtttgcccgggaaagactagcagccaaaatagatctacctgaagcaagaatacaggtatggttttctaatcgaagggccaaatggagaagagaagagaaactgaggaaccagagaagacaggccagcaacactcctagtcacattcctatcagcagcagcttcagtaccagtgtctaccagccaatcccacagcccaccacacctgtctcctccttcacatcaggttccatgttgggccgaacagacaccgccctcaccaacacgtacagtgctttgccacccatgcccagcttcaccatggcaaacaacctgcctatgcaacccccagtccccagtcagacctcctcgtactcgtgcatgctgcccaccagcccgtcagtgaatgggcggagttatgatacctacacccctccgcacatgcaaacacacatgaacagtcagcccatgggcacctcggggaccacttcaacaggactcatttcacctggagtgtcagttcccgtccaagttcccgggagtgaacctgacatgtctcagtactggcctcgattacagtaaagagagaaggagagagcatgtgatcgagagaggaaattgtgttcactct

SEQ ID NO: 104 - DNA, 576 bp, Homo sapiens
tccctccccccactcccccctcccccgcccgccggggcaggggagcgccacgaattgaccaagtgaagctacaactttgcgacataaattttggggtctcgaaccatgtcgctgaccaacacaaagacggggttttcggtcaaggacatcttagacctgccggacaccaacgatgaggagggctctgtggccgaaggtccggaggaagagaacgaggggcccgagccagccaagagggccgggccgctggggcagggcgccctggacgcggtgcagagcctgcccctgaagaaccccttctacgacagcagcgacaacccgtacacgcgctggctggccagcaccgagggccttcagtactcccgtaagtagcaaaacttggctgccgaggccgtggtcccctccattcctgcagccgcagcccgggttggacgctgggagtgaaaggggaaggggccatgtaagcccggaccccctcactcggatccgtagaaagatttttaacacctgtataggatgtcctctgccctcctcttcaagcctccttagttccgggaaagaacttggtctccaaaa

SEQ ID NO: 105 - DNA, 1759 bp, Homo sapiens
tgcccacgtctcgccggagaggaaaccgcttaagggcgccggagcccttaacccgcgatgatcttcagtgccacttccccccccaaattctcacccacactatgtgagccccttgaaaggcgagcccctagcccccactcctacggatttccctctttaccctgggaggtcccgacgtcttcgtcaggcgtagaggaaggcaggggtcatggcaaaggcagcggggctgggctgccaggcgcggaggtccagggtcgcacggaggatccagggtgctccgagtctggtgcaggctgcgcgcggcctccagacgcctgacgcgcttctctctccccctccccccagtgcacggtctggctgccggggcgccccctcaggactcaagctccaagtccccggagccctcggccgacgagtcaccggacaatgacaaggagaccccgggcggcgggggggacgccggcaagaagcgaaagcggcgagtgcttttctccaaggcgcagacctacgagctggagcggcgctttcggcagcagcggtacctgtcggcgcccgagcgcgaacacctggccagcctcatccgcctcacgcccacgcaggtcaagatctggttccagaaccaccgctacaagatgaagcgcgcccgggccgagaaaggtatggaggtgacgcccctgccctcgccgcgccgggtggccgtgcccgtcttggtcagggacggcaaaccatgtcacgcgctcaaagcccaggacctggcagccgccaccttccaggcgggcattcccttttctgcctacagcgcgcagtcgctgcagcacatgcagtacaacgcccagtacagctcggccagcaccccccagtacccgacagcacaccccctggtccaggcccagcagtggacttggtgagcgccgccccaacgagactcgcggccccaggcccaggccccaccccggcggcggtggcggcgaggaggcctcggtccttatggtggttattattattattataattattattatggagtcgagttgactctcggctccactagggaggcgccgggaggttgcctgcgtctccttggagtggcagattccacccacccagctctgcccatgcctctccttctgaaccttgggagagggctgaactctacgccgtgtttacagaatgtttgcgcagcttcgcttctttgcctctccccggggggaccaaaccgtcccagcgttaatgtcgtcacttgaaaacgagaaaaagaccgaccccccacccctgctttcgtgcattttgtaaaatatgtttgtgtgagtagcgatattgtcagccgtcttctaaagcaagtggagaacactttaaaaatacagagaatttcttcctttttttaaaaaaaaataagaaaatgctaaatatttatggccatgtaaacgttctgacaactggtggcagatttcgcttttcgttgtaaatatcggtggtgattgttgccaaaatgaccttcaggaccggcctgtttcccgtctgggtccaactcctttctttgtggcttgtttgggtttgttttttgttttgtttttgtttttgcgttttcccctgctttcttcctttctctttttattttattgtgcaaacatttctcaaatatggaaaagaaaaccctgtaggcagggagccctctgccctgtcctccgggccttcagccccgaacttggagctcagctattcggcgcggttccccaacagcgccgggcgcagaaagctt

SEQ ID NO: 106 - DNA, 3103 bp, Mus musculus
ctgcagtagggtaacatttttcatctcttattttctgtggccaggaggaagatgccattcagagaaccccaggtgttttgaagatgagaaggaaggtaggaggcctggctcagtgcttattaaccacagagagagctgggttcacttcaagaaagaatcaaataatggccaggagagacaatgactcttaatgaattcatgtgagggaagtgtgaggtgaccagtttggggacatgcagtctgcaaactgctttctgaaggagaaaagcaagacaattgttttctattatggtccaatagtacaatatatccttgctttcctggggcacatgcggctggctgggtttcacatacagctgctggtgtggcttcctaggagggccttagctgcctttactttaaatacagcctgggcttgagaaagcccagtccatgaggaaggaggagtctcagttctctctccaggtgagctaccccttcctaggtttcctgtcctgattcccacctacccacccacccaccccaattaatttctccctagagggtctgggacccccccccacttactccacctaggtgagagagagcaaacccaggtttcctggatcagacttagtgtcatggactttctggaagaaagagagagagagagagagagagagagagagagagagagagagagagagagagagaagagaagagaagagaagagaagaagaaaagaaaagaaaagaaaagaaaagaaaagaaaagaaaagaaaagaaaagaaaagaaaagaaaagaaaagaaaagaaaagaaaagaaaagaaaagaaaagaaaagaaaagaaaagaaaagagcctcccagacttgagggatggggaaggggacaagagagaggaaacagaagcggggtctgaagaggggtggggagtgaggttgactaatcctgcacaggtggcaccttggcacaaatacttccgtggctgcagagagctgaacacctttcccgggaagacttacacccctcaatctaggctgggtagtgcaccaggggaggagactgaagcagaggcccagaggtctgtgacctcctacgaagtgctatctgccttggactcgcatatctagagtaaatgcgctttcacacataaataacgtgccacatctggccctttggttttatggcgtcttagcgaccaagcaatttatagatggcgaccttgttaaccagcagccagggctcgccaggagctacaccgcgccgggcactgatgggcacagaggaggggggtcgagtgcaaggaagactgtgggccctggtctccagctcagagggacgccacggagaaatcccacctcggattgggggagaagggggccacgacagggtggaaggtggaaaccccctccctctcaagccggccatgtggcagctgaaagagccatcgaagcccaagtgtgtttgcgctcatacccaatttattaccgctgaacatatggccaatattttgactcacgtcagtcgggctagaaaaacaaacagagcgctgcgcgggggagcggcccctccgcggaggctgcggctgccgcggggccgggagcgggcgagtgagcgcggctggagccccagtccgaagctgggaggagggcgctcgcccagcagcgccacaacccgggctccccagcggcaggccggagtacccgaggcccggaacagcaagctagccgaggcggaaccgcgccgccgcactccaggtgagcctcctgttcgcgcacgtcctgcatgcatgcttgctcacggggcgcccgcagctgcgtctagcggccaccgggctgcagaaggagccgggagcccagagctctcccgccctcccactgccaggacacactgtctgttctcctggggctgaccggcgctggggtcggtctttgctaccgctgggatgcgaccacatcccgggcagggacctcgctacgcctagaggaccaagcctctgaacagctcggcgaccctctcgtccatctctagatggactctgtcccagagtcacggagtcgggaagcagcaggtgggcagatccaggcatcttaagccctccaaggacttggctggtcaccaggggaaggaggcctcgagaagggacagcctcctccctctggtcgcatttgtcgttgagaaaagttttacctgccacagactccaggtgttcccgcagccacacctgacagcacgtggggctgccttgggggctgggggggggtagctgggccgctggagttccccccccccaccagctccgtgggcaggagcggcgcccactgccaccaccgcccacgtcgagccctgctgaccctcgagccccgcccgggaggcctgcggcagggggaagggcgcagcgggagggggcgtcccgggaggtggaggactagataaaggcgggtgttgaaacgccgcggagctgtccccgcagcgcggggagtgggaggcggaacgggcgcgtccagcgccttgtcagcctcctccccaccgcccccgcccccggggctcttcgactttgctggctgcagtgaggaaggacgcgccgcggcccccacctttgaggggtgagtctcctggtgcgcgccgctggtgactcacgattcagcttggtaagcagaggccagaataccaaggaatttggagatggcgcctaatgagaaaggaaggttccttctggggcggaactggcacgcgagtggctccgggagccgtaaggagctggagggtgagcggcgggaatcagactcaaggacccacagggctgggggctggagtcgtagacggtgatcttcggggaggcgacgaaaacctgctgcttgaaacattagtgtctcctgggcctcaccgggctctggacagttctcatttgtgacagtagagatcgccagaggatcagaaatgaaccttggtgggccctgttaaagtcagggtgtcttctggaagacccctccactgatcctatcataccctcccctccccagctcgcagactcacaaacaggcctttctatgcctctcgctacttctccactttggtggcacttggaatttggtatggagaggggacgttgcttcctgaaa

SEQ ID NO: 107 - DNA, 1834 bp, Mus musculus
  modified_base (234): n = a, c, g or t/u
ggtacccaggctcttcccttcgccagctggaaataagctgaggccacaggcgcgtagggccatggtccgaaccctgccactgctagagccgcagtccgcccctcccttctctagaccgcccgcagagcagaaagtggagggccagtcctcttcctccgctagaaactgggggctggggggggggggggaggtaaatgagtctttagcaaataaaaggcggccggagggggtggncctcagtggtagctctggcatgtccaagcctattcctctcctgggtttccagctctttcgcggttaactagaatcaattacttgacttgtcatttctagtacctcatcccttaagcttcaaccaaaaatattgacctaggaggcttacagaaaagttgggctcttgccatttgaactggatccttgtttagggaaccaccagcgatgggaagggagagatttcagcggctctgccttctccccctcccccttttccaaaggcaccacaaatcgctaattttctggctagttggggggctgaaggagggggcttgtgcacggggcgtggagaggtagaaacctgtgaatgttaaaaaggattctttgccccctcctttctgttttgttttctttctcaccccaaaccccccccccttaagatgcaatttgttaaaacggctcttttcaagtgtgtggactcgagagcgacgcgggtggtcctttgtatgtaaatactgagggagaaaaaaagctctccccatctttgcaattaattgacacgttacacctctcatcttgctctagagggctgttggctgggagcgcagagctccccaaaacccacaatttcacatctgcaaatactgtcttcatccacttgactcccaagacccgcccacacgtggccaacctttgcggttttaatgtctcttcccccctttttttcacccctctctcgctccctcgaccccctccctcttttcctccctccctttttctccccctcccccctccccaggttcgtgagtggagcccagccttatatggactgatcgctcaggcaatggcccattttttcctcggccaccagccgccaccgcgcgcggagcggccgcggagccggagctgacggcaccttggcacctctcctggagttacaaactgaggccgcgcggcgctgggcgcaggccccagtcacagcctacatttctgcgtgctttccgagaagagagaggcaccgggtgggctttattttttttcccctttcccttttccccccacagtgtcctctcattttaaataataaattatcccaataattaaaaccccatcccccatccctccccccattcctttcctttaaacccccctcccccgcccgctggggctggggagagccacgaattgaccaagtgaggctacaactttgtttggcataaattgcggggtccggaaccatgtcgctgaccaacacaaaagacggggttttcaaggtcaaggacatcttggaccttccggacaccaacgatgaagacggctcggtggccgaagggccagaggaggagagcgaagggccggagcccgccaagagggccgggccgctggggcagggcgccctggacgctgtgcagagcctgccccttaagagccctttctacgacagcagcgacaacccctacactcgctggctggccagcaccgagggcctccaatactcccgtaagtagcgaaacttggccgcaacggctgtggctgcctccattgctgaggcagtaacccgggtggacgctgggagttttggggaagaagccatttaatgtccaagtcccatccgggatcc

SEQ ID NO: 108 - DNA, 682 bp, Homo sapiens
cgtgggatgttagcggtgggggcaatggagggcacccggcagagcgcattcctgctcagcagccctcccctggccgccctgcacagcatggccgagatgaagaccccgctgtaccctgccgcgtatcccccgctgcctgccggccccccctcctcctcgtcctcgtcgtcgtcctcctcgtcgccctccccgcctctgggcacccacaacccaggcggcctgaagcccccggccacgggggggctctcatccctcggcagccccccgcagcagctctcggccgccaccccacacggcatcaacaatatcctgagccggccctccatgcccgtggcctcgggggccgccctgccctccgcctcgccctccggttcctcctcctcctcttcctcgtccgcctctgcctcctccgcctctgccgccgccgcggctgctgccgcggccgcagccgccgcctcatccccggcggggctgctggccggactgccacgctttagcagcctgagcccgccgccgccgccgcccgggctctacttcagccccagcgccgcggccgtggccgccgtgggccggtaccccaagccgctggctgagctgcctggccggacgcccatcttctggcccggagtgatgcagagcccgccctggagggacgcacgcctggcctgtacccctcgtgagt

SEQ ID NO: 109 - DNA, 185 bp, Homo sapiens
tcacagatcaaggatccattttgttggacaaagacgggaagagaaaacacacgagacccactttttccggacagcagatcttcgccctggagaagactttcgaacaaacaaaatacttggcggggcccgagagggctcgtttggcctattcgttggggatgacagagagtcaggtcaaggtgagt

SEQ ID NO: 110 - DNA, 273 bp, Homo sapiens
cctcaggtctggttccagaaccgccggaccaagtggaggaagaagcacgctgccgagatggccacggccaagaagaagcaggactcggagacagagcgcctcaagggggcctcggagaacgaggaagaggacgacgactacaataagcctctggatcccaactcggacgacgagaaaatcacgcagctgttgaagaagcacaagtccagcagcggcggcggcggcggcctcctactgcacgcgtccgagccggagagctcatcctgaacgccg

SEQ ID NO: 111 - DNA, 1815 bp, Mus musculus
ccgccgggagagcggagcgtccgagcgagatcagaggcgcgcaccgggcggaacgccgcccgctttgaagctcccccaggcgagcgagccggcccccgccctcctacatcaaagcgaacgctccgcgcctcccaaccttgttgcaaactctctgggtcggctgcggggtacgtcttgctgatttcccgcgggggtggagaagatgagaagcagagcgctctgagccgggaacgagggaccagcgcctgggatcgaatccgggactcccgaagccgaggaagcgctgagcccgcccgcgcccccgcagccctcgcccctgccgcctcccgcggggcgtttggacatttttgctgcgcagctcccggagcccgcggccgatccacacttcgcttgcgcgcgcccccggcacctcgggttctcccgagccccggcggggccaccgacctgcgtggctgcgggttcgggtctggctgtgggatgttagctgtgggggcgatggagggccctcggcagagcgcgttcctgctcagcagcccgcccctggccgccctgcacagtatggccgagatgaagaccccgctctaccccgccgcttatcccccgctgcccaccgggcccccctcctcctcgtcctcgtcctcctcgtcctcgtcgccctccccacctttgggctcacataacccgggcggcttgaagcccccggccgcggggggcctctcgtccctgggcagtcccccgcagcagctttcggcggccaccccacacggcatcaacgacatcctgagccggccctctatgccggtggcctcgggggccgccctgccctccgcctcgccctccgggtcttcctcctcctcctcctcgtccgcctccgccacctcggcctctgcggcggccgccgccgccgctgctgctgccgccgctgccgcctcgtcgcccgctgggctgctggccggcctgccccgcttcagcagcctgagccctccgccaccgccgcccgggctctactttagccccagcgccgcggctgtggccgccgtgggccggtaccccaagcccctggccgagctgcccggtcggacgcccatcttctggcccggagtgatgcagagtccgccgtggagggacgcgcgccttgcctgtaccccccatcaaggatccattttgttggacaaagatgggaagagaaaacacaccagacccacgttctctggacagcaaatcttcgccctggagaagactttcgaacaaacgaagtacttggcaggaccagagagagcacgcttggcctattctctggggatgacggagagtcaggtcaaggtctggttccagaaccgcaggaccaagtggagaaagaagcacgcagccgagatggccacggccaagaagaagcaggactcggagaccgagaggctcaaggggacttcggagaatgaggaggatgacgacgattacaacaaacctctggacccgaactctgacgacgagaaaatcactcagctgctgaaaaagcacaaatcgagcggtggcagcctcctgctgcacgcgtcggaggccgagggctcgtcctgagcgcgaccagcaccgcggggatcgcgaccgcgtcccacagccggttcccccggcccccagtatcctggctgctcgccgggcctttactattttttaagatgtacatatctatttttttaacctagaaattgtggcgggaagggtgcgggtcggtagcacggtgcgctgatgaggagaaaaggagcccgccaagtgcactgctcaaaaaaccaaaaaccaaaaaaaaaaaaaaaaaaaaaa

SEQ ID NO: 112 - DNA, 2397 bp, Homo sapiens
cccccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagat

SEQ ID NO: 113 - DNA, 1815 bp, Mus musculus; identical to SEQ ID NO: 111

SEQ ID NO: 114 - DNA, 942 bp, Homo sapiens
cccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccggg

SEQ ID NO: 115 - DNA, 1463 bp, Mus musculus
aaaattgaaacaagtgcaggtgttcgcgggcacctaagcctccttcttaaggcagtcctccaggccaatgatggctccagggtaaaccacgtggggtgccccagagcctatggcacggcggccggcttgtccccagccagcctctggttccccaggagagcagtggagaactgtcaaagcgatctggggtggcgtagagagtccgcgagccacccagcgcctaaggcctggcttgtagctccgacccggggctgctggccccaagtgccggctgccaccatgaacagtgaggagcagtactacgcggccacacagctctacaaggacccgtgcgcattccagaggggcccggtgccagagttcagcgctaacccccctgcgtgcctgtacatgggccgccagcccccacctccgccgccaccccagtttacaagctcgctgggatcactggagcagggaagtcctccggacatctccccatacgaagtgcccccgctcgcctccgacgacccggctggcgctcacctccaccaccaccttccagctcagctcgggctcgcccatccacctcccggacctttcccgaatggaaccgagcctgggggcctggaagagcccaaccgcgtccagctccctttcccgtggatgaaatccaccaaagctcacgcgtggaaaggccagtgggcaggaggtgcttacacagcggaacccgaggaaaacaagaggacccgtactgcctacacccgggcgcagctgctggagctggagaaggaattcttatttaacaaatacatctcccggccccgccgggtggagctggcagtgatgttgaacttgaccgagagacacatcaaaatctggttccaaaaccgtcgcatgaagtggaaaaaagaggaagataagaaacgtagtagcgggaccccgagtgggggcggtgggggcgaagagccggagcaagattgtgcggtgacctcgggcgaggagctgctggcagtgccaccgctgccacctcccggaggtgccgtgcccccaggcgtcccagctgcagtccgggagggcctactgccttcgggccttagcgtgtcgccacagccctccagcatcgcgccactgcgaccgcaggaaccccggtgaggacagcagtctgagggtgagcgggtctgggacccagagtgtggacgtgggagcgggcagctggataagggaacttaacctaggcgtcgcacaagaagaaaattcttgagggcacgagagccagttggatagccggagagatgctgcgagcttctggaaaaacagccctgagcttctgaaaactttgaggctgcttctgatgccaagctaatggccagatctgcctctgaggactctttcctgggaccaatttagacaacctgggctccaaactgaggacaataaaaagggtacaaacttgagcgttccaatacggaccagcaggcgag

SEQ ID NO: 116 - DNA, 20 bp, Artificial Sequence
  Description of Artificial Sequence  Synthetic Primer
gtactgcctacacccgggcg

SEQ ID NO: 117 - DNA, 21 bp, Artificial Sequence
  Description of Artificial Sequence  Synthetic Primer
aggcagcctgcacctgaggag

SEQ ID NO: 118 - DNA, 20 bp, Artificial Sequence
  Description of Artificial Sequence  Synthetic Primer
gtactgcctacacccgggcg

SEQ ID NO: 119 - DNA, 20 bp, Artificial Sequence
  Description of Artificial Sequence  Synthetic Primer
tgcaacttcccaaggcagga

SEQ ID NO: 120 - DNA, 21 bp, Artificial Sequence
  Description of Artificial Sequence  Synthetic Primer
gaaagccccctaactgactgc

SEQ ID NO: 121 - DNA, 21 bp, Artificial Sequence
  Description of Artificial Sequence  Synthetic Primer
aggcagcctgcacctgaggag

SEQ ID NO: 122 - DNA, 20 bp, Artificial Sequence
  Description of Artificial Sequence  Synthetic Primer
atggacccaacagccccggg

SEQ ID NO: 123 - DNA, 21 bp, Artificial Sequence
  Description of Artificial Sequence  Synthetic Primer
aggcagcctgcacctgaggag

SEQ ID NO: 124 - DNA, 22 bp, Artificial Sequence
  Description of Artificial Sequence  Synthetic Primer
atggccctgttggtgcacttcc

SEQ ID NO: 125 - DNA, 25 bp, Artificial Sequence
  Description of Artificial Sequence  Synthetic Primer
ttagttgcagtagttctccagctgg

SEQ ID NO: 126 - DNA, 20 bp, Artificial Sequence
  Description of Artificial Sequence  Synthetic Primer
atggccctgtggatgcgctt

SEQ ID NO: 127 - DNA, 25 bp, Artificial Sequence
  Description of Artificial Sequence  Synthetic Primer
ctagttgcagtagttctccagctgg

SEQ ID NO: 128 - DNA, 25 bp, Artificial Sequence
  Description of Artificial Sequence  Synthetic Primer
atgaagaccatttactttgtggctg

SEQ ID NO: 129 - DNA, 20 bp, Artificial Sequence
  Description of Artificial Sequence  Synthetic Primer
cggcctttcaccagccacgc

SEQ ID NO: 130 - DNA, 20 bp, Artificial Sequence
  Description of Artificial Sequence  Synthetic Primer
atgctgtcctgccgtctcca

SEQ ID NO: 131 - DNA, 30 bp, Artificial Sequence
  Description of Artificial Sequence  Synthetic Primer
ctaacaggatgtgaatgtcttccagaagaa

SEQ ID NO: 132 - DNA, 20 bp, Artificial Sequence
  Description of Artificial Sequence  Synthetic Primer
atggccgtcgcatactgctg

SEQ ID NO: 133 - DNA, 19 bp, Artificial Sequence
  Description of Artificial Sequence  Synthetic Primer
tcgctccagggcgcagagc

SEQ ID NO: 134 - DNA, 20 bp, Artificial Sequence
  Description of Artificial Sequence  Synthetic Primer
atggacccaacagccccggg

SEQ ID NO: 135 - DNA, 25 bp, Artificial Sequence
  Description of Artificial Sequence  Synthetic Primer
agctgttttcctgagacatgtcctg

SEQ ID NO: 136 - DNA, 22 bp, Artificial Sequence
  Description of Artificial Sequence  Synthetic Primer
ggagctgctgttgctttccctg

SEQ ID NO: 137 - DNA, 22 bp, Artificial Sequence
  Description of Artificial Sequence  Synthetic Primer
agcaggtctgggttgttcacac

SEQ ID NO: 138 - DNA, 23 bp, Artificial Sequence
  Description of Artificial Sequence  Synthetic Primer
ggcccagagagttacctgttgcc

SEQ ID NO: 139 - DNA, 26 bp, Artificial Sequence
  Description of Artificial Sequence  Synthetic Primer
gcgccatcctggctctgtcatccagc

SEQ ID NO: 140 - DNA, 18 bp, Artificial Sequence
  Description of Artificial Sequence  Synthetic Primer
gcggaagtccttggctgc

SEQ ID NO: 141 - DNA, 20 bp, Artificial Sequence
  Description of Artificial Sequence  Synthetic Primer
accagaatcaacaactgggc

SEQ ID NO: 142 - DNA, 25 bp, Artificial Sequence
  Description of Artificial Sequence  Synthetic Primer
atggagcaaagaggttggactctgc

SEQ ID NO: 143 - DNA, 25 bp, Artificial Sequence
  Description of Artificial Sequence  Synthetic Primer
gattccacattggatcattgaagct

SEQ ID NO: 144 - DNA, 20 bp, Artificial Sequence
  Description of Artificial Sequence  Synthetic Primer
atggagggcggttgtggatc

SEQ ID NO: 145 - DNA, 25 bp, Artificial Sequence
  Description of Artificial Sequence  Synthetic Primer
caggtaccattgctttgtaaagaga

SEQ ID NO: 146 - DNA, 20 bp, Artificial Sequence
  Description of Artificial Sequence  Synthetic Primer
atgctgtcccgaaaggccat

SEQ ID NO: 147 - DNA, 25 bp, Artificial Sequence
  Description of Artificial Sequence  Synthetic Primer
ggtcacctggacctcgatggagaaa

SEQ ID NO: 148 - DNA, 19 bp, Artificial Sequence
  Description of Artificial Sequence  Synthetic Primer
atgcccttggccttctgcg

SEQ ID NO: 149 - DNA, 26 bp, Artificial Sequence
  Description of Artificial Sequence  Synthetic Primer
gtgatgaaggccaaggtccagtagat

SEQ ID NO: 150 - DNA, 27 bp, Artificial Sequence
  Description of Artificial Sequence  Synthetic Primer
atgaacagtgaggagcagtactacgcg

SEQ ID NO: 151 - DNA, 20 bp, Artificial Sequence
  Description of Artificial Sequence  Synthetic Primer
ggagcccaggttgtctaaat

SEQ ID NO: 152 - DNA, 20 bp, Artificial Sequence
  Description of Artificial Sequence  Synthetic Primer
gcgcaacaggcccaagagcg

SEQ ID NO: 153 - DNA, 20 bp, Artificial Sequence
  Description of Artificial Sequence  Synthetic Primer
tcacaagaagtctgagaaca

SEQ ID NO: 154 - DNA, 21 bp, Artificial Sequence
  Description of Artificial Sequence  Synthetic Primer
gaaagccccctaactgactgc

SEQ ID NO: 155 - DNA, 25 bp, Artificial Sequence
  Description of Artificial Sequence  Synthetic Primer
gcactttgcagcaatcttagcaaaa

SEQ ID NO: 156 - DNA, 24 bp, Artificial Sequence
  Description of Artificial Sequence  Synthetic Primer
ggccgtgagcaagatcctaggacg

SEQ ID NO: 157 - DNA, 24 bp, Artificial Sequence
  Description of Artificial Sequence  Synthetic Primer
gcgcgagaggtggcagcagccagc

SEQ ID NO: 158 - DNA, 20 bp, Artificial Sequence
  Description of Artificial Sequence  Synthetic Primer
atgcagaacagtcacagcgg

SEQ ID NO: 159 - DNA, 20 bp, Artificial Sequence
  Description of Artificial Sequence  Synthetic Primer
tcgctagccaggttgcgaag

SEQ ID NO: 160 - DNA, 20 bp, Artificial Sequence
  Description of Artificial Sequence  Synthetic Primer
atgtcgctgaccaacacaaa

SEQ ID NO: 161 - DNA, 20 bp, Artificial Sequence
  Description of Artificial Sequence  Synthetic Primer
tccttgtcattgtccggtga

SEQ ID NO: 162 - DNA, 24 bp, Artificial Sequence
  Description of Artificial Sequence  Synthetic Primer
ggccgagtgatgcagagtccgccg

SEQ ID NO: 163 - DNA, 24 bp, Artificial Sequence
  Description of Artificial Sequence  Synthetic Primer
gcgccctcctcattctccgaagtc

SEQ ID NO: 164 - DNA, 20 bp, Artificial Sequence
  Description of Artificial Sequence  Synthetic Primer
atgggagacatgggcgatcc

SEQ ID NO: 165 - DNA, 20 bp, Artificial Sequence
  Description of Artificial Sequence  Synthetic Primer
cgtggtctgcacggcagaaa

SEQ ID NO: 166 - DNA, 21 bp, Artificial Sequence
  Description of Artificial Sequence  Synthetic Primer
atgaagaccaaaaaccggccc

SEQ ID NO: 167 - DNA, 22 bp, Artificial Sequence
  Description of Artificial Sequence  Synthetic Primer
ctagctcccctctctgaagctg

SEQ ID NO: 168 - DNA, 22 bp, Artificial Sequence
  Description of Artificial Sequence  Synthetic Primer
atggatgacgatatcgctgcgc

SEQ ID NO: 169 - DNA, 18 bp, Artificial Sequence
  Description of Artificial Sequence  Synthetic Primer
tctgtcaggtcccggcca

SEQ ID NO: 170 - DNA, 1676 bp, Homo sapiens; identical to SEQ ID NO: 94

SEQ ID NO: 171 - DNA, 591 bp, Mus musculus
actacgcagcaccgaggtacagacacgccagcatgaagcactgcgtttaacttttcctggaggcatccattttgcagtggactcctgtgtatttctatttgtgtgcatttctgtaggattagggagagggagctgaaggcttatccagcttttaaatatagcgggtggatttccccccctttcttcttctgcttgcctctctccctgttcaatacaggaagtggaaacatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctg

SEQ ID NO: 172 - DNA, 5340 bp, Homo sapiens; identical to SEQ ID NO: 98

SEQ ID NO: 173 - DNA, 1861 bp, Mus musculus; identical to SEQ ID NO: 99

SEQ ID NO: 174 - DNA, 1399 bp, Homo sapiens; identical to SEQ ID NO: 102

SEQ ID NO: 175 - DNA, 1469 bp, Mus musculus; identical to SEQ ID NO: 103

SEQ ID NO: 176 - DNA, 2020 bp, Homo sapiens; identical to SEQ ID NO: 100

SEQ ID NO: 177 - DNA, 1135 bp, Mus musculus; identical to SEQ ID NO: 101

SEQ ID NO: 178 - DNA, 576 bp, Homo sapiens; identical to SEQ ID NO: 104

SEQ ID NO: 179 - DNA, 1759 bp, Homo sapiens; identical to SEQ ID NO: 105

SEQ ID NO: 180 - DNA, 3103 bp, Mus musculus; identical to SEQ ID NO: 106

SEQ ID NO: 181 - DNA, 1834 bp, Mus musculus; identical to SEQ ID NO: 107
  modified_base (234): n = a, c, g or t/u

SEQ ID NO: 182 - DNA, 682 bp, Homo sapiens; identical to SEQ ID NO: 108

SEQ ID NO: 183 - DNA, 185 bp, Homo sapiens; identical to SEQ ID NO: 109

SEQ ID NO: 184 - DNA, 273 bp, Homo sapiens; identical to SEQ ID NO: 110

SEQ ID NO: 185 - DNA, 1815 bp, Mus musculus; identical to SEQ ID NO: 111

SEQ ID NO: 186 - DNA, 2397 bp, Homo sapiens; identical to SEQ ID NO: 112

SEQ ID NO: 187 - DNA, 2168 bp, Homo sapiens
agataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaaaaaaaaaaaaaaaa

SEQ ID NO: 188 - DNA, 816 bp, Homo sapiens; identical to SEQ ID NO: 96

SEQ ID NO: 189 - DNA, 1179 bp, Mus musculus; identical to SEQ ID NO: 97

SEQ ID NO: 190 - DNA, 942 bp, Homo sapiens; identical to SEQ ID NO: 114

SEQ ID NO: 191 - DNA, 1463 bp, Mus musculus; identical to SEQ ID NO: 115

SEQ ID NO: 192 - DNA, 1513 bp, Mus musculus
gaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattagaggcacgtcagtttcactattcccgggaaacgaatccactgtgcgtacagtgactgtcctgtttacagaaggcagcccttttgctaagattgctgcaaagtgcaaatactcaaagcttcaagtgatatatgtatttattgtcgttactgcctttggaagaaacaggggatcaaagttcctgttcaccttatgtattgttttctatagctcttctattttaaaaataataatacagtaaagtaaaaaagaaaatgtgtaccacgaatttcgtgtagctgtattcagatcgtattaattatctgatcgggataaaaaaaatcacaagcaataattaggatctatgcaatttttaaactagtaatgggccaattaaaatatatataaatatatatttttcaaccagcattttactacctgtgacctttcccatgctgaattattttgttgtgattttgtacagaatttttaatgactttttataacgtggatttcctattttaaaaccatgcagcttcatcaatttttatacatatcagaaaagtagaattatatctaatttatacaaaataatttaactaatttaaaccagcagaaaagtgcttagaaagttattgcgttgccttagcacttctttcttctctaattgtaaaaaaga

SEQ ID NO: 193 - DNA, 1218 bp, Mus musculus
tgtttcccccagttttggcaaccccgggggccactatttgccacctagccacagcaccagcatcctctctgtgggctattcaccaattgtccaaccaccatttcactgtggacattactccctcttacagatatgggagacatgggcgatccaccaaaaaaaaaacgtctgatttccctgtgtgttggttgcggcaatcaaattcacgaccagtatattctgagggtttctccggatttggagtggcatgcagcatgtttgaaatgtgcggagtgtaatcagtatttggacgaaagctgtacgtgctttgttagggatgggaaaacctactgtaaaagagattatatcaggttgtacgggatcaaatgcgccaagtgcagcataggcttcagcaagaacgacttcgtgatgcgtgcccgctctaaggtgtaccacatcgagtgtttccgctgtgtagcctgcagccgacagctcatcccgggagacgaattcgccctgcgggaggatgggcttttctgccgtgcagaccacgatgtggtggagagagccagcctgggagctggagaccctctcagtcccttgcatccagcgcggcctctgcaaatggcagccgaacccatctcggctaggcagccagctctgcggccgcacgtccacaagcagccggagaagaccacccgagtgcggactgtgctcaacgagaagcagctgcacaccttgcggacctgctatgccgccaaccctcggccagatgcgctcatgaaggagcaactagtggagatgacgggcctcagtcccagagtcatccgagtgtggtttcaaaacaagcggtgcaaggacaagaaacgcagcatcatgatgaagcagctccagcagcagcaacccaacgacaaaactaatatccaggggatgacaggaactcccatggtggctgctagtccggagagacatgatggtggtttacaggctaacccagtagaggtgcaaagttaccagccgccctggaaagtactgagtgacttcgccttgcaaagcgacatagatcagcctgcttttcagcaactggtcaatttttcagaaggaggaccaggctctaattctactggcagtgaagtagcatcgatgtcctcgcagctcccagatacacccaacagcatggtagccagtcctattgaggcatgaggaacattcattcagatgttttgttttgttttgttt

SEQ ID NO: 194 - DNA, 1466 bp, Mus musculus
aaaattgaaacaagtgcaggtgttcgcgggcacctaagcctccttcttaaggcagtcctccaggccaatgatggctccagggtaaaccacgtggggtgccccagagcctatggcacggcggccggcttgtccccagccagcctctggttccccaggagagcagtggagaactgtcaaagcgatctggggtggcgtagagagtccgcgagccacccagcgcctaaggcctggcttgtagctccgacccggggctgctggcccccaagtgccggctgccaccatgaacagtgaggagcagtactacgcggccacacagctctacaaggacccgtgcgcattccagaggggcccggtgccagagttcagcgctaacccccctgcgtgcctgtacatgggccgccagcccccacctccgccgccaccccagtttacaagctcgctgggatcactggagcagggaagtcctccggacatctccccatacgaagtgcccccgctcgcctccgacgacccggctggcgctcacctccaccaccaccttccagctcagctcgggctcgcccatccacctcccggacctttcccgaatggaaccgagcctgggggcctggaagagcccaaccgcgtccagctccctttcccgtggatgaaatccaccaaagctcacgcgtggaaaggccagtgggcaggaggtgcttacacagcggaacccgaggaaaacaagaggacccgtactgcctacacccgggcgcagctgctggagctggagaaggaattcttatttaacaaatacatctcccggccccgccgggtggagctggcagtgatgttgaacttgaccgagagacacatcaaaatctggttccaaaaccgtcgcatgaagtggaaaaaagaggaagataagaaacgtagtagcgggaccccgagtgggggcggtgggggcgaagagccggagcaagattgtgcggtgacctcgggcgaggagctgctggcagtgccaccgctgccacctcccggaggtgccgtgcccccaggcgtcccagctgcagtccgggagggcctactgccttcgggccttagcgtgtcgccacagccctccagcatcgcgccactgcgaccgcaggaaccccggtgaggacagcagtctgagggtgagcgggtctgggacccagagtgtggacgtgggagcgggcagctggataagggaacttaacctaggcgtcgcacaagaagaaaattcttgagggcacgagagccagttgggtatagccggagagatgctggcagacttctggaaaaacagccctgagcttctgaaaactttgaggctgcttctgatgccaagcgaatggccagatctgcctctaggactctttcctgggaccaatttagacaacctgggctccaaactgaggacaataaaaagggtacaaacttgagcgttccaatacggaccagcaggcgag